(19) United States
(12) Patent Application Publication
(10) Pub. No.: US 2026/0348950 A1
(43) Pub. Date: Nov. 5, 2026

(54) FENTANYL-SPECIFIC SINGLE VARIABLE-DOMAIN ANTIBODIES AND USE IN A CONTINUOUS AGGLUTINATION ASSAY

(71) Applicant: Quantitative BioSciences, Inc., San Diego, CA (US)

(72) Inventors: Nicholas Sigmund Csicsery, San Diego, CA (US); Michael Stephen Ferry, San Diego, CA (US); Scott Warren Cookson, San Diego, CA (US); Natalie Anne Cookson, San Diego, CA (US); William H. Mather, San Diego, CA (US)

(73) Assignee: Quantitative BioSciences, Inc., San Diego, CA (US)

(21) Appl. No.: 19/109,124

(22) PCT Filed: Sep. 3, 2023
(86) PCT No.: PCT/US2023/031942

§ 371 Date: Mar. 5, 2025

Related U.S. Application Data
(60) Provisional application No. 63/404,495, filed on Sep. 7, 2022.

(51) Int. Cl. G01N 33/53; G01N 21/82; G01N 33/94

(52) U.S. Cl. CPC G01N 33/5308; G01N 21/82; G01N 33/5304; G01N 33/9486; G01N 2021/825

(57) ABSTRACT

Disclosed are isolated recombinant antigen-binding proteins (including embodiments comprising a V_{HH} domain) that specifically binds fentanyl or carfentanil. Also disclosed are isolated nucleic acids that encode the antigen-binding proteins and expression vectors and host cells, in culture, that comprise them, and a method of producing the antigen-binding protein. Also disclosed are devices for detecting the presence of fentanyl and/or carfentanil, pharmaceutical compositions, formulations, and a method of treating a patient addicted to opiates, that employ the antigen-binding proteins. A continuous agglutination assay method for detecting an analyte of interest, such as fentanyl and/or carfentanil, is also disclosed, as well as an automated system for continuous agglutination assay.

### STATEMENT OF GOVERNMENT LICENSE RIGHTS

This invention was made with government support under Contract No. CWMD1916-003 awarded by the United States Department of Defense. The government has certain rights in the invention.

### SEQUENCE LISTING

The instant application contains a Sequence Listing which has been submitted electronically in XML file format and is hereby incorporated by reference in its entirety. Said XML copy, created on Jul. 6, 2023, is named QBI02PCT_SL.xml and is 59,797 bytes in size.

### BACKGROUND OF THE INVENTION

### 1. Field of the Invention

This invention relates to antigen-binding proteins that specifically bind opioids and opioid derivatives, such as fentanyl and carfentanil.

### 2. Discussion of the Related Art

Opioids are substances that act on opioid receptors to produce morphine-like analgesic effects in humans and other mammals. Opioids are most often used medically to relieve pain, and by people addicted to opioids. Opioids include opiates, an older term that refers to such drugs derived from opium, including morphine itself. Other opioids are semi-synthetic and synthetic drugs such as hydrocodone (aka dihydrocodeinone, 4,5α-epoxy-3-methoxy-17-methylmorphinan-6-one); oxycodone (aka dihydrohydroxycodeinone, 6-deoxy-7,8-dihydro-14-hydroxy-3-O-methyl-6-oxomorphine); fentanyl (CAS #437-38-7; also known as fentanyl, or N-(1-(2-phenethyl)-4-piperidinyl-N-phenyl-propanamide, or 1-(2-Phenylethyl)-4-(N-propanilido)piperidine); carfentanil (CAS #59708-52-0; Methyl 1-(2-phenylethyl)-4-[phenyl(propanoyl)amino]piperidine-4-carboxyl-ate); acetyl fentanyl (N-(1-Phenethylpiperidin-4-yl)-N-phenylacetamide); and synthetic opioids that are utilized in treating opioid addiction, such as methadone ((RS)-6-(dimethylamino)-4,4-diphenylheptan-3-one) and buprenorphine ((2S)-2-[(5R,6R,7R,14S)-17-cyclopropylmethyl-4,5-epoxy-6,14-ethano-3-hydr-oxy-6-methoxymorphinan-7-yl]-3,3-dimethylbutan-2-ol); and antagonist drugs such as naloxone and endogenous peptides, such as the endorphins.

Opioids produce their pharmacological effects through activation of G protein-coupled receptors (GPCRs). There are four distinct genes coding for opioid receptors: the mu-, kappa-, and delta-opioid receptors (MOR, KOR, and DOR, respectively) and the opioid-like receptor1 (ORL-1) or the nociceptine receptor (NOP). The generation of genetic knockout mice has demonstrated that the majority of clinically used opioids including morphine produce their pharmacological effects primarily by activating the MOR. The MOR is widely distributed and expressed in neurons in the brain, spinal cord, and the periphery (Gutstein and Akil 2001). While effective in reducing patient pain and discomfort, many of the opioid substances, acting upon the opioid receptors are also highly addictive, and may be lethal at relatively low doses. For example, two milligrams of fentanyl can be lethal depending on a person's body size, tolerance and past usage.

According to the United States Drug Enforcement Agency (DEA), illicit fentanyl, primarily manufactured in foreign clandestine labs and smuggled into the United States through Mexico, is being distributed across the United States and is being sold on the illegal drug market. Fentanyl is being mixed in with other illicit drugs to increase the potency of the drug, sold as powders and nasal sprays, and increasingly pressed into pills made to look like legitimate prescription opioids. Because there is no official oversight or quality control, these counterfeit pills often contain lethal doses of fentanyl, with none of the promised drug. There is significant risk that illegal drugs have been intentionally contaminated with fentanyl. Because of its potency and low cost, drug dealers have been mixing fentanyl with other drugs including heroin, methamphetamine, and cocaine, increasing the likelihood of a fatal interaction. Analysis by the DEA of seized material has found counterfeit pills ranging from 0.02 to 5.1 milligrams (more than twice the lethal dose) of fentanyl per tablet. According to the Center for Disease Control (CDC), synthetic opioids (like fentanyl) are the primary driver of the growing number of overdose deaths in the United States.

Drug trafficking organizations typically distribute fentanyl by the kilogram. One kilogram of fentanyl has the potential to kill 500,000 people, and there is a risk that residual amounts of fentanyl can find its way to environmental surfaces and waters, with potentially harmful effects.

A comparative study of old world camelids (Camelus bactrianus and Camelus dromedarius) and new world camelids (Lama pacos, Lama glama and Lama vicugna) showed that heavy chain immunoglobulins are abundant in the sera of all camelid species examined and total up to 75% of the molecules binding to Protein A; these immunoglobulin molecules are composed of heavy chain dimers, comprising V_{HH} domains, and are devoid of light chains. (Casterman et al., Immunoglobulins devoid of light chains, U.S. Pat. No. 5,874,541; Hamers et al., Production of antibodies or (functionalized)fragments thereof derived from heavy chain immunoglobulins of Camelidae, US2005/0130266A1, U.S. Pat. No. 7,794,981B2).

There are many advantages to nanobodies as ABPs, mainly relating to their small size and stability. Nanobodies are roughly 1/10^{th} the size of conventional antibodies, with a molecular weight of about 15 kDa, while retaining comparable antigen binding affinities. The small size of nanobodies enables cheaper production, easier bacterial expression and surface display, and expanded applications where ABP size is relevant to delivery (e.g., drug delivery). Nanobodies are also remarkably stable to environmental conditions, in some cases with melting temperatures observed above 80 °C and refolding after denaturation. (Goldman et al., “Enhancing Stability of Camelid and Shark Single Domain Antibodies: An Overview,” Front Immunol. 2017 Jul. 25; 8:865. doi: 10.3389/fimmu.2017.00865. eCollection 2017).

Thus, the present invention provides, inter alia, robust assay tools for the detection of fentanyl, carfentanil, and other analytes, including with effective continuous sample monitoring systems and methods of the invention.

### SUMMARY OF THE INVENTION

In one aspect the present invention relates to an isolated recombinant antigen-binding protein that specifically binds fentanyl or carfentanil, comprising a V_{HH} domain comprising a set of three complementarity determining regions (CDR): CDR1, CDR2, and CDR3, wherein each CDR comprises an amino acid sequence, wherein the set of three CDRs is selected from the group consisting of:
(a) SEQ ID NO: 2, SEQ ID NO: 3, and SEQ ID NO: 4;
(b) SEQ ID NO: 2, SEQ ID NO: 3, and SEQ ID NO: 44;
(c) SEQ ID NO: 6, SEQ ID NO: 7, and SEQ ID NO: 8;
(d) SEQ ID NO: 12, SEQ ID NO: 13, and SEQ ID NO: 14;
(e) SEQ ID NO: 17, SEQ ID NO: 18, and SEQ ID NO: 19;
(f) SEQ ID NO: 25, SEQ ID NO: 26, and SEQ ID NO: 27;
(g) SEQ ID NO: 25, SEQ ID NO: 26, and SEQ ID NO: 51;
(h) SEQ ID NO: 30, SEQ ID NO: 31, and SEQ ID NO: 32;
(i) SEQ ID NO: 34, SEQ ID NO: 35, and SEQ ID NO: 36;
(j) SEQ ID NO: 40, SEQ ID NO: 41, and SEQ ID NO: 42; and
(k) SEQ ID NO: 55, SEQ ID NO: 56, and SEQ ID NO: 57;
or wherein one or more of the CDR amino acid sequences of the set of three CDR amino acid sequences comprises a conservative amino acid substitution of one or two, preferably one, amino acid residues; and wherein the antigen binding protein does not specifically bind nor fentanyl; and, optionally, wherein the isolated recombinant antigen-binding protein specifically binds fentanyl or carfentanil with an equilibrium dissociation constant (K_{d} or K_{D}) of ≤10⁻⁸ M, more preferably with a K_{d} or K_{D} of ≤5×10⁻⁹ M, and even more preferably with a K_{d} or K_{D} of ≤5×10⁻¹⁰ M, or even ≤10⁻¹⁰ M, as measured, e.g., by enzyme-linked immunosorbent assay (hereinafter “ELISA”) or surface plasmon resonance.

In another aspect the present invention relates to an isolated nucleic acid, comprising a nucleotide sequence encoding the inventive recombinant antigen-binding protein, to an expression vector comprising the nucleic acid, and to a host cell, in culture, comprising the expression vector. Such host cells can be used in a method of producing an antigen-binding protein that specifically binds to fentanyl and/or carfentanil. These V_{HH} domain antibodies (or “nanobodies”) can be used as diagnostic tools, or in a pharmaceutical composition as a therapeutic in the treatment of a patient addicted to opiates (e.g., fentanyl) or the event of opiate overdose.

In still another aspect, the present invention is directed to the inventive recombinant antigen-binding proteins being expressed on the surface of a host cell, e.g., a microorganism. For example, in some embodiments the inventive antigen-binding proteins can be used for real-time detection of fentanyl and/or carfentanil, employing synthetic biology to integrate fentanyl-binding nanobodies into gene circuits in microbial cells, such as E. coli, to develop biosensor strains that emit a signal in the presence of fentanyl. These can be employed in immunological assays, such as lateral flow or agglutination assays.

Accordingly, the present invention also relates to a continuous agglutination assay method for detecting an analyte of interest, which can be fentanyl and/or carfentanil, or a different analyte of interest. The continuous agglutination assay method includes the following steps:
(a) mixing in a reaction vessel, a fluid aqueous suspension of host cells that display on their surfaces a plurality of recombinant antigen-binding proteins that specifically bind an analyte of interest, with a fluid aqueous sample, in a reaction mixture with an agglutinating agent comprising the analyte of interest, or an analyte conjugate, under conditions of temperature and pH that permit binding of the analyte or the analyte conjugate, by the antigen-binding protein and agglutination of the cells, in the presence of the analyte of interest;
(b) measuring a parameter in a preselected portion of the reaction mixture over a continuous time course;
(c) correlating the change in the measured parameter over the time course with the level of agglutination; and
(d) normalizing the level of agglutination relative to maximum and minimum control levels of agglutination to obtain a normalized value; wherein the normalized value reflects the concentration of the analyte of interest in the aqueous sample over the continuous time course. The continuous agglutination assay method can be practiced using an automated system for continuous agglutination assay, which the present invention also provides. The inventive automated system for continuous agglutination assay includes the following:
(a) a first vessel configured to receive and to contain, a fluid aqueous suspension comprising host cells that display on their surfaces a plurality of recombinant antigen-binding proteins that specifically bind an analyte of interest, and, wherein the fluid aqueous suspension optionally comprises an antibiotic or bacteriostatic compound, a coagulant and/or an agglutination-enhancing additive;
(b) an optional second vessel configured to receive and to contain a fluid aqueous sample to be analyzed;
(c) a reaction vessel fluidly connected to the first vessel and to the optional second vessel by connecting lines, wherein the reaction vessel is configured to automatically receive via the connecting lines, a predetermined volume of the fluid aqueous suspension from the first vessel and a predetermined volume of the fluid aqueous sample, optionally from the second vessel, or directly from a sample inlet, and to contain a reaction mixture comprising the host cells and the aqueous sample;
(d) a sensor configured to measure a parameter correlatable to the level of agglutination in the reaction mixture in the reaction vessel, in response to a measurement signal, and to transmit the thusly acquired measurement data;
(e) a waste outlet, or optionally a waste vessel, fluidly connected to the reaction vessel by a connecting line;
(f) a plurality of fluid propellers each configured to impel fluid flow through the connecting lines in response to a signal, wherein at least one fluid propeller is upstream from the reaction vessel, and at least one fluid propeller is downstream from the reaction vessel;
(g) a microcontroller configured and programmable to signal the plurality of fluid propellers to initiate fluid flow, impel fluid flow, or cease fluid flow, through the connecting lines:
(i) from the first vessel, and from the optional second vessel or the sample inlet, to the reaction vessel;
(ii) from the reaction vessel to the waste outlet, or optionally to the waste vessel; and
   wherein the microcontroller, or optionally, a separate microcontroller, is further configured and programmable to transmit the measurement signal to the sensor, and to collect the measurement data from the sensor. Thus, the measurement data pertaining to the parameter correlated to the agglutination level in the reaction vessel allow the continuous monitoring and detection of an analyte of interest, such as, but not limited to, fentanyl and/or carfentanil.

The foregoing summary is not intended to define every aspect of the invention, and additional aspects are described in other sections, such as the Detailed Description of Embodiments. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows results of the initial ELISA analysis of V_{HH} sequences isolated from library panning, as described in Example 1 herein. The left panel (Panel (a)) shows ELISA results for 94 recovered nanobody proteins, targeted by an anti-alpaca V_{HH} domain antibody. The net difference in absorbance (of 405 nm wavelength light) between a plate coated with fentanyl-BSA conjugate and unconjugated BSA is shown. The negative control well contains no nanobody and the positive control well was coated with a nanobody. Error bars represent standard deviation of V_{HH} sequences present in two or more wells. The right panel (Panel (b)) shows the number of replicate wells containing each of the 28 unique V_{HH} sequences from the 94 total colonies screened.

FIG. 2 represents the results of multiple sequence alignments and agglomerative clustering of the unique V_{HH} sequences using Clustal Omega software. Four families were found among the 28 unique sequences, among which each V_{HH} shared at least 2 CDR sequences. Family 1: (SEQ ID NO: 2), (SEQ ID NO: 3), and (SEQ ID NO: 4), designated in FIG. 2 as “SEQ 2,” “SEQ 3,” and “SEQ 4,” respectively. Family 2: (SEQ ID NO: 12), (SEQ ID NO: 13), and (SEQ ID NO: 14), designated in FIG. 2 as “SEQ 12,” “SEQ 13,” and “SEQ 14,” respectively. Family 3: (SEQ ID NO: 6), (SEQ ID NO: 7), and (SEQ ID NO: 8), designated in FIG. 2 as “SEQ 6,” “SEQ 7,” and “SEQ 8,” respectively. Family 4: (SEQ ID NO: 25), (SEQ ID NO: 26), and (SEQ ID NO: 27), designated in FIG. 2 as “SEQ 25,” “SEQ 26,” and “SEQ 27,” respectively.

FIG. 3 shows normalized absorbance (of 600 nm wavelength light) of cell cultures after a 30-minute agglutination reaction, incubating cell cultures with 0.5 nM of fentanyl-BSA (“0.5 nM fen.-BSA”; white bars) and 200 ppb of either unbound fentanyl (“200 ppb fen.”; diagonal hatched bars) or unbound carfentanil (“200 ppb carfen.”; cross-hatched bars), as described in Example 2 herein. Absorbance for each strain is normalized to the no fentanyl-BSA condition (“No fen.-BSA”; gray bars). Error bars represent the standard deviation of triplicate tests.

FIG. 4 shows results from agglutination of the A06 sdAb-expressing E. coli strain at varying opioid concentrations, as described in Example 2 herein. Relative agglutination is defined such that the maximum absorbance (of 600-nm wavelength of light) measured at 30 minutes is equal to 1, and the lowest absorbance is equal to 0. Cross-reactivity relative to fentanyl and the concentration of inhibitor necessary to achieve 50% agglutination (IC₅₀) is displayed for fentanyl (left panel), carfentanil (middle panel), or nor fentanyl (right panel).

FIG. 5 shows results from whole-cell ELISA assays for the estimation of binding affinity of E. coli surface-displayed sdAbs, as described in Example 3 herein.

FIG. 6 shows the results of ELISA of proteins in cell-free extracts for the estimation of binding affinity, as described in Example 4 herein.

FIG. 7a-d schematically illustrates an exemplary embodiment of the inventive continuous agglutination assay method for detecting an analyte of interest, as further described in Example 5 herein. FIG. 7(a), left panel shows a schematic close-up representation of the agglutination reaction vessel (i.e., designated the “Cuvette” in FIG. 7b). FIG. 7a, right upper panel, illustrates the expected behavior of agglutinating and non-agglutinating cell suspensions in an embodiment employing transmitted light as the turbidity sensing modality, with turbidity decreasing from the beginning of the reaction (FIG. 7a, right lower panel, “t=0”) through to the end of the reaction (FIG. 7a, right lower panel, “t=end”), as agglutinated immunoaggregates settle to the bottom of the reaction vessel (“Cuvette”). FIG. 7b shows schematically an exemplary embodiment of the parts and instrumentation for the hardware involved in the inventive continuous agglutination assay method or system (or device), including the reaction vessel (“Cuvette”), pumps, and optional aqueous sample reservoir (shown schematically as “Water source”) and a reagent reservoir (shown here schematically as a single “Cell suspension” reservoir), although separate vessels or reservoirs for other diluents, buffers, and/or reagents can optionally be included in the assay system, with the entire assay system being under the control of the “Microcontroller,” which automatically directs the activity of the pumps, turbidity sensor apparatus, and data collection. FIG. 7b also shows a “turbidity sensor” (e.g., a laser and photodiodes), but other parameters, correlatable with the level of agglutination, can be selected for measurement instead of turbidity, e.g., transmission, scattering, or absorbance of light, or other parameters measurable with a piezoelectric detector or a surface plasmon resonance sensor. FIG. 7c shows a representative sample time series of the continuous agglutination assay method employing anti-fentanyl sdAb-displaying E. coli cells with 3 nM fentanyl-BSA conjugate, as increasing concentrations of unbound fentanyl were added to the “Cuvette” over time. FIG. 7d shows a representative response curve for the continuous competitive fentanyl assay extracted from the data in FIG. 7c. Values were extracted after 30 minutes of the agglutination reaction, with an agglutination value representing the mean change in transmitted light of reactions containing 0 ppb fentanyl, and an agglutination value of 0 representing no change in transmitted light over the 30 minutes. This exemplified the utility of an analyte conjugate (fentanyl-BSA) to induce agglutination, while the unconjugated fentanyl disrupted agglutination. The inventive competitive assay can be performed with a variety of conjugates of the analyte of interest, e.g., protein or polypeptide conjugates (such as but not limited to BSA, OVA, KLH, 6His), or PEG conjugates. A typical detectable concentration range for the analyte, or analyte conjugate, is about 10-100 ppb, and suitable dilution of the analyte, or analyte conjugate, with water or buffer, can be used to bring the sample concentration within maximum range of sensitivity of the instrumentation/detector(s) that are employed.

FIG. 8a-b shows representative data obtained from the inventive continuous agglutination assay method, in which the analyte of interest was a protein target (e.g., an antibody), as further described in Example 6 herein. FIG. 8a shows an agglutination time series at varying concentrations of purified mouse IgG1, κ isotype control antibody (BioLegend, Cat. No. 400101). Each agglutination reaction was allowed to proceed for 45 minutes. FIG. 8b shows bar plots of the mean agglutination at increasing antibody concentrations. Error bars represent the standard deviation of triplicate samples.

FIG. 9 shows a schematic illustration of embodiment of a continuous, real-time system was also developed with pressurized gas directed into the headspace of the respective cell suspension reservoir(s) (“Vessel 1”) and the optional aqueous sample reservoir (“Vessel 2”), to control the flow of agglutinating agent and fluid aqueous sample into the reaction vessel. The arrows point in the direction of gas and fluid flow. Two electro-pneumatic gas pressure regulators (0.15-15 psi) for the gas plumbing system are shown.

### DETAILED DESCRIPTION OF EMBODIMENTS

The section headings used herein are for organizational purposes only and are not to be construed as limiting the subject matter described.

### DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a,” “an,” and “the,” include plural referents unless the context clearly indicates otherwise. For example, reference to “a protein” includes a plurality of proteins; reference to “a cell” includes populations of a plurality of cells.

In some embodiments of the present invention, the isolated recombinant antigen-binding protein that specifically binds fentanyl or carfentanil, is a single variable-domain antibody (“sdAb” or “nanobody” or “Nb,” terms used interchangeably herein), devoid of a light chain, specifically binding to fentanyl and/or carfentanil. In a particular embodiment, the single domain antibody is derived from camelids. In the family of “camelids,” immunoglobulins devoid of light polypeptide chains are found. “Camelids” comprise old-world camelids (Camelus bactrianus and Camelus dromaderius) and new world camelids (for example, Lama paccos, Lama glama and Lama vicugna).

The term “immunoglobulin” encompasses a gamut of antibody molecules, including full antibodies comprising two dimerized heavy chains (HC), each covalently linked to a light chain (LC); a single undimerized immunoglobulin heavy chain and covalently linked light chain (HC+LC; i.e., monomeric Ab), or a chimeric immunoglobulin (light chain+heavy chain)-Fc heterotrimer (a so-called “hemibody”), or a single domain antibody or nanobody. An “immunoglobulin” is a protein, but is not necessarily an antigen-binding protein, for it may also be engineered not to have a known target or may naturally not specifically bind to a known target.

It should be noted that the term “nanobody,” as used herein in its broadest sense, is not limited to a specific biological source or to a specific method of preparation. For example, the antigen binding proteins of the present invention can generally be obtained: (1) by isolating the V_{HH} domain of a naturally occurring heavy chain antibody; (2) by expression of a nucleotide sequence encoding a naturally occurring V_{HH} domain; (3) by “humanization” of a naturally occurring V_{HH} domain or by expression of a nucleic acid encoding a such humanized V_{HH} domain; (4) by “camelization” of a naturally occurring VH domain from any animal species and, in particular, from a mammalian species, such as from a human being, or by expression of a nucleic acid encoding such a camelized VH domain; (5) by “camelization” of a “domain antibody” or “Dab” as described in the art, or by expression of a nucleic acid encoding such a camelized V_{H} domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing. One preferred class of nanobodies corresponds to the V_{HH} domains of naturally occurring heavy chain antibodies directed against fentanyl and/or carfentanil. As further described herein, such V_{HH} sequences can generally be generated or obtained by suitably immunizing a species of camelid with fentanyl or carfentanil (i.e., so as to raise an immune response and/or heavy chain antibodies directed against fentanyl and/or fentanyl), by obtaining a suitable biological sample from the camelid (such as a blood sample, serum sample or sample of B-cells), and by generating V_{HH} sequences directed against fentanyl (or carfentanil), starting from the sample, using any suitable technique known per se. Such techniques will be clear to the skilled person.

An “isolated” protein, e.g., an antibody protein, is one that has been identified and separated from one or more components of its natural environment or of a culture medium in which it has been secreted by a producing cell. In some embodiments, the isolated protein is substantially free from proteins or polypeptides or other contaminants that are found in its natural or culture medium environment that would interfere with its therapeutic, diagnostic, prophylactic, research or other use. “Contaminant” components of its natural environment or medium are materials that would interfere with diagnostic or therapeutic uses for the protein, e.g., an antibody, and may include enzymes, hormones, and other proteinaceous or nonproteinaceous (e.g., polynucleotides, lipids, carbohydrates) solutes. Typically, an “isolated protein” constitutes at least about 5%, at least about 10%, at least about 25%, or at least about 50% of a given sample. In some embodiments, the protein of interest, e.g., an antibody, will be purified (1) to greater than 95% by weight of protein, and most preferably more than 99% by weight, or (2) to homogeneity by SDS-PAGE, or other suitable technique, under reducing or nonreducing conditions, optionally using a stain, e.g., Coomassie blue or silver stain. Isolated naturally occurring antibody includes the antibody in situ within recombinant cells since at least one component of the protein's natural environment will not be present. Typically, however, the isolated protein of interest (e.g., an antibody) will be prepared by at least one purification step. The term “naturally occurring,” where it occurs in the specification in connection with biological materials such as polypeptides, nucleic acids, host cells, and the like, refers to materials which are found in nature.

A “domain” or “region” (used interchangeably herein) of a polynucleotide is any portion of the entire polynucleotide, up to and including the complete polynucleotide, but typically comprising less than the complete polynucleotide. A domain can, but need not, fold independently (e.g., DNA hairpin folding) of the rest of the polynucleotide chain and/or be correlated with a particular biological, biochemical, or structural function or location, such as a coding region or a regulatory region.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

“Antigen binding region” or “antigen binding site” means a portion of a protein that specifically binds a specified target ligand or antigen, e.g., fentanyl and/or carfentanil. For example, that portion of an antigen binding protein that contains the amino acid residues that interact with a target ligand or an antigen and confer on the antigen binding protein its specificity and affinity for the antigen is referred to as “antigen binding region.” In an antibody, an antigen binding region typically includes one or more “complementarity determining regions” (“CDRs”). Certain antigen binding regions also include one or more “framework” regions (“FRs”). A “CDR” is an amino acid sequence that contributes to antigen binding specificity and affinity. “Framework” regions can aid in maintaining the proper conformation of the CDRs to promote binding between the antigen binding region and an antigen. In a traditional antibody, the CDRs are embedded within a framework in the heavy and light chain variable region where they constitute the regions responsible for antigen binding and recognition. A variable region of an immunoglobulin antigen binding protein comprises at least three heavy or light chain CDRs, see, supra (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991, supra; see also Chothia and Lesk, 1987, supra).

The term “target” or “antigen” refers to a molecule or a portion of a molecule capable of being bound by a selective binding agent, such as an antigen binding protein (including, e.g., an antibody or immunologically functional fragment of an antibody), and additionally capable of being used in an animal to produce antibodies capable of binding to that antigen. An antigen may possess one or more epitopes that are capable of interacting with different antigen binding proteins, e.g., antibodies.

The term “epitope” is the portion of a target molecule that is bound by an antigen binding protein (for example, an antibody or antibody fragment). The term includes any determinant capable of specifically binding to an antigen binding protein, such as an antibody or to a T-cell receptor. An epitope can be contiguous or non-contiguous (e.g., in a single-chain polypeptide, amino acid residues that are not contiguous to one another in the polypeptide sequence but that within the context of the molecule are bound by the antigen binding protein). In certain embodiments, epitopes may be mimetic in that they comprise a three-dimensional structure that is similar to an epitope used to generate the antigen binding protein, yet comprise none or only some of the amino acid residues found in that epitope used to generate the antigen binding protein. Most often, epitopes reside on proteins, but in some instances they may reside on other kinds of molecules, such as nucleic acids. Epitope determinants may include chemically active surface groupings of molecules such as amino acids, sugar side chains, phosphoryl or sulfonyl groups, and they may have specific three-dimensional structural characteristics, and/or specific charge characteristics. Generally, antigen binding proteins specific for a particular target will preferentially recognize an epitope on the target in a complex mixture of proteins and/or macromolecules.

An “analyte conjugate” is a molecule of the analyte of interest, or target or epitope portion of the analyte molecule of interest, that is covalently bound to another moiety selected for added convenience in purification or detection operations, a moiety such as but not limited to, bovine serum albumin (BSA), ovalbumin (OVA), keyhole limpet hemocyanin (KLH), polyethylene glycol (PEG), hexa-histidine (6His or 6-His) other versions of a poly-histidine tag, or the like. For example, if the analyte of interest is fentanyl or carfentanil, an exemplary “analyte conjugate” can be fentanyl or carfentanil covalently bound to BSA, OVA, KLH, 6His, or PEG.

A protein of interest, such as an antigen-binding protein (e.g., sdAb, nanobody, or other antibody or antibody fragment) used in the practice of the invention, whether a variant or parent protein (e.g., an antibody, sdAb, or nanobody), is typically produced by recombinant expression technology. The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. Examples of such molecular biological procedures are found in Maniatis et al., Molecular Cloning. A Laboratory Manual. Cold Spring Harbor Laboratory, Cold Spring Harbor, N.Y. (1982). A “recombinant DNA molecule,” is comprised of segments of DNA joined together by means of such molecular biological techniques.

The term “recombinant protein” or “recombinant polypeptide” as used herein refers to a protein molecule, e.g., an antibody, which is expressed using a recombinant DNA molecule. A “recombinant host cell” is a cell that contains and/or expresses a recombinant nucleic acid.

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

A “promoter” is a region of DNA including a site at which RNA polymerase binds to initiate transcription of messenger RNA by one or more downstream structural genes. Promoters are located near the transcription start sites of genes, on the same strand and upstream on the DNA (towards the 5′ region of the sense strand). Promoters are typically about 100-1000 bp in length.

An “enhancer” is a short (50-1500 bp) region of DNA that can be bound with one or more activator proteins (transcription factors) to activate transcription of a gene.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

A protein of interest for purposes of the present invention, whether it includes a variant or parental amino acid sequence, is typically produced by recombinant expression technology, although it can also be a naturally occurring protein.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be expressed recombinantly using known protein engineering techniques. In addition, proteins can be derivatized as described herein and by other well-known organic chemistry techniques.

The term “purify” or “purifying” a protein means increasing the degree of purity of the desired protein from a composition or solution comprising the protein of interest (i.e., the “POI,” and one or more contaminants by removing (completely or partially) at least one contaminant from the composition or solution. An “isolated” protein is one that has been identified and separated from one or more components of its natural environment or of a culture medium in which it has been secreted by a producing cell. In some embodiments, the isolated protein is substantially free from proteins or polypeptides or other contaminants that are found in its natural or culture medium environment that would interfere with its therapeutic, diagnostic, prophylactic, research or other use. “Contaminant” components of its natural environment or medium are materials that would interfere with industrial, research, therapeutic, prophylactic, or diagnostic or uses for the protein of interest, and may include enzymes, hormones, and other proteinaceous or nonproteinaceous (e.g., polynucleotides, lipids, carbohydrates) solutes. Typically, an “isolated protein” or, interchangeably, “protein isolate,” constitutes at least about 5%, at least about 10%, at least about 25%, or at least about 50% of a given sample. In some embodiments, the isolated protein of interest will be “purified”: (1) to greater than 95% by weight of protein, and most preferably, more than 99% by weight, or (2) to homogeneity by SDS-PAGE, or other suitable technique, under reducing or nonreducing conditions, optionally using a stain, e.g., Coomassie blue or silver stain. An isolated naturally occurring antibody includes the antibody in situ within recombinant cells since at least one component of the protein's natural environment will not be present. Typically, however, the isolated or purified protein of interest (e.g., a purified protein drug substance) will be prepared by at least one purification step, which can include cell lysis (with or without centrifugation), filtration, and/or affinity chromatography.

A “variant” of a polypeptide (e.g., an immunoglobulin, or an antibody) comprises an amino acid sequence wherein one or more amino acid residues are inserted into, deleted from and/or substituted into the amino acid sequence relative to another polypeptide sequence. Variants can include fusion proteins.

The term peptide or protein “analog” refers to a polypeptide having a sequence that differs from a peptide sequence existing in nature by at least one amino acid residue substitution, internal addition, or internal deletion of at least one amino acid, and/or amino- or carboxy-terminal end truncations, or additions). An “internal deletion” refers to absence of an amino acid from a sequence existing in nature at a position other than the N- or C-terminus. Likewise, an “internal addition” refers to presence of an amino acid in a sequence existing in nature at a position other than the N- or C-terminus.

The term “fusion protein” indicates that the protein includes polypeptide components derived from more than one parental protein or polypeptide. Typically, a fusion protein is expressed from a “fusion gene” in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein. Fusion proteins incorporating an antibody or an antigen-binding portion thereof are known.

A “secreted” protein refers to those proteins capable of being directed to the endoplasmic reticulum (ER), secretory vesicles, or the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage. In some other embodiments, the antibody protein of interest can be synthesized by the host cell as a secreted protein, which can then be further purified from the extracellular space and/or medium.

As used herein “soluble” when in reference to a protein produced by recombinant DNA technology in a host cell is a protein that exists in aqueous solution; if the protein contains a twin-arginine signal amino acid sequence the soluble protein is exported to the periplasmic space in gram negative bacterial hosts, or is secreted into the culture medium by eukaryotic host cells capable of secretion, or by bacterial host possessing the appropriate genes (e.g., the kil gene). Thus, a soluble protein is a protein which is not found in an inclusion body inside the host cell. Alternatively, depending on the context, a soluble protein is a protein which is not found integrated in cellular membranes, or, in vitro, is dissolved, or is capable of being dissolved in an aqueous buffer under physiological conditions without forming significant amounts of insoluble aggregates (i.e., forms aggregates less than 10%, and typically less than about 5%, of total protein) when it is suspended without other proteins in an aqueous buffer of interest under physiological conditions, such buffer not containing an ionic detergent or chaotropic agent, such as sodium dodecyl sulfate (SDS), urea, guanidinium hydrochloride, or lithium perchlorate. In contrast, an insoluble protein is one which exists in denatured form inside cytoplasmic granules (called an inclusion body) in the host cell, or again depending on the context, an insoluble protein is one which is present in cell membranes, including but not limited to, cytoplasmic membranes, mitochondrial membranes, chloroplast membranes, endoplasmic reticulum membranes, etc., or in an in vitro aqueous buffer under physiological conditions forms significant amounts of insoluble aggregates (i.e., forms aggregates equal to or more than about 10% of total protein) when it is suspended without other proteins (at physiologically compatible temperature) in an aqueous buffer of interest under physiological conditions, such buffer not containing an ionic detergent or chaotropic agent, such as sodium dodecyl sulfate (SDS), urea, guanidinium hydrochloride, or lithium perchlorate.

The term “polynucleotide” or “nucleic acid,” used interchangeably herein, includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoranilidate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express the immunoglobulin (e.g., antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

An expression cassette is a typical feature of recombinant expression technology. The expression cassette includes a gene encoding a protein of interest, e.g., a gene encoding an antibody sequence, such as an immunoglobulin light chain and/or heavy chain sequence. A eukaryotic “expression cassette” refers to the part of an expression vector that enables production of protein in a eukaryotic cell, such as a mammalian cell. It includes a promoter, operable in a eukaryotic cell, for mRNA transcription, one or more gene(s) encoding protein(s) of interest and a mRNA termination and processing signal. An expression cassette can usefully include among the coding sequences, a gene useful as a selective marker or reporter. In the expression cassette promoter is operably linked 5′ to an open reading frame encoding an exogenous protein of interest; and a polyadenylation site is operably linked 3′ to the open reading frame. Other suitable control sequences can also be included as long as the expression cassette remains operable. The open reading frame can optionally include a coding sequence for more than one protein of interest.

A “reporter protein” as described herein, refers to a protein that is detected which is indicative of transcription or translation from a regulatory sequence of interest in a bacteria, cell culture or animal. A reporter gene is a gene that is attached to a regulatory sequence of another gene. These can be used to indicate whether a certain gene is expressed in the presence of an analyte. Without being limiting common reporter genes to express a reporter proteins can be green fluorescent protein, luciferase (which can catalyze a reaction with luciferin to produce light, and red fluorescent protein. Without being limiting a common reporter in bacteria is E. coli lacZ gene, which encodes beta-galactosidase which can cause bacteria to appear in a blue color when grown in a medium that contains the substrate X-gal.

In regards to an “electrochemical reaction product” detection method, in some embodiments there has been success in detecting hydrogen peroxide (H₂O₂) produced by reactive oxygen species formed when green fluorescent protein molecules are illuminated within their excitation spectrum. This H₂O₂ is detected at microelectrodes integrated into the microfluidic device. The microelectrodes may be functionalized by coating them with a thin film (for example, Prussian blue) to increase sensitivity and selectivity. They may also be coated with a protectant (for example, Nafion) to prevent fouling. Microelectrodes may be positioned in the same fluidic channel as the cells or in an adjacent fluidic channel, separated by a thin barrier of PDMS. The latter sensing methodology may limit chemical fouling of the microelectrode surface over long measurement durations and is feasible due to the ability of H₂O₂ to diffuse through PDMS. In some embodiments, an electrochemical reaction product is product that can produce a detectable electric current. These types of reactions can involve electric charges that can move between the electrode and the electrolyte. In some embodiments, of the microfluidic devices described herein, the microfluidic devices comprise microelectrodes integrated into the microfluidic device. In some embodiments, the microelectrodes may be functionalized by coating them with a thin film (e.g. Prussian blue) to increase sensitivity and selectivity. In some embodiments, the microelectrodes are coated with a protectant (e.g. Nafion) to prevent fouling. In some embodiments, the microelectrodes are positioned in the same fluidic channel as the cells or in an adjacent fluidic channel, separated by a thin barrier of PDMS.

“Enzymatic assay product” as described herein, can be a product or a protein that is usually detected from an enzymatic reaction. Without being limiting, one example would be to engineer the cells to produce the beta-galactosidase enzyme (e.g. lacZ for bacteria). The medium can then be supplemented with the organic compound X-gal (BCIG, for 5-bromo-4-chloro-3-indolyl-β-D-galactopyranoside), and the beta-galactosidase enzyme would hydrolyze this to an insoluble blue compound that is detectable by an imaging system. Alternatively, another way to assay for the enzymatic assay product is to engineer the cells to produce the beta-galactosidase enzyme. The medium would then be supplemented with LuGal, a soluble conjugate of luciferin and galactose, and the beta-galactosidase enzyme would hydrolyze this to luciferin. Effluent from each strain would be collected from the microfluidic device and subjected to a luciferase assay for the sensitive detection of luciferin. In some embodiments, a microfluidic device is provided, wherein the microfluidic device comprises one or more colonies or cultures of microorganism cells at one or more predetermined addressable locations, wherein each of the cells within the one or more colonies or cultures comprises an expression cassette comprising a biosensor or promoter operably linked to a polynucleotide encoding a detectable agent, wherein transcription of the biosensor or promoter is modulated by the presence of an analyte. In some embodiments, the detectable agent is a nucleic acid, detectable protein, antibody-linked reporter protein, enzymatic assay product, or electrochemical reaction product. In some embodiments, the detectable agent is an enzymatic assay product. In some embodiments, the enzymatic assay product is beta-galactosidase enzyme. In some embodiments, the detectable agent is detected by addition of X-gal or LuGal.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

Recombinant expression technology typically involves the use of a recombinant expression vector comprising an expression cassette and a mammalian host cell comprising the recombinant expression vector with the expression cassette or at least the expression cassette, which may for example, be integrated into the host cell genome.

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (See, e.g., Goodey, Andrew R.; et al., Peptide and DNA sequences, U.S. Pat. No. 5,302,697; Weiner et al., Compositions and methods for protein secretion, U.S. Pat. Nos. 6,022,952 and 6,335,178; Uemura et al., Protein expression vector and utilization thereof, U.S. Pat. No. 7,029,909; Ruben et al., 27 human secreted proteins, US 2003/0104400 A1). For expression of multi-subunit proteins of interest, separate expression vectors in suitable numbers and proportions, each containing a coding sequence for each of the different subunit monomers, can be used to transform a host cell. In other embodiments, a single expression vector can be used to express the different subunits of the protein of interest.

The term “host cell” means a cell that has been transformed, or is capable of being transformed, with a nucleic acid and thereby expresses a gene or coding sequence of interest. The term includes the progeny of the parent cell, whether or not the progeny is identical in morphology or in genetic make-up to the original parent cell, so long as the gene of interest is present. Any of a large number of available and well-known host cells may be used in the practice of this invention to obtain the antigen-binding proteins of the invention, including mammalian cells, insect cells, microbial cells, or plant cells. For some embodiments mammalian host cells capable of post-translationally glycosylating antibodies may be preferred by the skilled artisan. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Modifications can be made at the DNA level, as well. The peptide-encoding DNA sequence may be changed to codons more compatible with the chosen host cell. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

Within these general guidelines, microbial host cells in culture, such as bacteria (such as Escherichia coli sp.), and yeast cell lines (e.g., Saccharomyces, Pichia, Schizosaccharomyces, Kluyveromyces) and other fungal cells, algal or algal-like cells, insect cells, plant cells, that have been modified to incorporate humanized glycosylation pathways, can also be used to produce fully functional glycosylated antibody. However, mammalian (including human) host cells, e.g., CHO cells and HEK-293 cells, are also useful.

Examples of useful mammalian host cell lines are Chinese hamster ovary cells, including CHO-K1 cells (e.g., ATCC CCL61), CHO-S, DXB-11, DG-44, and Chinese hamster ovary cells/-DHFR (CHO, Urlaub et al, Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture (Graham et al, J. Gen Virol. 36: 59 (1977)); baby hamster kidney cells (BHK, ATCC CCL 10); mouse Sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68 (1982)); MRC 5 cells or FS4 cells; or mammalian myeloma cells, e.g., NS0 or sp2/0 mouse myeloma cells. “Cell,” “cell line,” and “cell culture” are often used interchangeably and all such designations herein include cellular progeny. For example, a cell “derived” from a CHO cell is a cellular progeny of a Chinese Hamster Ovary cell, which may be removed from the original primary cell parent by any number of generations, and which can also include a transformant progeny cell. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Host cells are transformed or transfected with the above-described nucleic acids or vectors for production of polypeptides (including antigen binding proteins, such as antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker or reporter are particularly useful for the expression of polypeptides, such as antibodies.

A “fixed cell” is a cell that is preserved, in a state with cellular structures stabilized closely to “life-like” positions, adequate for the intended purpose, e.g., use in an assay or in an imaging protocol. The process of fixation, which kills the cells, commonly involves a “fixative” chemical, such as formaldehyde or paraformaldehyde (PFA, i.e., polymeric formaldehyde) dissolved in water or a buffer, which works by chemically bonding together (by covalent cross-linkage) adjacent macromolecules, such as proteins. The free methanediols in the PFA solution are reactive with amine groups on proteins and other cellular structures that contain nitrogen. PFA also solubilizes some lipids in cellular membranes. PFA is commonly diluted to 3.7-5% (v/v) and is applied to cells for about 10-15 minutes. While formaldehyde (or PFA) has broad reactivity with a majority of proteins, peptides, and enzymes and is the most commonly used fixative, other approaches can be used to fix cells; for example glutaraldehyde can be used as a stronger crosslinking fixative, or glutaraldehyde can be used in combination with formaldehyde (or PFA). Cold alcohol fixation is sometimes used an alternative, especially fixing for membrane-surface antigens.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

The host cells can be usefully grown in batch culture, fed-batch culture, intensified fed-batch culture (product retention perfusion), or in continuous culture systems employing liquid aqueous medium. Mammalian cells, such as CHO and BHK cells, are generally cultured as suspension cultures. That is to say, the cells are suspended in a liquid cell culture medium, rather than adhering to a solid support. In other embodiments, the mammalian host cells can be cultured on solid or semi-solid aqueous culture medium, for example, containing agar or agarose, to form a medium, carrier (or microcarrier) or substrate surface to which the cells adhere and form an adhesion layer. Another useful mode of production is a hollow fiber bioreactor with an adherent cell line. Porous microcarriers can be suitable and are available commercially, sold under brands, such as Cytoline®, Cytopore® or Cytodex® (GE Healthcare Biosciences).

“Cell culture medium” or “culture medium,” used interchangeably, is defined, for purposes of the invention, as a sterile medium suitable for growth of cells, and preferably animal cells, more preferably mammalian cells (e.g., CHO cells), in in vitro cell culture. Any medium capable of supporting growth of the appropriate cells in cell culture can be used. Suitably, the culture medium has an osmolality of between 210 and 650 mOsm, preferably 270 to 450 mOsm, more preferably 310 to 350 mOsm and most preferably 320 mOsm. Preferably, the osmolality of the cell culture supernatant is maintained within one or more of these ranges throughout the culturing of host cells. The cell culture medium can be based on any basal medium such as DMEM, or RPMI generally known to the skilled worker. Commercially available media such as ExpiCHO™ Expression Medium (ThermoFisher Scientific), Ham's F10 (Sigma), Ham's F12, Medium 199, McCoy, Minimal Essential Medium ((MEM), (Sigma-Aldrich), RPMI-1640 (Sigma), and Dulbecco's Modified Eagle's Medium ((DMEM), Sigma-Aldrich) are suitable for culturing various host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. patent Re. No. 30,985 may be used as culture media for the host cells, or modified appropriately to suit the cell line employed. Other examples include HyClone ActiPro™ and Lonza PowerCHO-2™. The basal medium can comprise a number of ingredients, including amino acids, vitamins, organic and inorganic salts, and sources of carbohydrate, each ingredient being present in an amount which supports the cultivation of a cell which is generally known to the person skilled in the art. The medium can contain auxiliary substances, such as buffer substances like sodium bicarbonate, antioxidants, stabilizers to counteract mechanical stress, or protease inhibitors. Any of these media may be supplemented as necessary with hormones and/or other growth factors (preferably recombinantly produced), such as insulin, insulin-like growth factor (IGF)-1, transferrin, or epidermal growth factor; salts, such as sodium chloride, calcium, magnesium, and phosphate; buffers, such as HEPES and/or sodium bicarbonate; nucleotides, such as adenosine and thymidine; antibiotics, such as gentamicin, neomycin, tetracycline, puromycin, or kanamycin; trace elements (defined as inorganic compounds usually present at final concentrations in the micromolar range); and glucose or an equivalent carbon and/or energy source, such that the physiological conditions of the cell in, or on, the medium promote expression of the protein of interest by the host cell; any other necessary supplements may also be included at appropriate concentrations that would be known to those skilled in the art.

Historically, mammalian cells have been cultured in media containing mammalian serum. The culture medium can include a suitable amount of serum such a fetal bovine serum (FBS). The term “serum-comprising” as applied to cell culture medium includes any cell culture medium that does contain serum. However, such media are incompletely defined and carry the risk of infection, therefore, preferably, the host cells can be adapted for culture in serum-free medium. The term “serum-free” as applied to medium includes any cell culture medium that does not contain serum. By “serum-free”, it is understood that the medium has preferably less than 0.1% (v/v) serum and more preferably less than 0.01% (v/v) serum. The term “serum” refers to the fluid portion of the blood obtained after removal of the fibrin clot and blood cells.

Those in the art have devised “protein-free” media that are either completely free of any protein or at least are free of any protein that is not recombinantly produced. For example, due to the labile nature of Factor VIII (FVIII), the productivity of the engineered host cells is severely reduced under protein-free conditions. Human serum albumin is commonly used as a serum-free culture supplement for the production of recombinant proteins. The albumin itself stabilizes the FVIII, and the impurities present in serum-derived albumin preparations may also contribute to the stabilizing effect of albumin. Factors such as lipoprotein have been identified as a replacement for human serum albumin for the production of recombinant Factor VIII (FVIII), under serum-free conditions. Useful cell culture media include those disclosed in U.S. Pat. No. 6,171,825 (Chan et al., Preparation of recombinant factor VIII in a protein free medium, Bayer, Inc.) and U.S. Pat. No. 6,936,441 (Reiter et al., Recombinant cell clones having increased stability and methods of making and using the same, Baxter AG). The medium of U.S. Pat. No. 6,171,825 (Chan et al.) comprises modified Dulbecco's Minimum Essential Medium and Ham's F-12 Medium (50:50, by weight) supplemented with recombinant insulin, iron, a polyol, copper and optionally other trace metals.

If insulin is used, it should be recombinant and can be obtained commercially as “Nucellin” insulin (Eli Lilly. It can be added at 0.1 to 20 μg/mL (preferably 5-15 μg/mL, or about 10 μg/mL). The iron is preferably in the form of Fe²⁺ ions, for example provided as FeSO₄EDTA, and can be present at 5-100 μM (preferably about 50 μM). Suitable polyols include non-ionic block copolymers of poly(oxyethylene) and poly(oxypropylene) having molecular weights ranging from about 1000 to about 16,000 Da. A particularly preferred polyol is Pluronic F-68 (BASF Wyandotte), which has an average molecular weight of 8400 Da and consists of a center block of poly(oxypropylene) (20% by weight) and blocks of poly(oxyethylene) at both ends. It is also available as Synperonic F-68 from Unichema Chemie BV. Others include Pluronics F-61, F-71 and F-108. Copper (Cu²⁺) may be added in an amount equivalent to 50-800 nM CuSO₄, preferably 100-400 nM, conveniently about 250 nM. The inclusion of a panel of trace metals such as manganese, molybdenum, silicon, lithium and chromium can lead to further increases in Factor VIII production. BHK cells grow well in this protein-free basal medium.

The medium of U.S. Pat. No. 6,936,441 (Reiter et al.) is particularly well suited to the culturing of CHO cells but may be used with other cells as well. The medium of U.S. Pat. No. 6,936,441 is also based on a 50/50 mixture of DMEM and Ham's F12 but includes soybean peptone hydrolysate or yeast extract at between 0.1 and 100 g/L, preferably between 1 and 5 g/L. As a particularly preferred embodiment, soybean extract, e.g. soybean peptone, may be used. The molecular weight of the soybean peptone can be less than 50 kDa, preferably less than 10 kDa. The addition of ultrafiltered soybean peptone having an average molecular weight of 350 Da has proven particularly advantageous for the productivity of the recombinant cell lines. It is a soybean isolate having a total nitrogen content of about 9.5% and a free amino acid content of about 13%, or about 7-10%.

Another useful embodiment of a cell culture medium has the following composition: synthetic minimum medium (e.g. 50/50 DMEM/Ham's F12) 1 to 25 g/L; soybean peptone 0.5 to 50 g/L; L-glutamine 0.05 to 1 g/L; NaHCO₃ 0.1 to 10 g/L; ascorbic acid 0.0005 to 0.05 g/L; ethanolamine 0.0005 to 0.05; and sodium selenite 1 to 15 μg/L. Optionally, a “defoaming” or “anti-foaming” agent can be added to the culture medium. Examples include, a silicone antifoam agent, or a non-ionic surface-active agent such as a polypropylene glycol (e.g. Pluronic F-61, Pluronic F-68, Pluronic F-71 or Pluronic F-108). Another example of a useful commercially available anti-foaming agent is Ex-Cell© Antifoam (Sigma-Aldrich, Inc., St. Louis, MO; Product No. 59920C). The anti-foam agent is generally applied to protect the cells from the negative effects of aeration (“sparging”), since without the addition of a surface-active agent the rising and bursting air bubbles may damage those cells that are at the surface of the air bubbles.

The amount of non-ionic surface-active agent can range between 0.05 and 10 g/L, preferably between 0.1 and 5 g/L. Furthermore, the medium can also contain cyclodextrin or a derivative thereof. The serum- and protein-free medium can also contain a protease inhibitor, such as a serine protease inhibitor, which is suitable for tissue culture and which is of synthetic or vegetable origin. Non-ionic surfactants or antifoaming agents, if present in the cell culture medium, are preferably removed from the buffer in which the antibodies are dissolved before any affinity chromatography steps, lest they interfere.

In another embodiment of a cell culture medium, the following amino acid mixture is can be added to the above-mentioned medium: L-asparagine (0.001 to 1 g/L; preferably 0.01 to 0.05 g/L; particularly preferably 0.015 to 0.03 g/l), L-cysteine (0.001 to 1 g/L; preferably 0.01 to 0.05 g/L; particularly preferably 0.015 to 0.03 g/L), L-proline (0.001 to 1.5 g/L; preferably 0.01 to 0.07 g/L; particularly preferably 0.02 to 0.05 g/L), L-tryptophan (0.001 to 1 g/L; preferably 0.01 to 0.05 g/L; particularly preferably 0.015 to 0.03 g/L) and L-glutamine (0.05 to 10 g/L; preferably 0.1 to 1 g/L). These amino acids may be added to the medium individually or in combination. The combined addition of the amino acid mixture containing all of the above-mentioned amino acids is particularly preferred.

In one embodiment, a serum- and protein-free medium is used additionally containing a combination of the above-mentioned amino acid mixtures and purified, ultrafiltered soybean peptone hydrolysate.

Nutrient supplements such as yeast hydrolysate or various plant-based hydrolysates can be included in the medium, if desired. In some embodiments, the aqueous medium is liquid, such that the host cells are cultured in a cell suspension within the liquid medium. Alternate media capable of supporting CHO cell growth and productivity of antibody can be used interchangeably with the media used in the working example described herein. The possibilities are numerous and could include commercial media made by Sigma-Aldrich, Sartorius or Irvine Scientific, as well as, media especially formulated for a variety of suitable host cell types.

The term “hydrolysate” includes any digest of an animal derived or plant derived source material, or extracts derived from yeast, bacteria, or plants, e.g., “soy hydrolysate,” which can be a highly purified soy hydrolysate, a purified soy hydrolysate or crude soy hydrolysate.

A further suitable cell culture medium is the oligopeptide-free medium disclosed in US 2007/0212770 A1 (Grillberger et al., Oligopeptide-free cell culture media; Baxter International Inc., Baxter Healthcare S.A.), but any suitable cell culture medium that provides physiological conditions permitting the expression of antibody proteins by the host cells can be employed, including other media described in the Examples herein.

The term “inoculation of the cells into the cell culture medium” refers to the step of contacting the cells with the cell culture medium under conditions which are suitable for growth and proliferation of the cells.

The cell culture contemplated herein may be any cell culture independently of the kind and nature of the cultured cells and the growth phase of the cultured cells, e.g. adherent or non-adherent cells; growing, or growth-arrested cells.

The term “sterile,” as used herein, refers to a substance that is free, or essentially free, of microbial and/or viral contamination. In this respect the “contaminant” means a material that is different from the desired components in a preparation being a cell culture medium or at least a component of a cell culture medium. In the context of “sterile filtration”, the term sterile filtration is a functional description that a preparation is filtered through a sterile filter (with a pore size of 0.2 μm or less) to remove bacterial and/or mycoplasma contaminants.

The “cell culture supernatant” is the extracellular medium in which the mammalian cells are cultured. This medium is not to be confused with feed medium that may be added to the culture after inoculation of the cells into the cell culture medium and cell growth has been commenced. A “cell culture” means the cell culture supernatant and the mammalian cells cultured therein. Conventionally, mammalian cells are cultured at 37 °C±1 °C.

“Culturing at” or “maintaining at” a set point of a particular desired temperature, means that the process control systems are set to that desired temperature, in other words that the set point of temperature is the intended target temperature. The culture conditions, such as temperature (typically, but not necessarily, about 37 °C), pH (typically, but not necessarily, a cell culture medium is maintained within the range of about pH 6.5-7.5, as modified consistent with the present invention), oxygenation, and the like, will be apparent to the ordinarily skilled artisan. Clearly, there will be small variations of the temperature of a culture over time, and from location to location through the culture vessel. Digital control units and sensory monitors are available commercially or can be constructed by the skilled artisan. Alternative digital control units (DCU) control and monitor the cell culture process are available commercially, made by companies such as B. Braun, New Brunswick, or Sartorius. For in-flask batch culture with shaker, numerous models of suitable cell culture incubators with built-in environmental controls (e.g., CO₂ and Multigas CO₂/O₂ controls) are commercially available, e.g., by Thermo Fisher Scientific.

“Culturing at” or “maintaining at” a temperature that is set at X±Y°C, means that the set point is at a value of from X+Y°C to X-Y°C. For example, where X and Y are 37.0 and 0.9, respectively, the set point is set at a value of from 37.9 to 36.1 °C. For each of the preferred values of X, e.g., X=31, X=32, X=33, X=34, X=35, X=36, or X=37, the set-point is at a value within the range X±0.9 °C, ±0.8 °C, ±0.7 °C, ±0.6 °C, ±0.5 °C, ±0.4 °C, ±0.3 °C, ±0.2 °C, or ±0.1 °C. (See, e.g., Oguchi et al., pH Condition in temperature shift cultivation enhances cell longevity and specific hMab productivity in CHO culture, Cytotechnology. 52(3):199-207 (2006); Al-Fageeh et al., The cold-shock response in cultured mammalian cells: Harnessing the response for the improvement of recombinant protein production, Biotechnol. Bioeng. 93:829-835 (2006); Marchant, R. J. et al., Metabolic rates, growth phase, and mRNA levels influence cell-specific antibody production levels from in vitro cultured mammalian cells at sub-physiological temperatures, Mol. Biotechnol. 39:69-77 (2008)).

For any given set-point, slight variations in temperature may occur. Typically, such variation may occur because heating and cooling elements are only activated after the temperature has deviated somewhat from the set-point. In that case, the set-point is X±Y and the heating or cooling element is activated when the temperature varies by ±Z°C, as appropriate. Typically, the permissible degree of deviation of the temperature from the set-point before heating or cooling elements are activated may be programmed in the process control system. Temperature may be controlled to the nearest ±0.5 °C, ±0.4 °C, ±0.3 °C, ±0.2 °C, or even ±0.1 °C by heating and cooling elements controlled by thermostats. Larger differentials in temperature may also be programmed, such as ±1.0 °C, ±0.9 °C, ±0.8 °C, ±0.7 °C, or ±0.6 °C. The temperature may also be controlled by immersion of the culture vessel in a heating bath at a particular temperature. Conceivably, there is no variation from the set-point because the heating is applied continually, which can also involve variable heating output in the case of variable ambient temperature. Another source of variation arises due to measurement error in the temperature of the cell culture supernatant. Typical thermometers used in cell culture equipment may have a variability of ±0.3 °C, or ±0.2 °C, or even ±0.1 °C.

Where the temperature set-point is set at a value within the range X±Y°C, and the tolerance of the temperature is ±Z°C (i.e. a heater or cooler is activated when the temperature deviates by ±Z°C, as appropriate) this can also be expressed as a set-point of (X−Y to X+Y)±Z°C. For each possible value of X, all combinations of ±Y°C and ±Z°C, as indicated above, are envisaged.

“Culturing at” or “maintaining at” a set point of a particular desired pH value, means that the process control systems are set to that desired pH value, in other words that the set point of pH is the intended target pH. “Culturing at” or “maintaining at” a pH that is set at X±Y, means that the set point is at a value of from X+Y to X-Y pH units. For each of the preferred values of X, the set-point is at a value within the range pH X±0.05, ±0.04, ±0.03, ±0.02 or ±0.01.

Where the pH set-point is set at a value within the range X±Y, and the tolerance is ±Z, this can also be expressed as a set-point of (X−Y to X+Y)±Z. For each possible value of X, all combinations of ±Y and ±Z, as indicated above.

For any given pH set-point, slight variations in pH may occur. Typically, such variation can occur because means which control pH are only activated after the pH has deviated somewhat from the set-point. Typically, the pH is controlled to the nearest ±0.05, ±0.04, ±0.03, ±0.02, or ±0.01. Typically, sparging with CO₂ provides additional acid in mammalian cell culture. Liquid acids, e.g., HCl or H₃PO₄, are commonly used in microbial cultures. Sodium carbonate is usually the source of added alkali used to maintain pH for mammalian cell culture, and NH₄OH is often selected to add alkali in microbial culture.

The cell culture supernatant typically has a CO₂ concentration of 1 to 10% (v/v), for example 4.0-9.0% (v/v), 5.5-8.5% (v/v) or about 6-8% (v/v). Conventionally, CO₂ concentration is higher than this due to the CO₂ produced by the cells not being removed from the cell culture supernatant. Maintaining the CO₂ concentration at 10% or lower is reported to increase the yield of recombinant protein; it helps the dCO2 (or pCO₂) to be kept low if the feed medium is degassed (for example by bubbling air through it) as well as the cell culture supernatant in the bioreactor being sparged. (See, Giovagnoli et al., Cell Culture Processes, US2009/0176269, US2016/0244506, U.S. Pat. No. 9,359,629, EP2235197, EP2574676).

Ways of monitoring culture parameters of temperature, pH and CO₂ concentration are well known in this art and generally rely on probes that are inserted into the bioreactor, or included in loops through which the culture medium is circulated, or inserted into extracted samples of culture medium. Suitable monitoring equipment and appropriate alternatives are commercially available or can be constructed by the skilled artisan. Alternative gas analyzers are commercially available, such as RapidLab® 248 (Siemens) and others made by Nova® Biomedical, Radiometer America and Roche Diagnostics. Mass flow controllers can also be used to control gas and liquid additions in labs that are properly equipped. A suitable in-line dCO₂ (or pCO2) sensor and its use are described in Pattison et al (2000) Biotechnol. Prog. 16:769-774. A suitable in-line pH sensor is Mettler Toledo InPro 3100/125/Pt100 (Mettler-Toledo Ingold, Inc., Bedford, Mass.). A suitable off-line system for measuring dCO₂ (or pCO2), in addition to pH and pO₂ is the BioProfile pHOx (Nova Biomedical Corporation, Waltham MA). In this system, or dCO₂ (or pCO₂) is measured by potentiometric electrodes within the range 3-200 mmHg with an imprecision resolution of 5%. The pH may be measured in this system at a temperature of 37 °C, which is close to the temperature of the cell culture supernatant in the bioreactor. Ways of altering the specified parameter in order to keep it at the predefined level are also well known. For example, keeping the temperature constant usually involves heating or cooling the bioreactor or the feed medium (if it is a fed-batch or continuous process); keeping the pH constant usually involves choosing and supplying enough of an appropriate buffer (typically bicarbonate) and adding acid, such as hydrochloric acid, or alkali, such as sodium hydroxide, sodium carbonate or a mixture thereof, to the feed medium as necessary; and keeping the CO₂ concentration constant usually involves adjusting the sparging rate (see further below), or regulating the flow of CO₂ in the head space. It is possible that the calibration of an in-line pH probe may drift over time, such as over periods of days or weeks, during which the cells are cultured. In that event, it may be beneficial to reset the in-line probe by using measurements obtained from a recently calibrated off-line probe. A suitable off-line probe is the BioProfile pHOx (Nova Biomedical Corporation, Waltham MA).

Mammalian cell cultures, and many other kinds of microbial cells, need oxygen for the cells to grow, or can grow fastest under aerobic conditions. Normally, this is provided by forcing oxygen into the culture through injection ports. It is also necessary to remove the CO₂ that accumulates due to the respiration of the cells. This is achieved by “sparging,” i.e., passing a gas through the bioreactor in order to entrain and flush out the CO₂. Conventionally, this can also be done using oxygen. However, the inventors have found that it is advantageous to use air instead. It has been found that usually a conventional inert gas such as nitrogen is less effective at sparging CO₂ than using air. Given that air is about 20% (v/v) oxygen, one might have thought that five times as much air would be used. However, this has been found to be inadequate in large scale cultures, particularly in cultures at 2500 L scale. In a 2500-L bioreactor, 7 to 10 times as much air, preferably about 9 times as much air, is used. For example, under standard conditions, the 2500-L bioreactor is sparged with O₂ at a 10-μm bubble size at a rate of 0.02 VVH (volume O₂ per volume of culture per hour). The same 2500-L bioreactor used according to the method of the invention would be sparged with air at a 10-μm bubble size at a rate of 0.18 VVH.

Hence, the use of surprisingly high volumes of air has been found to provide adequate oxygen supply and to remove the unwanted CO₂. Flushing the bioreactor head space with air is also a useful mechanism for removing excess CO₂.

During production phase, it is preferred to remove CO₂ by air sparging, as described above. This is especially the case when using bioreactors of large capacity, in which the cell culture supernatant would otherwise accumulate CO₂ to deleteriously high levels. However, at the beginning of culture, or in small scale culture, such as at 1-L or 2.5-L scale, the head space may be overlayed with CO₂. Under such conditions, low levels of dCO₂ (or pCO₂) can still be achieved. Overlaying the headspace with CO₂ may also be used to reduce the pH to the set-point, if the pH is too basic.

In accordance with inventive method, the culturing of a plurality of mammalian host cells can be any conventional type of culture, such as batch, fed-batch, intensified fed-batch, or continuous. Suitable continuous cultures included repeated batch, chemostat, turbidostat or perfusion culture. For purposes of the present invention, the desired scale of the recombinant expression will be dependent on the type of expression system and the quantity of different theoretical antibody variants to be studied. As noted herein, typically, 100 milligrams of total antibody protein will suffice, requiring only a batch cell culture of 20 mL to 500 mL; while larger scale culture batches or continuous cell culture methods can be employed, larger volumes are typically not cost-effective.

A batch culture starts with all the nutrients and cells that are needed, and the culture proceeds to completion, i.e. until the nutrients are exhausted or the culture is stopped for some reason.

A fed-batch culture is a batch process in the sense that it starts with the cells and nutrients but it is then fed with further nutrients in a controlled way. The fed-batch strategy is typically used in bio-industrial processes to reach a high cell density in the bioreactor. The feed solution is usually highly concentrated to avoid dilution of the bioreactor. The controlled addition of the nutrient directly affects the growth rate of the culture and allows one to avoid overflow metabolism (formation of metabolic by-products) and oxygen limitation (anaerobiosis). In most cases the growth-limiting nutrient is glucose which is fed to the culture as a highly concentrated glucose syrup (for example 500-850 g/L).

Different strategies can be used to control the growth in a fed-batch process. For example, any of dissolved oxygen tension (DOT, pO2), oxygen uptake rate (OUR), glucose concentration, lactate concentration, pH and ammonia concentration can be used to monitor and control the culture growth by keeping that parameter constant. In a continuous culture, nutrients are added and, typically, medium is extracted in order to remove unwanted by-products and maintain a steady state. Suitable continuous culture methods are repeated batch culture, chemostat, turbidostat and perfusion culture.

CHO cells, for example, may be cultured in a stirred tank or an airlift tank that is perfused with a suitable medium at a perfusion rate of from 1 to 10 volume exchanges per day and at an oxygen concentration of between 40% and 60%, preferably about 50%. Moreover, the cells may be cultured by means of the chemostat method, using the preferred pH value given above, an oxygen concentration of between 10% and 60% (preferably about 20%) and a dilution rate D of 0.25 to 1.0, preferably about 0.5.

In a repeated batch culture, also known as serial subculture, the cells are placed in a culture medium and grown to a desired cell density. To avoid the onset of a decline phase and cell death, the culture is diluted with complete growth medium before the cells reach their maximum concentration. The amount and frequency of dilution varies widely and depends on the growth characteristics of the cell line and convenience of the culture process. The process can be repeated as many times as required and, unless cells and medium are discarded at subculture, the volume of culture will increase stepwise as each dilution is made. The increasing volume may be handled by having a reactor of sufficient size to allow dilutions within the vessel or by dividing the diluted culture into several vessels. The rationale of this type of culture is to maintain the cells in an exponentially growing state. Serial subculture is characterized in that the volume of culture is always increasing stepwise, there can be multiple harvests, the cells continue to grow and the process can continue for as long as desired.

In the chemostat and turbidostat methods, the extracted medium contains cells. Thus, the cells remaining in the cell culture vessel must grow to maintain a steady state. In the chemostat method, the growth rate is typically controlled by controlling the dilution rate i.e. the rate at which fresh medium is added. The cells are cultured at a sub-maximal growth rate, which is achieved by restricting the dilution rate. The growth rate is typically high. In contrast, in the turbidostat method, the dilution rate is set to permit the maximum growth rate that the cells can achieve at the given operating conditions, such as pH and temperature.

In an intensified fed-batch culture, culture vessels, reactors or chambers, of any of various capacities are used to grow suspensions of mammalian host cells, e.g., CHO cells. Each culture vessel is connected via inlets to an array of porous tangential flow filters which in turn are connected via outlets back to the culture vessel. After cell growth, the suspensions of host cells and growth medium are pumped through the array of porous tangential flow filters to concentrate the cell suspension. The cell suspension is recycled through the filters and culture vessel allowing a portion of the old growth medium (and its serum components, if any) to be removed. A supply of fresh sterile serum-free expression medium is added to the concentrated cell suspension to maintain a nominal volume in the culture vessel. The recombinant protein of interest, e.g., an antibody, is produced subsequently by the host cells suspended in the expression medium and is secreted by the cells into the expression medium from which it can be harvested by standard techniques. (See, e.g., Zijlstra et al., process for the culturing of cells, U.S. Pat. Nos. 8,119,368, 8,222,001, 8,440,458).

In a perfusion or continuous culture, the extracted medium is depleted of cells, because most of the cells are retained in the culture vessel, for example, by being retained on a membrane through which the extracted medium flows. However, typically such a membrane retains 100% of cells, and so a proportion are removed when the medium is extracted. Alternatively, sonic cell separation technology achieves separation of cells from the media matrix with high-frequency, resonant ultrasonic waves rather than using a physical barrier, unlike tangential-flow filtration (TFF) or alternating tangential flow filtration (ATF); the cells are held back using an acoustic field as the bioprocess fluid flows through an open channel. The use of acoustic waves allows differentiation of particles of equal size, and thus the technology can be used for the separation of particles from the nano- to macro-scales. (See, e.g., Challenger, C. A., An acoustic wave-based technology for cell harvesting applications may help enable continuous manufacturing, BioPharm International 30(9):30 (2017)). Regardless of the technology employed to separate the cells from the extracted medium, it may not be crucial to operate perfusion cultures at very high growth rates, as the majority of the cells are retained in the culture vessel.

Continuous cultures, particularly repeated batch, chemostat and turbidostat cultures, are typically operated at high growth rates. According to common practice, it is typical to seek to maintain growth rates at maximum, or close to maximum, in an effort to obtain maximum volumetric productivity. Volumetric productivity is measured in units of protein quantity or activity per volume of culture per time interval. Higher cell growth equates to a higher volume of culture being produced per day and so is conventionally considered to reflect a higher volumetric productivity. A suitable fully continuous process can have a perfusion bioreactor coupled to recombinant protein harvesting and protein purification steps, for example, a multi-column chromatography capture step, followed by flow-through virus inactivation, multi-column intermediate purification, a flow-through membrane adsorber polishing step, continuous virus filtration and a final ultrafiltration step operated in continuous mode. (See, e.g., Crowley et al., Process for cell culturing by continuous perfusion and alternating tangential flow, U.S. Pat. No. 8,206,981).

The cell density is commonly monitored in cell cultures. In principle, a high cell density would be considered to be desirable since, provided that the productivity per cell is maintained, this should lead to a higher productivity per bioreactor volume. However, increasing the cell density can actually be harmful to the cells, and the productivity per cell is reduced. There is therefore a need to monitor cell density. To date, in mammalian cell culture processes, this has been done by extracting samples of the culture and analyzing them under a microscope or using a cell counting device such as the CASY TT device sold by Scharfe System GmbH, Reutlingen, Germany. It can be advantageous to analyze the cell density by means of a suitable probe introduced into the bioreactor itself (or into a loop through which the medium and suspended cells are passed and then returned to the bioreactor). Such probes are available commercially from Aber Instruments, for example the Biomass Monitor 220, 210 220 or 230. The cells in the culture act as tiny capacitors under the influence of an electric field, since the non-conducting cell membrane allows a build-up of charge. The resulting capacitance can be measured; it is dependent upon the cell type and is directly proportional to the concentration of viable cells. A probe of 10 to 25 mm diameter uses two electrodes to apply a radio frequency field to the biomass and a second pair of electrodes to measure the resulting capacitance of the polarized cells. Electronic processing of the resulting signal produces an output which is an accurate measurement of the concentration of viable cells. The system is insensitive to cells with leaky membranes, the medium, gas bubbles and debris. Alternatively, cell viability can be measured by use of a vital dye (or vital stain) to stain small-aliquot samples of culture sampled periodically, and microscopically enumerated to determine viable cell count. For example Trypan blue is a vital dye commonly used for this purpose. Automated cell counters supplied by Beckman (e.g., Vi-Cell™ XR) and other companies are available. Examples include cell counting instruments made by other manufacturers, e.g., Nova Biomedical, Olympus, Thermo Fisher Scientific and Eppendorf. Cells can also be counted using flow cytometry or manually by using a hemocytometer.

Typically, a viable cell density can be used from 1.0×10⁶ to 2.0×10⁷, or up to about 5×10⁷ cells/mL. It is known that increasing the concentration of cells toward the higher end of the preferred ranges can improve volumetric productivity. Nevertheless, ranges of cell density including any of the above point values as lower or higher ends of a range are envisaged.

The culture is typically carried out in a bioreactor, which is usually a stainless steel, glass or plastic vessel of 0.01 (i.e., 10-mL) to 10000 (ten thousand) litres capacity, for example, 0.01, 0.015, 0.10, 0.25, 0.30, 0.35, 1, 2, 5, 10, 15, 20, 25, 30, 50, 75, 100, 500, 1000, 2500, 5000 or 8000 liters. The vessel is usually rigid but flexible plastic bags or bioreactor liners can be used. These flexible plastic bioreactor bags and liners are generally of the “single use” type.

Upon culturing the host cells, the recombinant polypeptide or protein, can be produced intracellularly, in the periplasmic space, or, preferably, directly secreted into the medium. Harvesting the recombinant protein involves separating it from particulate matter that can include host cells, cell aggregates, and/or lysed cell fragments, into a cell-free supernatant fraction that is free of host cells and cellular debris. Such cellular debris is removed, for example, by centrifugation or microfiltration. After the recombinant protein, e.g., recombinant antibodies, is separated from the host cells and/or other particulate debris, harvesting the recombinant protein into a cell-free supernatant fraction can optionally involve capture of the recombinant protein by one or more chromatographic capture steps that can partially purify and/or concentrate the protein, such as Protein A or Protein G or Protein L affinity chromatography. (See, e.g., Frank, M. B., “Antibody Binding to Protein A and Protein G beads” 5. In: Frank, M. B., ed., Molecular Biology Protocols. Oklahoma City (1997)).

After harvesting the cell culture fluid comprising a recombinant protein of interest, e.g., an antibody or antibody fragment, can be further purified from the cell-free supernatant fraction. Typically, the purification of recombinant proteins is usually accomplished by an optional series of chromatographic steps such as anion exchange chromatography, cation exchange chromatography, affinity chromatography (using Protein A or Protein G or Protein L as an affinity ligand), hydrophobic interaction chromatography, hydroxy apatite chromatography and size exclusion chromatography. Further, the purification process may comprise one or more ultra-, nano- or diafiltration steps, and/or, optionally, an acidic viral inactivation step. Other optional known techniques for protein purification such as ethanol precipitation, Reverse Phase HPLC, chromatofocusing, SDS-PAGE, and ammonium sulfate precipitation are also possible depending on the antibody to be recovered.

The present method involves harvesting the recombinant antibodies present in the culture supernatant and then purifying the cell-free supernatant fraction by affinity chromatography to purify the antigen-binding protein present in the cell-free supernatant fraction. In this step, affinity chromatography involves loading the cell-free supernatant fraction onto an affinity chromatography matrix having conjugated moieties with particular affinity for immunoglobulin molecules that may be of interest, for example a matrix having a covalently conjugated target molecule (e.g., fentanyl or carfentanil). In embodiments including an immunoglobulin Fc domain, such as some fusion proteins (e.g., peptibodies), such conjugated moieties can include, e.g., Protein A, and/or Protein G, and/or Protein L, or anti-kappa antibodies with an affinity for Fab antibody fragments, or anti-his antibodies, or glutathione, or another suitable matrix-conjugated antibody that specifically binds an immunoglobulin epitope of interest. For example, a Protein A matrix can be used to purify proteins that include polypeptides based on human γ1, γ2, or γ4 heavy chains (Lindmark et al., J. Immunol. Meth. 62: 1-13 (1983)). Also useful in purifying embodiments (e.g., fusion proteins) comprising an immunoglobulin Fc domain are engineered versions of Protein A that are multimers (typically tetramers, pentamers or hexamers) of a single domain which has been modified to improve its characteristics for industrial applications. “Protein A” is an approximately 42 kDa surface protein originally found in the cell wall of the bacteria Staphylococcus aureus; Protein A is encoded by the spa gene of S. aureus, and its expression in S. aureus is controlled by DNA topology, cellular osmolarity, and a two-component system called ArlS-ArlR. (See, Fournier, B., and Klier, A, Protein A gene expression is regulated by DNA supercoiling which is modified by the ArlS-ArlR two-component system of Staphylococcus aureus, Microbiology 150:3807-19 (2004)). Protein A (Spa gene product) is useful in biochemical research and industry because of its ability to bind immunoglobulins. Protein A is composed of five homologous Ig-binding domains that fold into a three-helix bundle. Each domain is able to bind proteins from many mammalian species, most notably IgGs. It has been shown via crystallographic refinement that the primary binding site for Protein A is on the Fc region, between the C_{H}2 and C_{H}3 domains. (Deisenhofer, J., Crystallographic refinement and atomic models of a human Fc fragment and its complex with fragment B of Protein A from Staphylococcus aureus at 2.9- and 2.8-A resolution, Biochemistry 20 (9): 2361-70 (1981)). In addition, Protein A binds human IgG molecules containing IgG F(ab′)₂ fragments from the human VH3 gene family. (See, Sasso E H, Silverman G J, Mannik M, Human IgA and IgG F(ab′)₂ that bind to staphylococcal Protein A belong to the VHIII subgroup, Journal of Immunology. 147 (6): 1877-83 (1991)). Protein A is typically produced and purified in industrial fermentation for use in immunology, biological research and industrial applications. Natural (or native) Protein A can be cultured in Staphylococcus aureus and contains the five homologous antibody binding regions described above and a C-terminal region for cell wall attachment. Recombinant versions of Protein A, typically produced in Escherichia coli, are also useful for purposes of the invention. For use in the present invention, Protein A matrix can be obtained commercially in various embodiments (e.g., Protein A-Sepharose® from Staphylococcus aureus, from Sigma-Aldrich; MabSelect™ Protein A, MabSelect SuRe® Protein A, MabSelect SuRe® LX, and Protein A Sepharose® FF from GE Healthcare Life Sciences; Eshmuno® A Protein A from EMD Millipore; Toyopearl® AF-rProtein A from Tosoh Bioscience; POROS® Protein A from Thermo Fisher Scientific; CaptivA® Protein A affinity resin from Repligen). Recombinant versions of Protein A commonly contain the five homologous antibody binding domains, but for purposes of the present invention can vary in other parts of the structure in order to facilitate covalent coupling to substrates, e.g., resins (such as, but not limited to, agarose). Protein G is recommended for all mouse isotypes and for human γ3 (Guss et al, EMBO J. 5: 15671575 (1986)). Also available commercially (e.g., from Molecular Cloning Laboratories (MCLAB) or Protein Specialists (Prospec)), is recombinant Protein G, an immunoglobulin-binding protein derived from the cell wall of certain strains of beta-hemolytic streptococci. It binds with high affinity to the Fc portion of various classes and subclasses of immunoglobulins from a variety of species. The albumin and cell surface binding domains have been eliminated from Recombinant Protein G to reduce nonspecific binding, although the Fc binding domain is still present and, therefore, can be used to separate IgG from crude samples. The recombinant Protein G is produced in Escherichia coli using sequence from Streptococcus C1-C2-C3. The Protein G contains 200 amino acids (190-384 and five additional residues not including methionine) having a molecular mass of 21.8 kDa. The Protein-G migrates on SDS-PAGE around 32 kDa.

Encompassed within the term “matrix” are resins, beads, nanoparticles, nanofibers, hydrogels, membranes, and monoliths, or any other physical matrix, bearing a relevant covalently bound chromatographic ligand (e.g., Protein A, Protein G, or other affinity chromatographic ligand, such as a target ligand, an antibody targeting an epitope tag, a charged moiety, or a hydrophobic moiety, etc.) for purposes of the inventive method. The matrix to which the affinity target ligand is attached is most often agarose, but other matrices are available. For example, mechanically stable matrices such as controlled pore glass or poly(styrenedivinyl)benzene allow for faster flow rates and shorter processing times than can be achieved with agarose. Where the protein comprises a C_{H}3 domain, the Bakerbond ABX™ resin (J. T. Baker, Phillipsburg, NJ) is useful for purification. An affinity chromatography matrix may be placed or packed into a column useful for the purification of proteins. Loading of the cell-free supernatant fraction onto the affinity chromatography matrix preferably occurs at about neutral pH.

The specific target ligand of interest can be covalently conjugated to an affinity chromatography matrix, e.g., for nanobody purification purposes. The affinity matrix with the target ligand covalently attached should have sufficient binding capacity to account for the required mass sufficient to be detected by a mass spectrometer, if desired. This can be achieved with appropriately dense conjugation reactive moieties on the matrix (e.g., resin and/or resin bed size in the column). In producing and storing the conjugated affinity chromatography matrix reagent for future use in this step, the stability of a particular conjugated affinity chromatography matrix needs to be considered with regards to the conjugated target ligand itself or the mode by which the ligand is attached to the matrix. Ligand affinity conjugation instability and degradation of the conjugated affinity chromatography matrix reagent during storage can result in decreased antibody yields and/or binding artifacts leading to difficult data analysis or misinterpretation. The practitioner should exercise caution with respect to the appropriate storage conditions and quality control employed to ensure the effective quality of the affinity chromatography matrix before use in the inventive method of producing the antigen-binding protein of interest.

The term “to bind” or “binding” a molecule to an affinity chromatography matrix comprising a covalently-conjugated target moiety, e.g., Protein A or a Protein A matrix, or Protein G or a Protein G matrix, or a particular conjugated target ligand of interest, means exposing the molecule to the affinity chromatography target moiety, under appropriate conditions (e.g., pH and selected salt/buffer composition), such that the molecule is reversibly immobilized in, or on, the affinity chromatography matrix (e.g., a Protein A- or Protein G-conjugated or target ligand-conjugated) by virtue of its binding affinity to the target moiety under those conditions, regardless of the physical mechanism of affinity that may be involved. (See, e.g., Jendeberg, L. et al., The Mechanism of Binding Staphylococcal Protein A to Immunoglobin G Does Not Involve Helix Unwinding, Biochemistry 35(1): 22-31 (1996); Nelson, J. T. et al., Mechanism of Immobilized Protein A Binding to Immunoglobulin G on Nanosensor Array Surfaces, Anal. Chem., 87(16):8186-8193 (2015)).

The term “buffer” or “buffered solution” refers to solutions which resist changes in pH by the action of its conjugate acid-base range. Examples of useful buffers that control pH at ranges of about pH 4 to about pH 8 include phosphate, bicarbonate, acetate, MES, citrate, Tris, bis-tris, histidine, arginine, succinate, citrate, glutamate, and lactate, or a combination of two or more of these, or other mineral acid or organic acid buffers. Salts containing sodium, ammonium, and potassium cations are often used in making a buffered solution.

The term “loading buffer” or “equilibrium buffer” refers to the buffer, and salt or salts, which is mixed with a protein preparation (e.g., a batch or perfusion culture supernatant or filtrate, or an eluant pool containing the antibodies of interest) for loading the protein preparation onto an affinity chromatography matrix, e.g., Protein A- or Protein G-conjugated matrix or a specific target ligand-conjugated affinity chromatography matrix, as the case may be. This buffer is also used to equilibrate the matrix before loading, and to wash after loading the protein.

The term “wash buffer” is used herein to refer to the buffer that is passed over an affinity chromatography matrix, following loading of a protein preparation and prior to elution or after flow-through of the protein of interest. The wash buffer may serve to remove one or more contaminants without substantial elution of the desired protein or can be used to wash out a non-binding protein.

The term “elution buffer” or “eluant” refers to the buffer used to elute the protein of interest (POI) reversibly bound to a matrix. As used herein, the term “solution” refers to either a buffered or a non-buffered solution, including water.

The term “eluting” a molecule (e.g. a desired recombinant protein, such as an antibody of interest, or a contaminant) from an affinity chromatography matrix, means removing the molecule from such material, typically by passing an elution buffer over the affinity chromatography matrix. Eluting a bound protein is typically achieved by increasing the conductivity and/or inducing a pH shift and/or a binding competition. This can be performed either over a linear gradient or a step elution to predetermined conditions. Impurities, particularly HMW species, often bind more tightly than the Nb or other antigen-binding protein product and also can be separated from the main desired fraction by adjusting the elution conditions and pool collection criteria (Yigzaw, Y., et al., (2009) supra; Gagnon, P., et al., (1996) supra; Pabst, T. M., et al., (2009) Journal of Chromatography 1216, 7950-7956). The molecular interaction under consideration dictates the type of elution methods that can be used. Thus, salt can be used to disrupt hydrophobic interactions whereas pH can disrupt ionic and hydrogen binds. Other elution methods besides ionic strength and pH can be used to disrupt the interaction between the antibody and ligand. A peptide specific for the antibody epitope on the target ligand can be used to compete with the on-rate and affinity binding properties of the antibody. A small organic molecule can be used in a similar fashion as a peptide. As part of the screening process for a viable candidate, stress can be applied to the antibody pool prior to binding to the ligand affinity column. Thermal, chemical and/or pH stress can induce a conformational change or denaturation event resulting in aggregation of the antibody which can be removed via precipitation (centrifugation or ultrafiltration) or preparative SEC. This step will remove non-viable candidates from binding to the target affinity matrix. Furthermore, the stress can lead to non-aggregated, non-native antibody material which will have decreased binding affinity to the target resulting in selecting against these poor binders. This can simplify the interpretation of the screening data obtained from employing the inventive method.

The phrase “increasingly stringent buffer conditions” means employing a gradient (a step gradient or a linear gradient) of an increasingly more challenging condition by which antibody variants can be distinguished from each other. Examples include, but are not limited to, a gradient (a step gradient or a linear gradient) of increasing ionic strength (typically with higher conductivity going up to about 40-150 mS), or a pH gradient (a step gradient or a linear gradient) approaching an extreme of lower or higher pH than the initial buffer condition, or a gradient (a step gradient or a linear gradient) of increasing concentration of a molecule that competes for binding to the target ligand, such as but not limited to, a small molecule or an oligopeptide. The term “elution pool” or “eluant pool” means the material eluted from a chromatography matrix, which material includes the recombinant protein of interest, e.g., an antibody of interest.

The term “loading,” with respect to an affinity chromatography matrix, means loading a protein preparation (e.g., a batch or perfusion culture supernatant or filtrate, or an eluant pool containing the protein of interest) onto the affinity chromatography matrix.

The term “washing,” with respect to an affinity chromatography matrix, means passing an appropriate buffer through or over the affinity chromatography matrix.

“Under physiological conditions” with respect to incubating buffers and immunoglobulins, or other binding assay reagents means incubation under conditions of temperature, pH, and ionic strength, that permit a biochemical reaction, such as a non-covalent binding reaction, to occur. Typically, the temperature is at room or ambient temperature up to about 37 °C and at pH 6.5-7.5.

“Physiologically acceptable salt” of a composition of matter, for example a salt of a protein of interest, e.g., a fusion protein, or another immunoglobulin, such as an antibody, or any other protein of interest, or a salt of an amino acid, such as, but not limited to, a lysine, histidine, or proline salt, means any salt, or salts, that are known or later discovered to be pharmaceutically acceptable. Some non-limiting examples of pharmaceutically acceptable salts are: acetate salts; trifluoroacetate salts; hydrohalides, such as hydrochloride (e.g., monohydrochloride or dihydrochloride salts) and hydrobromide salts; sulfate salts; citrate salts; maleate salts; tartrate salts; glycolate salts; gluconate salts; succinate salts; mesylate salts; besylate salts; salts of gallic acid esters (gallic acid is also known as 3,4,5 trihydroxybenzoic acid) such as pentagalloylglucose (PGG) and epigallocatechin gallate (EGCG), salts of cholesteryl sulfate, pamoate salts, tannate salts, and oxalate salts.

A “reaction mixture” is an aqueous mixture containing all the reagents and factors necessary, which under physiological conditions of incubation, permit an in vitro biochemical reaction of interest to occur, such as a covalent or non-covalent binding reaction.

The terms “automated,” “automation-controlled,” or “automatically,” are used interchangeably, in connection with a method, process, system, device, apparatus, such as the of the invention, and refer to computer-control of the implementation or performance of one or more process steps or the operation of a component or a system (e.g., activation or deactivation of a pump(s) and/or valve(s) for microfluidic manipulation of aqueous samples, buffers, and/or reagents, and/or detection equipment and data acquisition), optionally, with attendant feed-back regulation of the process step or operation. Typically, an electronic computerized “controller” or “microcontroller” or “digital control unit,” terms used interchangeably herein, receives input digital signals from one or more sensors or detectors of the physical or chemical parameter to be controlled, and/or from a chronometer or clock, and, in comparison to a predetermined control setpoint (often assigned by the operator using a user interface), the controller determines the necessary output signal required to correct the input value in the direction of the setpoint and issues responsive digital instructions to a system or subsystem.

In some embodiments, the microcontroller is situated within the perimeter of a discrete (but optional) system housing, or, in other embodiments, the microcontroller is outside the perimeter of the optional housing, so long as the controller is able to communicate with the pumps, the valves (if present in a particular embodiment), and the sensors within the system or device, i.e., that the controller can receive measurement signals transmitted from the sensors, and the controller can transmit instructions to all the pumps and optional valves. Transmission and/or reception of such measurement signals or instruction signals, as the case may be, can be via electrical cables, wires, fiber optic cables, or can be via infrared or radio wave transmissions, and/or any other suitable medium. In some embodiments, electrical cabling can be Recommended Standard 232 (RS-232) cabling, and can be configured to accommodate serial, bus, or ethernet communications.

The term “switching,” or “switch,” used herein interchangeably, means to initiate, impel, or cease, the flow of an aqueous fluid, e.g. from one vessel, through a connecting line, to a different vessel. Such switching can be under the automatic control and regulation of a computer, i.e., the controller or microcontroller, and mechanism(s), e.g., pumps and, optionally, valves, and the system can optionally be governed by an additional external control system. Switching can be manually controlled and regulated; however, “automatically switching” means that the switch in flow does not require manual input on the operator's part, but is controlled instead by the controller or microcontroller, which performs the switch, initiating flow or ceasing flow, under predetermined criteria or set-points.

A step of a method or process, or within a system or device, is performed “fluidly,” or is “fluidly connected” to, or “fluidly receives” material from, another step of the process or from another component or vessel within the system, when material flows by pipe, tubing, or other closed conduit between steps or systems without manual loading or unloading. Accordingly, “fluidly connected” means that fluid is able to flow from one component or vessel, to another component or vessel, but the two components or vessels need not be physically connected to one another. The pipe, tubing, or other closed conduit between steps or systems is considered a “connecting line,” if it is capable of fluidly conveying the fluid downstream within the system or between system operations. The connecting line can be made of any suitable non-reactive, non-porous material, such as, but not limited to silicone rubber, Teflon®, stainless steel, or the like. Aseptic connectors are useful to join connecting lines to other components, as the skilled person is aware.

The term “fluid propeller” means any suitable device for impelling fluid flow within a system, vessel, or connecting line, e.g., a pump head, a pump, a gas pressure regulator, a vacuum line, a vacuum pump, hydrostatic pressure, or gravity.

A “stable” formulation, such as one comprising a pharmaceutical composition of the present invention, is one in which the protein therein, e.g., a single domain antibody (or nanobody), essentially retains its physical stability and/or chemical stability and/or biological activity upon processing (e.g., ultrafiltration, diafiltration, other filtering steps, vial filling), transportation, and/or storage of the antibody drug substance and/or drug product. Together, the physical, chemical and biological stability of the protein in a formulation embody the “stability” of the protein formulation, which is specific to the conditions under which the formulated drug product (DP) is stored. For instance, a drug product stored at subzero temperatures would be expected to have no significant change in either chemical, physical or biological activity while a drug product stored at 40 °C would be expected to have changes in its physical, chemical and biological activity with the degree of change dependent on the time of storage for the drug substance or drug product. The configuration of the protein formulation can also influence the rate of change. For instance, aggregate formation is highly influenced by protein concentration with higher rates of aggregation observed with higher protein concentration. Excipients are also known to affect stability of the drug product with, for example, addition of salt increasing the rate of aggregation for some proteins while other excipients such as sucrose are known to decrease the rate of aggregation during storage. Instability is also greatly influenced by pH giving rise to both higher and lower rates of degradation depending on the type of modification and pH dependence.

Various analytical techniques for measuring protein stability are available in the art and are reviewed, e.g., in Wang, W. (1999), Instability, stabilization and formulation of liquid protein pharmaceuticals, Int J Pharm 185:129-188. Stability can be measured at a selected temperature for a selected time period. For rapid screening, for example, the formulation can be kept at 40 °C for 2 weeks to 1 month, at which time stability is measured. Where the formulation is to be stored at 2-8 °C, generally the formulation should be stable at 30 °C for at least 1 month, or 40 °C for at least a week, and/or stable at 2-8 °C for at least two years.

A protein “retains its physical stability” in a formulation if it shows minimal signs of changes to the secondary and/or tertiary structure (i.e., intrinsic structure), or aggregation, and/or precipitation and/or denaturation upon visual examination of color and/or clarity, or as measured by UV light scattering or by size exclusion chromatography, or other suitable methods. Physical instability of a protein, i.e., loss of physical stability, can be caused by oligomerization resulting in dimer and higher order aggregates, subvisible, and visible particle formation, and precipitation. The degree of physical degradation can be ascertained using varying techniques depending on the type of degradant of interest. Dimers and higher order soluble aggregates can be quantified using size exclusion chromatography, while subvisible particles may be quantified using light scattering, light obscuration or other suitable techniques.

A protein “retains its chemical stability” in a formulation, if the chemical stability at a given time is such that covalent bonds are not made or broken, resulting in changes to the primary structure of the protein component, e.g., antibody. Changes to the primary structure may result in modifications of the secondary and/or tertiary and/or quaternary structure of the protein and may result in formation of aggregates or reversal of aggregates already formed. Typical chemical modifications can include isomerization, deamidation, N-terminal cyclization, backbone hydrolysis, methionine oxidation, tryptophan oxidation, histidine oxidation, beta-elimination, disulfide formation, disulfide scrambling, disulfide cleavage, and other changes resulting in changes to the primary structure including D-amino acid formation. Chemical instability, i.e., loss of chemical stability, may be interrogated by a variety of techniques including ion-exchange chromatography, capillary isoelectric focusing, analysis of peptide digests and multiple types of mass spectrometric techniques. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by charge-based methods, such as, but not limited to, ion-exchange chromatography, capillary isoelectric focusing, or peptide mapping.

Loss of physical and/or chemical stability may result in changes to biological activity as either an increase or decrease of a biological activity of interest, depending on the modification and the protein being modified. A protein “retains its biological activity” in a buffered solution or formulation, if the biological activity of the protein at a given time is within about 30% of the biological activity exhibited at the time the formulation was prepared. Activity is considered decreased if the activity is less than 70% of its starting value. Biological assays may include both in vivo and in vitro based assays such as ligand binding, potency, cell proliferation or other surrogate measure of its biopharmaceutical activity.

Quantification of immunoglobulin protein (e.g., a single domain antibody or nanobody), is often useful or necessary in tracking protein. An antibody that specifically binds a domain of the nanobody or nanobodies of interest can therefore be useful for these purposes.

In some embodiments the antigen-binding protein of the invention is an antibody. The term “antibody”, or interchangeably “Ab,” is used in the broadest sense and includes fully assembled antibodies, monoclonal antibodies (including human, humanized or chimeric antibodies), monomeric, homodimeric, and heterodimeric antibodies, polyclonal antibodies, multi-specific antibodies (e.g., bispecific antibodies), single domain antibodies (sdAbs), and antibody fragments that can bind antigen (e.g., Fab, Fab′, F(ab′)₂, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. The term “antibody” encompasses genetically engineered and/or otherwise modified forms of immunoglobulins, such as intrabodies, peptibodies, chimeric antibodies, fully human antibodies, humanized antibodies, and heteroconjugate antibodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes have antibody-dependent cellular cytotoxicity (ADCC) activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies that are antigen binding proteins are highly specific binders, being directed against an individual antigenic site or epitope, in contrast to polyclonal antibody preparations that typically include different antibodies directed against different epitopes. Nonlimiting examples of monoclonal antibodies include murine, rabbit, rat, chicken, chimeric, humanized, or human antibodies, fully assembled antibodies, multispecific antibodies (including bispecific antibodies), antibody fragments that can bind an antigen (including, Fab, Fab′, F(ab)₂, Fv, single chain antibodies, diabodies), maxibodies, nanobodies, and recombinant peptides comprising CDRs of the foregoing as long as they exhibit the desired biological activity, or variants or derivatives thereof.

The modifier “monoclonal” indicates the character of the sdAb or antibody as being obtained from a substantially homogeneous population of sdAb or antibodies, and is not to be construed as requiring production of the sdAb or antibody by any particular method. For example, monoclonal antibodies may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

In a conventional “antibody” (i.e., homodimeric antibody), each tetramer is composed of two identical pairs of polypeptide chains, each pair having one “light” chain of about 220 amino acids (about 25 kDa) and one “heavy” chain of about 440 amino acids (about 50-70 kDa). The amino-terminal portion of each chain includes a “variable” (“V”) region of about 100 to 110 or more amino acids primarily responsible for antigen recognition. The carboxy-terminal portion of each chain defines a constant region primarily responsible for effector function. The variable region differs among different antibodies. The constant region is the same among different antibodies. Within the variable region of each heavy or light chain, there are three hypervariable subregions that help determine the antibody's specificity for antigen in the case of an antibody that is an antigen binding protein. The variable domain residues between the hypervariable regions are called the framework residues and generally are somewhat homologous among different antibodies. Immunoglobulins can be assigned to different classes depending on the amino acid sequence of the constant domain of their heavy chains. Human light chains are classified as kappa (κ) and lambda (λ) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)). An “antibody” also encompasses a recombinantly made antibody, and antibodies that are glycosylated or lacking glycosylation.

The term “light chain” or “immunoglobulin light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” or “immunoglobulin heavy chain” includes a full-length heavy chain and fragments thereof having sufficient variable region sequence to confer binding specificity. A full-length heavy chain includes a variable region domain, VH, and three constant region domains, C_{H1}, CH_{H2}, and CH_{H3}. The VH domain is at the amino-terminus of the polypeptide, and the C_{H} domains are at the carboxyl-terminus, with the C_{H3} being closest to the carboxy-terminus of the polypeptide. Heavy chains are classified as mu (μ), delta (δ), gamma (γ), alpha (α), and epsilon (ε), and define the antibody's isotype as IgM, IgD, IgG, IgA, and IgE, respectively. Heavy chains may be of any isotype, including IgG (including IgG1, IgG2, IgG3 and IgG4 subtypes), IgA (including IgA1 and IgA2 subtypes), IgM and IgE. Several of these may be further divided into subclasses or isotypes, e.g. IgG1, IgG2, IgG3, IgG4, IgA1 and IgA2. Different IgG isotypes may have different effector functions (mediated by the Fc region), such as antibody-dependent cellular cytotoxicity (ADCC) and complement-dependent cytotoxicity (CDC). In ADCC, the Fc region of an antibody binds to Fc receptors (Fc.gamma.Rs) on the surface of immune effector cells such as natural killers and macrophages, leading to the phagocytosis or lysis of the targeted cells. In CDC, the antibodies kill the targeted cells by triggering the complement cascade at the cell surface.

An “Fc region”, or used interchangeably herein, “Fc domain” or “immunoglobulin Fc domain”, contains two heavy chain fragments, which in a full antibody comprise the C_{H1} and CH₂ domains of the antibody. The two heavy chain fragments are held together by two or more disulfide bonds and by hydrophobic interactions of the C_{H3} domains.

The term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

For a detailed description of the structure and generation of antibodies, see Roth, D. B., and Craig, N. L., Cell, 94:411-414 (1998), herein incorporated by reference in its entirety. Briefly, the process for generating DNA encoding the heavy and light chain immunoglobulin sequences occurs primarily in developing B-cells. Prior to the rearranging and joining of various immunoglobulin gene segments, the V, D, J and constant (C) gene segments are found generally in relatively close proximity on a single chromosome. During B-cell-differentiation, one of each of the appropriate family members of the V, D, J (or only V and J in the case of light chain genes) gene segments are recombined to form functionally rearranged variable regions of the heavy and light immunoglobulin genes. This gene segment rearrangement process appears to be sequential. First, heavy chain D-to-J joints are made, followed by heavy chain V-to-DJ joints and light chain V-to-J joints. In addition to the rearrangement of V, D and J segments, further diversity is generated in the primary repertoire of immunoglobulin heavy and light chains by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and J_{H} segments and may extend over as many as 10 nucleotides. Furthermore, several nucleotides may be inserted between the D and J_{H} and between the V_{H} and D gene segments which are not encoded by genomic DNA. The addition of these nucleotides is known as N-region diversity. The net effect of such rearrangements in the variable region gene segments and the variable recombination which may occur during such joining is the production of a primary antibody repertoire.

The term “hypervariable” region refers to the amino acid residues of an antibody which are responsible for antigen-binding. The hypervariable region comprises amino acid residues from a complementarity determining region or CDR [i.e., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, th Ed. Public Health Service, National Institutes of Health, Bethesda, Md. (1991)]. Even a single CDR may recognize and bind antigen, although with a lower affinity than the entire antigen binding site containing all of the CDRs.

An alternative definition of residues from a hypervariable “loop” is described by Chothia et al., J. Mol. Biol. 196: 901-917 (1987) as residues 26-32 (L1), 50-52 (L2) and 91-96 (L3) in the light chain variable domain and 26-32 (H1), 53-55 (H2) and 96-101 (H3) in the heavy chain variable domain.

“Framework” or “FR” residues are those variable region residues other than the hypervariable region residues.

“Antibody fragments” comprise a portion of an intact full length antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “FE” fragment which contains the constant region. The Fab fragment contains all of the variable domain, as well as the constant domain of the light chain and the first constant domain (CH1) of the heavy chain. The Fc fragment displays carbohydrates and is responsible for many antibody effector functions (such as binding complement and cell receptors), that distinguish one class of antibody from another.

Pepsin treatment yields an F(ab′)₂ fragment that has two “Single-chain Fv” or “scFv” antibody fragments comprising the V_{H} and V_{L} domains of antibody, wherein these domains are present in a single polypeptide chain. Fab fragments differ from Fab′ fragments by the inclusion of a few additional residues at the carboxy terminus of the heavy chain CH1 domain including one or more cysteines from the antibody hinge region. Preferably, the Fv polypeptide further comprises a polypeptide linker between the VH and VL domains that enables the Fv to form the desired structure for antigen binding. For a review of scFv see Pluckthun in The Pharmacology of Monoclonal Antibodies, vol. 1 13, Rosenburg and Moore eds., Springer-Verlag, New York, pp. 269-315 (1994).

A “Fab fragment” is comprised of one light chain and the C_{H1} and variable regions of one heavy chain. The heavy chain of a Fab molecule cannot form a disulfide bond with another heavy chain molecule.

A “Fab′ fragment” contains one light chain and a portion of one heavy chain that contains the VH domain and the C_{H1} domain and also the region between the C_{H1} and C_{H2} domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H1} and C_{H2} domains, such that an interchain disulfide bond is formed between the two heavy chains. A F(ab′)₂ fragment thus is composed of two Fab′ fragments that are held together by a disulfide bond between the two heavy chains.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VL dimer. A single variable domain (or half of an Fv comprising only three CDRs specific for an antigen) has the ability to recognize and bind antigen, although at a lower affinity than the entire binding site.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference in their entireties.

“Single-chain Fv” or “scFv” antibody fragments comprise the V_{H} and V_{L} domains of antibody, wherein these domains are present in a single polypeptide chain, and optionally comprising a polypeptide linker between the V_{H} and V_{L} domains that enables the Fv to form the desired structure for antigen binding (Bird et al., Science 242:423-426, 1988, and Huston et al., Proc. Nati. Acad. Sci. USA 85:5879-5883, 1988). An “Fd” fragment consists of the V_{H} and C_{H1} domains.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VH regions of a bivalent domain antibody may target the same or different antigens.

The term “antigen binding protein” (ABP) includes antibodies or antibody fragments, as defined herein, that specifically bind a target ligand or antigen of interest.

In general, an antigen binding protein, e.g., an immunoglobulin protein, or an antibody or antibody fragment, “specifically binds” to a target ligand or antigen of interest when it has a significantly higher binding affinity for, and consequently is capable of distinguishing, that target ligand or antigen, compared to its affinity for other unrelated proteins, under similar binding assay conditions. There are two expressions called “equilibrium dissociation constant” (abbreviated herein “K_{D}” or “K_{d}”), which are commonly used to define the affinity of a protein for a ligand. The value of Kr is a kinetic term; it is the ratio of the off-rate (k_{back}) and on-rate (k_{forward}) constants. The formula for the value of Kr is the following:

K_{D}=k_{back}/k_{forwawd}=k_{off}/kₒₙ=(k_{d}/kₐ), where (I)

k_{back}=k_{off}=k_{d},i.e., the “dissociation rate constant,” and (II)

k_{forward}=kₒₙ=kₐ,i.e., the “association rate constant.” (III)

On the other hand, the value of K_{d} is calculated by the following formula:

K_{d}=([total binding sites]×[total ligand])/[PL]. (IV)

For an antigen-binding protein with a single binding site, K_{d} can be calculated from the concentrations of the antigen-binding protein (P), the ligand (L) and the P-L complex (PL), at equilibrium, with K_{d}=[P][L]/[PL].

The values of K_{D} and K_{d} are typically equivalent for a binding protein having a single binding site. Typically, an antigen binding protein is said to “specifically bind” its target antigen when the equilibrium dissociation constant (K_{d} or K_{D}) is 10⁻⁸ M or lower. The antigen binding protein specifically binds antigen with “high affinity” when the equilibrium dissociation constant is 10⁻⁹ M or lower, and with “very high affinity” when the K_{d} or K_{D} is 10⁻¹⁰ M or lower. A number of nanobodies are disclosed herein having different affinities to fentanyl, as well as to carfentanil. Differing sensitivities or affinities to a plurality or multiplicity of different opioids amongst nanobody species can be useful for distinguishing between each opioid species. For example, in a multiplexed assay, differing affinities to a single target (e.g., fentanyl and/or carfentanil) can also be useful. Of particular importance are uses in which a plurality of species of nanobodies are simultaneously deployed, in which differing affinities to the target can be helpful to ascertain analyte information (such as concentration) from a single preparation of sample, whereas an inconvenient dilution series may need to be prepared if only one species of nanobody were to be used. Other uses of relatively low-affinity nanobodies can include those in which relatively weak binding is desirable, for examples, when it is desirable to easily dissociate the nanobody-antigen pair in order to recycle the nanobodies. In addition, antigen-binding proteins (e.g., nanobodies) with relatively lower fentanyl affinity can have other desirable properties, such as but not limited to, a relatively higher affinity to other opioid species, or an increased stability to environmental conditions (e.g., temperature or pH).

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity; the GCG program package includes GAP (Devereux et al., 1984, Nucl. Acid Res. 12:387; Genetics Computer Group, University of Wisconsin, Madison, WI). The computer algorithm GAP is used to align the two polypeptides or two polynucleotides for which the percent sequence identity is to be determined. The sequences are aligned for optimal matching of their respective amino acid or nucleotide (the “matched span”, as determined by the algorithm). A gap opening penalty (which is calculated as 3.times. the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following: Algorithm: Needleman et al., 1970, J. Mol. Biol. 48:443-453;
Comparison matrix: BLOSUM 62 from Henikoff et al., 1992, supra;
Gap Penalty: 12 (but with no penalty for end gaps);
Gap Length Penalty: 4;
Threshold of Similarity: 0.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The term “modification” when used in connection with proteins of interest, include, but are not limited to, one or more amino acid changes (including substitutions, insertions or deletions); chemical modifications; covalent modification by conjugation to therapeutic or diagnostic agents; labeling (e.g., with radionuclides or various enzymes); covalent polymer attachment such as PEGylation (derivatization with polyethylene glycol) and insertion or substitution by chemical synthesis of non-natural amino acids. By methods known to the skilled artisan, proteins, can be “engineered” or modified for improved target affinity, selectivity, stability, and/or manufacturability before the coding sequence of the “engineered” protein is included in the expression cassette.

The term “derivative,” when used in connection with proteins of interest, refers to proteins that are covalently modified by conjugation to therapeutic or diagnostic agents, labeling (e.g., with radionuclides or various enzymes), covalent polymer attachment such as PEGylation (derivatization with polyethylene glycol) and insertion or substitution of natural or non-natural amino acids.

Conservatively modified forms of the antigen-binding proteins disclosed herein are also contemplated as being embodiments of the present invention. A “conservative amino acid substitution” may involve a substitution of a native amino acid residue with a non-native or non-canonical residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al, Acta Physiol. Scand. SuppL, 643:55-67 (1998); Sasaki et al, 1998, Adv. Biophys. 35: 1-24 (1998), which discuss alanine scanning mutagenesis).

Desired amino acid substitutions (whether conservative or non-conservative) can be determined by those skilled in the art at the time such substitutions are desired. For example, amino acid substitutions can be used to identify important residues of the peptide sequence, or to increase or decrease the affinity of the peptide or vehicle-conjugated peptide molecules described herein.

Naturally occurring residues may be divided into classes based on common side chain properties:
1) hydrophobic: norleucine (Nle), Met, Ala, Val, Leu, Ile;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Trp, Tyr, Phe.

Conservative amino acid substitutions may involve exchange of a member of one of these classes with another member of the same class. Conservative amino acid substitutions may encompass non-naturally occurring amino acid residues (e.g., norleucine (Nle)), which are typically incorporated by chemical peptide synthesis rather than by synthesis in biological systems. These include peptidomimetics and other reversed or inverted forms of amino acid moieties.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al, 1982, J. Mol. Biol. 157: 105-131). It is known that certain amino acids may be substituted for other amino acids having a similar hydropathic index or score and still retain a similar biological activity. In making conservative amino acid substitutions based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the conservative amino acid substitutions of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included. One may also identify epitopes from primary amino acid sequences on the basis of hydrophilicity. These regions are also referred to as “epitopic core regions.”

Examples of conservative amino acid substitutions include the substitution of one non-polar (hydrophobic) amino acid residue such as isoleucine, valine, leucine, norleucine (Nle), alanine, or methionine for another, the substitution of one polar (hydrophilic) amino acid residue for another such as between arginine and lysine, between glutamine and asparagine, between glycine and serine, the substitution of one basic amino acid residue such as lysine, arginine or histidine for another, or the substitution of one acidic residue, such as aspartic acid or glutamic acid for another. The phrase “conservative amino acid substitution” also includes the use of a chemically derivatized residue in place of a non-derivatized residue, provided that such polypeptide displays the requisite bioactivity. Other exemplary amino acid substitutions that can be useful in accordance with the present invention are set forth in Table A below.

TABLE A
Some Useful Conservative Amino Acid Substitutions.
Conventional three-letter abbreviations are shown.
“Nle” means norleucine.

| Original | Exemplary |
|---|---|
| Residue | Substitutions |
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala, Phe, Nle |
| Leu | Ile, Val, Met, Ala, Phe, Nle |
| Lys | Arg, Gln, Asn |
| Met | Leu, Phe, Ile |
| Phe | Leu, Val, Ile, Ala, Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, Ala, Nle |

### Cloning DNA

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the polypeptide of interest, e.g., antibody sequences.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

Sequencing of DNA is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of genes and cDNAs, one of skill will readily be able to determine sequence identity, depending on the region sequenced. One source of gene sequence information is the National Center for Biotechnology Information, National Library of Medicine, National Institutes of Health, Bethesda, MD.

Embodiments of the present method of enhancing in vitro recombinant expression of a protein of interest by a mammalian host cell or the method of manufacturing a protein drug substance of interest can involve so-called “Next-generation” sequencing, as a preferred method for confirming the presence of all engineered DNA constructs prior to the transfection step(s). (See, e.g., Buermans, H. P. J., & den Dunnen, J. T., Next generation sequencing technology: Advances and applications, Biochimica et Biophysica Acta—Molecular Basis of Disease 1842(10): 1932-1941 (2014)). Sanger will provide an indication of the species present but not as individual designs. Sequencing will validate that the absence of any species was not due to their absence as a DNA construct. There is a possibility that some engineered designs will not be expressed and secreted at high enough levels to survive all processing steps and be detected by mass spectrometry. This may result because certain engineered antibody variant designs are unstable, but such variants will not likely be viable as therapeutics anyway. This can be viewed as part of the screening process, however, since typically it is desirable to find antibody variant candidates that do express well for manufacturing purposes.

Chemical synthesis of parts or the whole of a coding region containing codons reflecting desired protein changes can be cloned into an expression vector by either restriction digest and ligation of 5′ and 3′ ends of fragments or the entire open reading frame (ORF), containing nucleotide overhangs that are generated by restriction enzyme digestion and which are compatible to the destination vector. The fragments or inserts are typically ligated into the destination vector using a T4 ligase or other common enzyme. Other useful methods are similar to the above except that the cut site for the restriction enzyme is at location different from the recognition sequence. Alternatively, isothermal assembly (i.e., “Gibson Assembly”) can be employed, in which nucleotide overhangs are generated during synthesis of fragments or ORFs; digestion by exonucleases is employed. Alternatively, nucleotide overhangs can be ligated ex vivo by a ligase or polymerase or in vivo by intracellular processes.

Alternatively, homologous recombination can be employed, similar to isothermal assembly, except exonuclease activity of T4 DNA ligase can used on both insert and vector and ligation can be performed in vivo.

Another useful cloning method is the so-called “TOPO” method, in which a complete insert containing a 3′ adenosine overhang (generated by Taq polymerase) is present, and Topoisomerase I ligates the insert into a TOPO vector.

Another useful cloning method is degenerate or error-prone PCR exploiting degenerate primers and/or a thermally stable low-fidelity polymerase caused by the polymerase within certain reaction conditions. Fragments or inserts are then cloned into an expression vector.

The above are merely examples of known cloning techniques, and the skilled practitioner knows how to employ any other suitable cloning techniques.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components can include one or more of the following: a signal sequence (that may, for example, direct secretion of the expressed protein by the recombinant host cells); an origin of replication, one or more selection marker or reporter genes (that may, for example, encode a fluorescent protein, such as a green fluorescent protein (GFP), an enhanced green fluorescent protein (EGFP), a red-shifted green fluorescent protein (rs-GFP), a yellow fluorescent protein (YFP), a red fluorescent protein (RFP), a cyan fluorescent protein (e.g., CyOFP1), mini Singlet Oxygen Generator (miniSOG), a luminescent protein (e.g., luciferase), or the like, or may confer antibiotic or other drug resistance, or complement auxotrophic deficiencies of the host cells or supply critical nutrients not available in the medium, e.g., dihydrofolate reductase or glutamine synthetase selection markers), an enhancer element, a promoter, and a transcription termination sequence, all of which are well known in the art.

### Protein Expression and Cell Culture

The inventive method for manufacturing a purified protein of interest (e.g., but not limited to, a protein drug substance) involves culturing protein-secreting mammalian cells. Such cultured mammalian cells are typically made by recombinant DNA technology involving transient or stable transfection, e.g., the pooled plasmid constructs (expression vectors) from the cloning step can be transfected into a plurality of host cells (e.g., mammalian, e.g., HEK 293 or CHO cell, or insect cells, or microbial host cells, e.g., bacterial cells, yeast cells, or algal or microalgal cells) for expression using a cationic lipid, polyethylenimine, Lipofectamine™, or ExpiFectamine™, or electroporation. The skilled practitioner is aware of numerous suitable means for transfecting to achieve expression of recombinant antibodies. Alternatively, methods for stable genomic integration of expressions cassettes encoding the protein of interest can be employed to make a production cell line of protein-secreting mammalian cells. (See, e.g., Zhang, Crispr-Cas Systems and Methods for Altering Expression Of Gene Products, WO2014093661 A2; Frendewey et al., Methods and Compositions for the Targeted Modification of a Genome, U.S. Pat. No. 9,228,208 B2; Church et al., Multiplex Automated Genome Engineering, WO2008052101A2, U.S. Pat. No. 8,153,432 B2; Bradley et al., Methods Cells and Organisms, US2015/0079680 A1; Begemann et al., Compositions and Methods for Modifying Genomes, WO2017141173A2; Gill et al., Nucleic acid-guided nucleases, U.S. Pat. No. 9,982,279 B1; Minshull et al., Enhanced nucleic acid constructs for eukaryotic gene expression, U.S. Pat. No. 9,428,767B2, U.S. Pat. No. 9,580,697B2, U.S. Pat. No. 9,574,209B2; Minshull et al., DNA Vectors, Transposons And Transposases For Eukaryotic Genome Modification, U.S. Ser. No. 10/041,077B2; McGrew et al., Hybrid Promoter and Uses Thereof, U.S. Ser. No. 11/028,410B2; McGrew et al., Expression from Transposon-Based Vectors and Uses, U.S. Ser. No. 11/098,310B2; McGrew et al., Inducible Expression From Transposon-Based Vectors and Uses, US 2019/0185881A1).

Optionally, the transfectant or transformant cells will be provided with a recombinant expression cassette for a selectable marker, for example, but not limited to, one or more of the following: glutamine synthase, dihydrofolate reductase, puromycin-N acetyl transferase, blasticidin-S deaminase, hygromycin phosphotransferase, aminoglycoside phosphotransferase, nourseothricin N-acetyl transferase, or a protein that binds to zeocin.

The protein of interest is typically obtained by culturing the transfected or transformed host cells under physiological conditions allowing the cells to express recombinant proteins. Most conveniently, the expressed recombinant proteins are directly secreted into the extracellular culture medium (by employing appropriate secretory-directing signal peptides) and are harvested therefrom; otherwise additional steps will be needed to isolate the expressed antibodies from a cell extract.

Useful secretory signal peptide (SP) sequences are known in the art, and these can be added, adjacent or distal, to any of the sequences shown in Table 1, herein, for the purpose of facilitating secretion of the inventive antigen-binding protein. An example of a useful SP sequence is the IGKV1-39*01 SP signal peptide:

MDMRVPAQLLGLLLLWLRGARC (SEQ ID NO: 58). Other examples of useful SP sequences include:
MEAPAQLLFLLLLWLPDTTG (SEQ ID NO: 59),
MEWTWRVLFLVAAATGAHS (SEQ ID NO: 60),
METPAQLLFLLLLWLPDTTG (SEQ ID NO: 61),
MKHLWFFLLLVAAPRWVLS (SEQ ID NO: 62),
MEWSWVFLFFLSVTTGVHS (SEQ ID NO: 63), but any other suitable signal peptide sequence may be employed within the scope of the invention.

The desired scale of the recombinant expression will be dependent on the type of expression system and the desired quantity of protein production. Some expression systems such as ExpiCHO™ usually produce higher yields as compared to some earlier HEK293 technologies. A smaller scale ExpiCHO™ might then suffice as compared to an HEK293 system. Efficiency of transfection can also be a consideration in choosing an appropriate expression system. Electroporation can be a suitable method given its effectiveness, relative low cost and the fact that high-throughput during this step is not critical. Additionally, the ratio of immunoglobulin light chain to heavy chain can be varied during the co-transfection to improve expression of certain variants. The product yield for a given variant has to be sufficient to survive numerous handling steps and produce a signal high enough to be detected by the chosen fluorescence detector.

In general, the transfected or transformed host cells are typically cultured by any conventional type of culture, such as batch, fed-batch, intensified fed-batch, or continuous. Suitable continuous cultures included repeated batch, chemostat, turbidostat or perfusion culture with product and cell retention or solely cell retention. Bioreactors for protein production, which can be reusable or single-use bioreactors, typically can contain a volume of liquid culture medium of about 50 L to about 4000 L (e.g., 50 L, 60 L, 75 L, 100 L, 250 L, 500 L, 650 L, 750 L, 1000 L, 1250 L, 1500 L, 1750 L, 2000 L, 2250 L, 2500 L, 2750 L, 3000 L, 3250 L, 3500 L, 3750 L, or 4000 L), as desired.

The host cells used to produce the protein of interest or “POI” (e.g., non-glycosylated or glycosylated proteins) in the invention can be cultured in a variety of media suitable for the type of host cell chosen. Commercially available media such as Ham's F10 (Sigma-Aldrich), Minimal Essential Medium (MEM) (Sigma-Aldrich), RPMI-1640 (Sigma-Aldrich), and Dulbecco's Modified Eagle's Medium ((DMEM), Sigma-Aldrich) can be suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. patent Re. No. 30,985 can be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source, such that the physiological conditions of the cell in, or on, the medium promote expression of the protein of interest by the host cell; any other necessary supplements may also be included at appropriate concentrations that would be known to those skilled in the art.

In some embodiments, the host cell, in culture, is a microbial cell or microorganism (used interchangeably herein), e.g., a bacterium, a cyanobacterium, a fungus, a microalga, or an alga. The microbial cell can be a bacterium, such as, but not limited to, Escherichia coli, Bacillus subtilis, Salmonella sp., Aliivibrio fischeri, Pseudomonas fluorescens, Bacillus sp., Cupriavidus metallidurans, Deinococcus radiodurans, and Staphylococcus aureus. In some embodiments, the microbial cell is a fungus, such as, but not limited to, Saccharomyces cerevisiae, Pichia pastoris, Kluyveromyces lactis, Hansenula polymorpha, and Yarrowia lipolytica, and Trichosporon cutaneum. In some embodiments, the microbial host cell is Synechocystis sp. As an example, synthetic constructs can be transformed into both standard E. coli MG1655 (ATCC 700926) and optimized E. coli LABEC01 cells for expression of proteins of interest. The E. coli MG1655 lab strain can be grown in lysogeny broth (LB) medium and has been adapted and has been adapted for growth in M9 minimal medium. (See, e.g., Hasty et al., WO2016/133830A1, US2018/149633A1).

The ordinarily skilled artisan is familiar with useful culture conditions, such as temperature (for mammalian cells, typically, but not necessarily, about 37°±1 °C), pH (typically, but not necessarily, the cell culture medium is maintained within the range of about pH 6.5-7.5), oxygenation, and the like. By “culturing at” or “maintaining at” a predetermined culture condition, is meant that the process control systems are set at a particular value for that condition, in other words the intended volume, target temperature, pH, oxygenation level, or the like, maintained at predetermined set points for each parameter, within a narrow range (i.e., “narrow deadband”) most optimal for the cell line and protein product of interest. Clearly, there will be small variations of the temperature, pH, or other culture condition over time, and from location to location through the culture vessel (i.e., the bioreactor). (See, also, e.g., Oguchi et al., pH Condition in temperature shift cultivation enhances cell longevity and specific hMab productivity in CHO culture, Cytotechnology. 52(3):199-207 (2006); Al-Fageeh et al., The cold-shock response in cultured mammalian cells: Harnessing the response for the improvement of recombinant protein production, Biotechnol. Bioeng. 93:829-835 (2006); Marchant, R. J. et al., Metabolic rates, growth phase, and mRNA levels influence cell-specific antibody production levels from in vitro cultured mammalian cells at sub-physiological temperatures, Mol. Biotechnol. 39:69-77 (2008)).

Digital control units and sensory monitors are available commercially or can be constructed by the skilled artisan for use with cell culture bioreactors. Alternative digital control units (DCU) control and monitor the cell culture process are available commercially, made by companies such as B. Braun, New Brunswick, Sartorius, or Thermo Fisher Scientific. Other on-line or off-line analyses can include off-gas measurements by mass spectrometry, in-depth determination of media composition (amino acids, vitamins, trace minerals) and expanded examination of cellular metabolites in addition to CO₂ and lactic acid.

Examples of epitope tags include the flu HA tag polypeptide and its antibody 12CA5 [Field et al, Mol. Cell. Biol. 8: 2159-2165 (1988)]; the c-myc tag and the 8F9, 3C7, 6E10, G4, B7 and 9E10 antibodies thereto [Evan et al, Mol. Cell. Biol. 5(12): 3610-3616 (1985)]; and the Herpes Simplex virus glycoprotein D (gD) tag and its antibody [Paborsky et al, Protein Engineering 3(6): 547-553 (1990)]. Other exemplary tags are a poly-histidine sequence, generally around six histidine residues, that permits isolation of a compound so labeled using nickel chelation. Other labels and tags, such as the FLAG® tag (Eastman Kodak, Rochester, NY) are well known and routinely used in the art.

Thus an epitope tag peptide, can be used to facilitate purification and/or detection of the antigen-binding protein of the invention. In some cases, the tagging peptide is detectable by itself (e.g. fluorescent tags such as GFP) while in other cases the tagging peptide is detectable because it specifically binds a detectable molecule (in turn, the detectable molecule can be directly detectable, e.g. fluorescent, or it may be detected by specific binding to it of a detectable molecule, i.e. a scaffold of molecules may be required for detection). If used for purification and/or indirect detection, such a peptide is usually designed (or found) to have a high affinity to a readily, or even commercially, available antibody molecule. Such peptides are often derived from a species unrelated to the species where the polypeptides is intended to be used to avoid any cross reaction, especially during detection. The molecule binding the tagging peptide may be selected for its detectability and/or for ease of immobilization and/or recovery in purification processes. Common tagging peptide include HA-tag (a short peptide from human influenza hemagglutinin), Flag-tag, His-tag or hexa-histidine (comprising at least 6 histidine residues) and the Strep-tag (comprising eight amino acids and which is readily bound by commercially available Strep-tactin (an engineered streptavidin) and antibodies). In particular embodiments, the V_{HH}-comprising polypeptide of the invention comprises a Strep-tag fused C-terminally to the V_{HH}, particularly intercalated between the V_{HH} and effector peptide. The Strep-tag system for one-step purification and high-affinity detection or capturing of proteins can be useful in immunoaffinity purification of the inventive proteins. (See, e.g., Schmidt et al., “The Strep-tag system for one-step purification and high-affinity detection or capturing of proteins,” Nature Protocols volume 2, pages 1528-1535 (2007)).

Some particular, non-limiting, embodiments of amino acid substitution variants of the inventive immunoglobulins, including antibodies and antibody fragments are exemplified below.

Any cysteine residue not involved in maintaining the proper conformation of the immunoglobulin also may be substituted, generally with serine, to improve the oxidative stability of the molecule and prevent aberrant crosslinking. Conversely, cysteine bond(s) may be added to the immunoglobulin to improve its stability (particularly where the immunoglobulin is an antibody fragment such as an Fv fragment).

In certain instances, immunoglobulin variants are prepared with the intent to modify those amino acid residues which are directly involved in epitope binding in a starting sequence. In other embodiments, modification of residues which are not directly involved in epitope binding or residues not involved in epitope binding in any way, is desirable, for purposes discussed herein. Mutagenesis within any of the CDR regions and/or framework regions is contemplated.

In order to determine which antigen binding protein amino acid residues are important for epitope recognition and binding, alanine scanning mutagenesis can be performed to produce substitution variants. See, for example, Cunningham et al, Science, 244: 1081-1085 (1989), the disclosure of which is incorporated herein by reference in its entirety. In this method, individual amino acid residues are replaced one-at-a-time with an alanine residue and the resulting antibody is screened for its ability to bind its specific epitope relative to the unmodified polypeptide. Modified antigen binding proteins with reduced binding capacity are sequenced to determine which residue was changed, indicating its significance in binding or biological properties.

Substitution variants of antigen binding proteins can be prepared by affinity maturation wherein random amino acid changes are introduced into the parent polypeptide sequence. See, for example, Ouwehand et al, Vox Sang 74 (Suppl 2):223-232, 1998; Rader et al, Proc. Natl. Acad. Sci. USA 95:8910-8915, 1998; DaU'Acqua et al, Curr. Opin. Struct. Biol. 8:443-450, 1998, the disclosures of which are incorporated herein by reference in their entireties. Affinity maturation involves preparing and screening the antigen binding proteins, or variants thereof and selecting from the resulting variants those that have modified biological properties, such as increased binding affinity relative to the parent antigen binding protein. A convenient way for generating substitutional variants is affinity maturation using phage display. Briefly, several hypervariable region sites are mutated to generate all possible amino substitutions at each site. The variants thus generated are expressed in a monovalent fashion on the surface of filamentous phage particles as fusions to the gene III product of M1 3 packaged within each particle. The phage-displayed variants are then screened for their biological activity (e.g., binding affinity). See e.g., WO 92/01047, WO 93/112366, WO 95/15388 and WO 93/19172.

Current antibody affinity maturation methods belong to two mutagenesis categories: stochastic and nonstochastic. Error prone PCR, mutator bacterial strains (Low et al, J. Mol. Biol. 260, 359-68, 1996), and saturation mutagenesis (Nishimiya et al, J. Biol. Chem. 275: 12813-20, 2000; Chowdhury, P. S. Methods Mol. Biol. 178, 269-85, 2002) are typical examples of stochastic mutagenesis methods (Rajpal et al, Proc Natl Acad Sci USA. 102:8466-71, 2005). Nonstochastic techniques often use alanine-scanning or site-directed mutagenesis to generate limited collections of specific muteins. Some methods are described in further detail below.

Affinity maturation via panning methods—Affinity maturation of recombinant antibodies is commonly performed through several rounds of panning of candidate antibodies in the presence of decreasing amounts of antigen. Decreasing the amount of antigen per round selects the antibodies with the highest affinity to the antigen thereby yielding antibodies of high affinity from a large pool of starting material. Affinity maturation via panning is well known in the art and is described, for example, in Huls et al. (Cancer Immunol Immunother. 50: 163-71, 2001). Methods of affinity maturation using phage display technologies are described elsewhere herein and known in the art (see e.g., Daugherty et al, Proc Natl Acad Sci USA. 97:2029-34, 2000).

So-called “look-through mutagenesis” (LTM) provides a method for rapidly mapping an antibody-binding site. (Rajpal et al., “A general method for greatly improving the affinity of antibodies by using combinatorial libraries,” Proc Natl Acad Sci USA 102(24):8466-71 (2005)). For LTM, nine amino acids, representative of the major side-chain chemistries provided by the 20 natural amino acids, are selected to dissect the functional side-chain contributions to binding at every position in all six CDRs of an antibody. LTM generates a positional series of single mutations within a CDR where each “wild type” residue is systematically substituted by one of nine selected amino acids. Mutated CDRs are combined to generate combinatorial single-chain variable fragment (scFv) libraries of increasing complexity and size without becoming prohibitive to the quantitative display of all muteins. After positive selection, clones with improved binding are sequenced, and beneficial mutations are mapped. [00356] Error-prone PCR— Error-prone PCR involves the randomization of nucleic acids between different selection rounds. The randomization occurs at a low rate by the intrinsic error rate of the polymerase used but can be enhanced by error-prone PCR (Zaccolo et al, J. Mol. Biol. 285:775-783, 1999) using a polymerase having a high intrinsic error rate during transcription (Hawkins et al., J Mol Biol. 226:889-96, 1992). After the mutation cycles, clones with improved affinity for the antigen are selected using routine methods in the art.

Techniques utilizing gene shuffling and directed evolution may also be used to prepare and screen antigen binding proteins, or variants thereof, for desired activity. For example, Jermutus et al, Proc Natl Acad Sci USA, 98(1):75-80 (2001) showed that tailored in vitro selection strategies based on ribosome display were combined with in vitro diversification by DNA shuffling to evolve either the off-rate or thermodynamic stability of scFvs; Fermer et al., Tumour Biol. 2004 January-April; 25(1-2):7-13 reported that use of phage display in combination with DNA shuffling raised affinity by almost three orders of magnitude. Dougherty et al., Proc Natl Acad Sci USA. 2000 Feb. 29; 97(5):2029-2034 reported that (i) functional clones occur at an unexpectedly high frequency in hypermutated libraries, (ii) gain-of-function mutants are well represented in such libraries, and (iii) the majority of the scFv mutations leading to higher affinity correspond to residues distant from the binding site.

Alternatively, or in addition, it may be beneficial to analyze a crystal structure of the antigen-antibody complex to identify contact points between the antibody and antigen, or to use computer software to model such contact points. Such contact residues and neighboring residues are candidates for substitution according to the techniques elaborated herein. Once such variants are generated, they are subjected to screening as described herein and antibodies with superior properties in one or more relevant assays may be selected for further development.

Immunoglobulins with modified carbohydrate [00360] Immunoglobulin variants can also be produced that have a modified glycosylation pattern relative to the parent polypeptide, for example, adding or deleting one or more of the carbohydrate moieties bound to the immunoglobulin, and/or adding or deleting one or more glycosylation sites in the immunoglobulin.

Glycosylation of polypeptides, including antibodies is typically either N-linked or O-linked. N-linked refers to the attachment of the carbohydrate moiety to the side chain of an asparagine residue. The tripeptide sequences asparagine-X-serine and asparagine —X-threonine, where X is any amino acid except proline, are the recognition sequences for enzymatic attachment of the carbohydrate moiety to the asparagine side chain. The presence of either of these tripeptide sequences in a polypeptide creates a potential glycosylation site. Thus, N-linked glycosylation sites may be added to an immunoglobulin by altering the amino acid sequence such that it contains one or more of these tripeptide sequences. O-linked glycosylation refers to the attachment of one of the sugars N-aceylgalactosamine, galactose, or xylose to a hydroxyamino acid, most commonly serine or threonine, although 5-hydroxyproline or 5-hydroxy lysine may also be used. O-linked glycosylation sites may be added to an immunoglobulin by inserting or substituting one or more serine or threonine residues to the sequence of the original immunoglobulin or antibody.

### Altered Effector Function

Embodiments of the antigen-binding proteins of the present invention can be fused to Fc-containing domains. In such embodiments, cysteine residue(s) may be removed or introduced in the Fc region of an antibody or Fc-containing polypeptide, thereby eliminating or increasing interchain disulfide bond formation in this region. A homodimeric immunoglobulin thus generated may have improved internalization capability and/or increased complement-mediated cell killing and antibody-dependent cellular cytotoxicity (ADCC). See Caron et al, J. Exp Med. 176: 1191-1195 (1992) and Shopes, B. J. Immunol. 148: 2918-2922 (1992). Homodimeric immunoglobulins or homodimeric antibodies (i.e., antibodies having two copies of a single LC monomer species and a single HC monomer species) may also be prepared using heterobifunctional cross-linkers as described in Wolff et al., Cancer Research 53: 2560-2565 (1993). Alternatively, an immunoglobulin can be engineered which has dual Fe regions and may thereby have enhanced complement lysis and ADCC capabilities. See Stevenson et al., Anti-CancerDrug Design 3: 219-230 (1989).

It is also contemplated that one or more of the N-terminal 20 amino acid residues (e.g., a signal sequence) of the heavy and/or light chain are removed, or amino acid residues are deleted from the C-terminal, for example, amino acid sequences from which one, two, three, four or five amino acid residues are lacking from the N-terminal or C-terminal, or from both.

Modifications to increase serum half-life also may desirable, for example, by incorporation of or addition of a salvage receptor binding epitope (e.g., by mutation of the appropriate region or by incorporating the epitope into a peptide tag that is then fused to the immunoglobulin at either end or in the middle, e.g., by DNA or peptide synthesis) (see, e.g., WO96/32478) or adding molecules such as PEG or other water soluble polymers, including polysaccharide polymers.

The salvage receptor binding epitope preferably constitutes a region wherein any one or more amino acid residues from one or two loops of a Fc domain are transferred to an analogous position of the immunoglobulin or fragment. Even more preferably, three or more residues from one or two loops of the Fc domain are transferred. Still more preferred, the epitope is taken from the C_{H}2 domain of the Fc region (e.g., of an IgG) and transferred to the C_{H}1, C_{H}3, or VH region, or more than one such region, of the immunoglobulin or antibody. Alternatively, the epitope is taken from the C_{H2} domain of the Fc region and transferred to the C_{L} region or V_{L} region, or both, of the immunoglobulin fragment. See also International applications WO 97/34631 and WO 96/32478 which describe Fc variants and their interaction with the salvage receptor.

Other sites and amino acid residue(s) of the constant region have been identified that are responsible for complement dependent cytotoxicity (CDC), such as the C1q binding site, and/or the antibody-dependent cellular cytotoxicity (ADCC) [see, e.g., Molec. Immunol. 29 (5): 633-9 (1992); Shields et al, J. Biol. Chem., 276(9):6591-6604 (2001); Lazar et al, Proc. Nat'l. Acad. Sci. 103(11): 4005 (2006) which describe the effect of mutations at specific positions, each of which is incorporated by reference herein in its entirety]. Mutation of residues within Fc receptor binding sites can result in altered (i.e. increased or decreased) effector function, such as altered affinity for Fc receptors, altered ADCC or CDC activity, or altered half-life. As described above, potential mutations include insertion, deletion or substitution of one or more residues, including substitution with alanine, a conservative substitution, a non-conservative substitution, or replacement with a corresponding amino acid residue at the same position from a different subclass (e.g. replacing an IgG1 residue with a corresponding IgG2 residue at that position).

The invention also encompasses production of immunoglobulin molecules, including antibodies and antibody fragments, with altered carbohydrate structure resulting in altered effector activity, including antibody molecules with absent or reduced fucosylation that exhibit improved ADCC activity. A variety of ways are known in the art to accomplish this. For example, ADCC effector activity is mediated by binding of the antibody molecule to the FcγRIII receptor, which has been shown to be dependent on the carbohydrate structure of the N-linked glycosylation at the Asn-297 of the C_{H2} domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (Yamane-Ohnuki et al, Biotechnol Bioeng. 2004 Sep. 5; 87(5):614-22). Similar effects can be accomplished through decreasing the activity of this or other enzymes in the fucosylation pathway, e.g., through siRNA or antisense RNA treatment, engineering cell lines to knockout the enzyme(s), or culturing with selective glycosylation inhibitors (Rothman et al., Mol Immunol. 1989 December; 26(12): 1113-23). Some host cell strains, e.g. Lecl3 or rat hybridoma YB2/0 cell line naturally produce antibodies with lower fucosylation levels. Shields et al, J Biol Chem. 2002 Jul. 26; 277(30):26733-40; Shinkawa et al, J Biol Chem. 2003 Jan. 31; 278(5):3466-73. An increase in the level of bisected carbohydrate, e.g. through recombinantly producing antibody in cells that overexpress GnTIII enzyme, has also been determined to increase ADCC activity. Umana et al., Nat Biotechnol. 1999 February; 17(2): 176-80. It has been predicted that the absence of only one of the two fucose residues may be sufficient to increase ADCC activity. (Ferrara et al., J Biol Chem. 2005 Dec. 5).

### Variable Domains of Antigen-Binding Proteins, e.g., Nanobodies

Exemplary amino acid sequences of single variable domain (V_{HH}) and encompassed CDR sequences of the inventive antigen-binding proteins are provided in Table 1 (see, below). So for example, the isolated recombinant antigen-binding protein can include a V_{HH} domain comprising an amino acid sequence selected from the group consisting of SEQ ID NO: 1, SEQ ID NO: 5, SEQ ID NO: 9, SEQ ID NO: 10, SEQ ID NO: 11, SEQ ID NO: 15, SEQ ID NO: 16, SEQ ID NO: 20, SEQ ID NO: 21, SEQ ID NO: 22, SEQ ID NO: 23, SEQ ID NO: 24, SEQ ID NO: 28, SEQ ID NO: 29, SEQ ID NO: 33, SEQ ID NO: 37, SEQ ID NO: 38, SEQ ID NO: 39, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 52, SEQ ID NO: 53, and SEQ ID NO: 54; or can comprise any one of the foregoing sequences from which one, two, three, four or five amino acid residues are lacking from the N-terminal or C-terminal, or from both.

Ordinarily, amino acid sequence variants of the immunoglobulin sequences disclosed herein will have an amino acid sequence having at least 60% amino acid sequence identity with the original or reference immunoglobulin domain (e.g., V_{HH}) amino acid sequence. Amino acid variant sequences of a V_{HH} domain sequence of the invention can also have at least 65%, or at least 70%, or at least 75% or at least 80% amino acid sequence identity, more preferably at least 85% sequence identity, even more preferably at least 90%) sequence identity, and most preferably at least 95% sequence identity, including for example, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity. Identity or homology with respect to this sequence is defined herein as the percentage of amino acid residues in the sequence that are identical with the original or reference sequence, after aligning the sequences and candidate introducing gaps, if necessary, to achieve the maximum percent sequence identity, and not considering any conservative amino acid substitutions as part of the sequence identity. None of N-terminal, C-terminal, or internal extensions, deletions, or insertions into the immunoglobulin or antibody sequence shall be construed as affecting sequence identity or homology.

Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an immunoglobulin with an N-terminal methionyl residue or the immunoglobulin (including antibody or antibody fragment) fused to an epitope tag or a salvage receptor binding epitope, which can be useful in detection and/or purification of the inventive proteins of interest. Other insertional variants of the immunoglobulin or antibody molecule include the fusion to a polypeptide which increases the serum half-life of the immunoglobulin, e.g. at the N-terminus or C-terminus. Typically, V_{HH} domains include 2 cysteine amino acid residues (hereafter cysteines) that can form an intramolecular disulphide bond; they do not contain an odd number of cysteines that could form intermolecular disulphide bonds, and, generally do not have the capacity to form homodimers. However, fusion proteins of V_{HH} or other multimeric V_{HH}s can be produced. (See, Prehaud et al. Nanobodies Suitable for Neuron Regeneration Therapy, US2021/040187A1). In some embodiments, it may be advantageous to design a V_{HH} or a V_{HH}-comprising polypeptide of the invention so that is has dimerization capacity. In particular, disulphide bond-forming cysteines may be included, especially in the N-terminal region of the V_{HH} or V_{HH}-comprising polypeptide, e.g. in the sequence of the neuron cell-targeting peptide mentioned above. This does not result in decreased expression in bacteria used for their production and results in enhanced activity of the construct. V_{HH} or V_{HH}-comprising polypeptides forming disulphide bonds are readily expressed as dimers in the periplasm of bacteria and the recovery of the construct is easy. It is also possible that the formation of dimers increases the half-life of the V_{HH} or V_{HH}-comprising polypeptide of the invention. In particular embodiments, therefore, the V_{HH} or V_{HH}-comprising polypeptide of the invention has dimerization, especially homodimerization capacity, preferably through the formation of disulphide bonds and/or through domains in the N-terminal region of the V_{HH} or V_{HH}-comprising polypeptide of the invention.

Purified antigen-binding proteins of the present invention (e.g., sdAbs or nanobodies) can be employed in most commonly accepted immunological assay systems, including but not limited to, lateral flow test strips, enzyme-linked immunosorbent assay (ELISA) systems, radioimmunoassay (RIA) systems, latex agglutination (LA) assay systems or any other agglutination reaction-based assay system, surface plasmon resonance (SPR), or electrochemical methods, including capacitive or piezoelectric biosensors within which nanobodies are immobilized on a surface and changes in electrical properties (e.g., capacitance, impedance, or voltage frequency) change when the antigen-binding proteins binds to its target antigen. Such assay system embodiments can also employ host cells displaying the antigen-binding proteins on their surfaces.

Host cells that display on their surfaces the antigen-binding proteins of the present invention can be employed in other assay system embodiments. For example, non-continuous type assay system devices include a microtiter plate-based assay system, in which sample is added to wells and agglutination is measured, or a single-use tube-based assay system in which sample is added to a tube (e.g., a 1.5-mL microcentrifuge tube) and agglutination is measured. In both of these sorts of assay system embodiments, agglutination can be measured using any suitable measurement or detection modalities, e.g., measurement of transmitted or absorbed light, scattered light, fluorescence, flow cytometry, microscopy, or electrical impedance, or by-eye by visualized pellet formation, and correlating the change in the measured parameter (e.g., turbidity, fluorescence, electrical impedance, mean particle size, or the like), with the level of agglutination. In a continuous type assay, such as the inventive continuous agglutination assay method and continuous agglutination assay system, described herein, the change in the measured parameter is correlated over a time course with the level of agglutination. This “continuous time course” is at least about 1 to 45 minutes, or about 5-45 minutes, or about 10-30 minutes; e.g., an approximately 30-minute period typically provides a useful time course for the inventive continuous agglutination assay method and continuous agglutination assay system. If a light-based parameter (e.g., transmission, scattering, or absorbance of light) is elected as convenient to be the measured parameter, it can be measured usefully in the visible or infrared spectra, such as but not limited to, wavelengths of 860 nm, 800 nm, 700 nm, 650 nm, 600 nm, or the like.

TABLE 1
Single Variable Domain (VHH) and encompassed
CDR sequences

| SEQ | VHH | |
|---|---|---|
| ID | Designation/ | |
| NO: | Description | Amino Acid Sequence |
| 1 | A01 | QVQLVESGGGLAQAGGSLRLSCTGSGRTSNVNIVGWFR |
| | | QPPGKEREFVAAIRWRDLRTWYPYYADSVKGRFALST |
| | | NNAANTVFLQMNSLKPEDTA VYYCACAARARPRTGE |
| | | WDTSVTGARDYDYWGQGTQVTVSS |
| 2 | CDR1 | RTSNVNIVG |
| 3 | CDR2 | AIRWRDLRTWY |
| 4 | CDR3 | CAARARPRTGEWDTSVTGARDYDY |
| 5 | A03 | EVOLVESGGGLVQVGGSLRLSCTASGRAYESPIAGWFR |
| | | QAPGKEREFVATLLWSSGSTDYADPVKGRFTISKGTTD |
| | | NMVYLQMNNLKADDTAIYYCAAATVFRGRLASRLPTS |
| | | YDYWGQGTQVTVSS |
| 6 | CDR1 | RAYESPIAG |
| 7 | CDR2 | TLLWSSGSTDY |
| 8 | CDR3 | AATVFRGRLASRLPTSYDY |
| 9 | A05 | QVQLVESVGGLVQVGGSLRLSCTASGRAYESPIAGWFR |
| | | QAPGKEREFVATLLWSSGSTDYADPVKGRFTISKGTTD |
| | | NMVYLQMNNLKADDTAIYYCAAATVFRGRLASRLPTS |
| | | YDYWGQGTQVTVSS |
| 6 | CDR1 | RAYESPIAG |
| 7 | CDR2 | TLLWSSGSTDY |
| 8 | CDR3 | AATVFRGRLASRLPTSYDY |
| 10 | A06 | EVOLVESGGGLAQAGGSLRLSCTGSGRTSNVNIVGWFR |
| | | QPPGKEREFVAAIRWRDLRTWYPYYADSVKGRFALST |
| | | NNAANTVFLQMNSLKPEDTAVYYCACAARARPRTGE |
| | | WDTSVTGARDYDYWGQGTQVTVSS |
| 2 | CDR1 | RTSNVNIVG |
| 3 | CDR2 | AIRWRDLRTWY |
| 4 | CDR3 | CAARARPRTGEWDTSVTGARDYDY |
| 11 | A07 | QVQLVESVGGLVQVGGSLRLSCTASGRAYSSPIVGWFR |
| | | QAPGKEREFVATLFWHSGSTDYGDPVKGRFTVSRATA |
| | | DNMVYLOMNSLKAEDTAIYYCAAATIFRGRLGYRLPT |
| | | AYDYWGQGTQVTVSS |
| 12 | CDR1 | RAYSSPIVG |
| 13 | CDR2 | TLFWHSGSTDY |
| 14 | CDR3 | AATIFRGRLGYRLPTAYDY |
| 15 | A09 | QVQLVESGGGLVQVGGSLRLSCTASGRA YESPIAGWFR |
| | | QAPGKEREFVATLLWSSGSTDYADPVKGRFTISKGTTD |
| | | NMVYLQMNNLKADDTAIYYCAAATVFRGRLASRLPTS |
| | | YDYWGQGTQVTVSS |
| 6 | CDR1 | RAYESPIAG |
| 7 | CDR2 | TLLWSSGSTDY |
| 8 | CDR3 | AATVFRGRLASRLPTSYDY |
| 16 | A11 | QVQLVESVGGWVQAGGSLRLSCTASGSIFSINDMAWY |
| | | RQATGKQRERVAVLVRNYTHYLDSVKGRFTISRDNAN |
| | | AKIALNLQMNSLKPEDIAVYFCNANTESRAGPEYWGQG |
| | | TQVTVSS |
| 17 | CDR1 | SIFSINDMA |
| 18 | CDR2 | VLVRNYTHY |
| 19 | CDR3 | ANTESRAGPEY |
| 20 | B01 | QVQLVESGGGLAQAGGSLRLSCTGSGRTSNVNIVGWFR |
| | | QPPGKEREFVAAIRWRDLRTWYPYYADSVKGRFALST |
| | | NNAANTVFLQMNSLKPEDTAVYYCACAARARPRTGE |
| | | WDTSVTGARDYDYWGQGTLVTVSS |
| 2 | CDR1 | RTSNVNIVG |
| 3 | CDR2 | AIRWRDLRTWY |
| 4 | CDR3 | CAARARPRTGEWDTSVTGARDYDY |
| 21 | B04 | QVQLVESGGGLVQVGGSLRLSCTASGRAYSSPIVGWFR |
| | | QAPGKEREFVATLFWHSGSTDYGDPVKGRFTVSRATA |
| | | DNMVYLQMNSLKAEDTAIYYCAAATIFRGRLGYRLPT |
| | | AYDYWGQGTQVTVSS |
| 12 | CDR1 | RAYSSPIVG |
| 13 | CDR2 | TLFWHSGSTDY |
| 14 | CDR3 | AATIFRGRLGYRLPTAYDY |
| 22 | B08 | QVQLVESGGGLAQAGGSLRLSCTGSGRTSNVNIVGWFR |
| | | QPPGKEREFVAAIRWRDLRTWYPYYADSVKGRFALST |
| | | NNAANTVFLQMNSLKPVDTAVYYCACAARARPRTGE |
| | | WDTSVTGARDYDYWGQGTQVTVSS |
| 2 | CDR1 | RTSNVNIVG |
| 3 | CDR2 | AIRWRDLRTWY |
| 4 | CDR3 | CAARARPRTGEWDTSVTGARDYDY |
| 23 | B12 | EVOLVESGGGLVQVGGSLRLSCTASGRAYSSPIVGWFR |
| | | QAPGKEREFVATLFWHSGSTDYGDPVKGRFTVSRATA |
| | | DNMVYLQMNSLKAEDTAIYYCAAATIFRGRLGYRLPT |
| | | AYDYWGQGTLVTVSS |
| 12 | CDR1 | RAYSSPIVG |
| 13 | CDR2 | TLFWHSGSTDY |
| 14 | CDR3 | AATIFRGRLGYRLPTAYDY |
| 24 | C01 | QVQLVESGGGLVQAGGSLSLSCSASGRAFRSYVVGWF |
| | | RQAPGKERYFVASINWGSLRTTYGDGVKGRFTISRDAA |
| | | KNTVNLQMNSLKPEDTAVYYCAAMSGSSYATTPRSSA |
| | | DEYDYWGQGTLVTVSS |
| 25 | CDR1 | RAFRSYVVG |
| 26 | CDR2 | SINWGSLRTTY |
| 27 | CDR3 | AMSGSSY ATTPRSSADEYDY |
| 28 | C08 | QVQLVESGGGLVQVGGSLRLSCTASGRA YESPIAGWFR |
| | | QAPGKEREFVATLLWSSGSTDYADPVKGRFTISKGATD |
| | | NMVYLQMNNLKADDTAIYYCAAATVFRGRLASRLPTS |
| | | YDYWGQGTLVTVSS |
| 6 | CDR1 | RAYESPIAG |
| 7 | CDR2 | TLLWSSGSTDY |
| 8 | CDR3 | AATVFRGRLASRLPTSYDY |
| 29 | C10 | EVOLVESGGGLVQAGGSLRLSCAASGRTFSNYVMGWF |
| | | RLAPGKEREFVAAIHWNIGKTIYADSAKGRFTISRDNAK |
| | | NTVYLQMNSLKPEDTALYYCVATWRGASIDMPTTANE |
| | | YRYRGQGTLVTVSS |
| 30 | CDR1 | RTFSNYVMG |
| 31 | CDR2 | AIHWNIGKTIY |
| 32 | CDR3 | ATWRGASIDMPTTANEYRY |
| 33 | C12 | QVQLVESGGGLVQPGGSLRLSCAASGFTLGDYDIGWFR |
| | | QAPGKERVGVSCATRREGNTWYADSVKGRFVISRDNA |
| | | KNTVYLQMRSLKFEDTAVYYCAVPCWGRGRYWGQGT |
| | | QVTVSS |
| 34 | CDR1 | FTLGDYDIG |
| 35 | CDR2 | CATRREGNTWY |
| 36 | CDR3 | VPCWGRGRY |
| 37 | D02 | QVQLVESGGGLVQAGGSLSLTCSASGRAFRSYVVGWF |
| | | RQAPGKERYFVASINWGSLRTTYGDGVKGRFTISRDVA |
| | | KNTVNLQMNSLKPEDTAVYYCAAMSGSSYATTPRSSA |
| | | DEYDYWGQGTQVTVSS |
| 25 | CDR1 | RAFRSYVVG |
| 26 | CDR2 | SINWGSLRTTY |
| 27 | CDR3 | AMSGSSY ATTPRSSADEYDY |
| 38 | E01 | EVOLVESGGGLAQAGGSLRLSCAGSGRTSNVNIVGWFR |
| | | QPPGKEREFVAAIRWRDLRTWYPYYADSVKGRFALST |
| | | NNAANTVFLQMNSLKPEDTAVYYCACAARARPRTGE |
| | | WDTSVTGARDYDYWGQGTQVTVSS |
| 2 | CDR1 | RTSNVNIVG |
| 3 | CDR2 | AIRWRDLRTWY |
| 4 | CDR3 | CAARARPRTGEWDTSVTGARDYDY |
| 39 | E04 | QVQLVESGGGLVQPGGSLRLSCVASGNTYGIDVMGWY |
| | | RQAPGEQRELVAIITSGGSTNYADSVKGRFTISAEYAKN |
| | | TVYLQMNSLTSEDTAVYTCHAHESRWSMRGKTREYDY |
| | | WGQGTQVTVSS |
| 40 | CDR1 | NTYGIDVMG |
| 41 | CDR2 | IITSGGSTNY |
| 42 | CDR3 | AHESRWSMRGKTREYDY |
| 43 | E05 | QVQLVESGGGLAQAGGSLRLSCTGSGRTSNVNIVGWFR |
| | | QPPGKEREFVAAIRWRDLRTWYPYYADSVKGRFALST |
| | | NNAANTVFLQMNSLKPEDTAVYYCAARQGSNWYLHP |
| | | DYWGQGTLVTVSS |
| 2 | CDR1 | RTSNVNIVG |
| 3 | CDR2 | AIRWRDLRTWY |
| 44 | CDR3 | ARQGSNWYLHPDY |
| 45 | E06 | EVOLVESGGGLARAGGSLRLSCTGSGRTSNVNIVGWFR |
| | | RPPGKEREFVAAIRWRDLRTWYPYYADSVKGRFALST |
| | | NNAANTVFLOMNSLKPEDTAVYYCACAARARPRTGE |
| | | WDTSVTGARDYDYWGQGTQVTVSS |
| 2 | CDR1 | RTSNVNIVG |
| 3 | CDR2 | AIRWRDLRTWY |
| 4 | CDR3 | CAARARPRTGEWDTSVTGARDYDY |
| 46 | E07 | EVOLVESGGRLVQAGGSLRLSCTGSGRTSNVNIVGWFR |
| | | QPPGKEREFVAAIRWRDLRTWYPYYADSVKGRFALST |
| | | NNAANTVFLQMNSLKPEDTAVYYCACAARARPRTGE |
| | | WDTSVTGARDYDYWGQGTQVTVSS |
| 2 | CDR1 | RTSNVNIVG |
| 3 | CDR2 | AIRWRDLRTWY |
| 4 | CDR3 | CAARARPRTGEWDTSVTGARDYDY |
| 47 | E08 | QVQLVESVGGLVQAGGSLRLSCTGSGRTSNVNIVGWFR |
| | | QPPGKEREFVAAIRWRDLRTWYPYYADSVKGRFALST |
| | | NNAANTVFLQMNSLKPEDTAVYYCACAARARPRTGE |
| | | WDTSVTGARDYDYWGQGTQVTVSS |
| 2 | CDR1 | RTSNVNIVG |
| 3 | CDR2 | AIRWRDLRTWY |
| 4 | CDR3 | CAARARPRTGEWDTSVTGARDYDY |
| 48 | E09 | EVOLVESGGGLAQAGGSLRLSCTGSGRTSNVNIVGWFR |
| | | QPPGKEREFVAAIRWRDLRTWYPYYADSVKGRFALST |
| | | NNAANTVFLQMNSLKPEDTAVYYCACAARARPRTGE |
| | | WDTSVTGARDYDYWGQGTLVTVSS |
| 2 | CDR1 | RTSNVNIVG |
| 3 | CDR2 | AIRWRDLRTWY |
| 4 | CDR3 | CAARARPRTGEWDTSVTGARDYDY |
| 49 | E12 | EVOLVESGGGLVQVGGSLRLSCTASGRAYSSPIVGWFR |
| | | QAPGKEREFVATLFWHSGSTDYGDPVKGRFTVSRATA |
| | | DNMVYLQMNSLKAEDTAIYYCAAATIFRGRLGYRLPT |
| | | AYDYWGQGTQVTVSS |
| 12 | CDR1 | RAYSSPIVG |
| 13 | CDR2 | TLFWHSGSTDY |
| 14 | CDR3 | AATIFRGRLGYRLPTAYDY |
| 50 | F03 | EVOLVESGGGLVQAGGSLSLSCSASGRAFRSYVVGWFR |
| | | QAPGKERYFVASINWGSLRTTYGDGVKGRFTISRDVAK |
| | | NTVNLQMNSLKPEDTAVYYCAAMSGSSYATTPRSSAN |
| | | EYDYWGQGTLVTVSS |
| 25 | CDR1 | RAFRSYVVG |
| 26 | CDR2 | SINWGSLRTTY |
| 51 | CDR3 | AMSGSSYATTPRSSANEYDY |
| 52 | G09 | EVOLVESGGGLVQVGGSLRLSCTASGRAYSSPIVGWFR |
| | | QAPGKEREFVATLFWHSGSTDYGDPVKGRFTVSRATA |
| | | DNMVYLQMNSLKAEDTAIYYCAAATIFRGRLGYRLPT |
| | | AYDYWGRGTQVTVSS |
| 12 | CDR1 | RAYSSPIVG |
| 13 | CDR2 | TLFWHSGSTDY |
| 14 | CDR3 | AATIFRGRLGYRLPTAYDY |
| 53 | G11 | QVQLVESGGGLVQAGGSLRLSCTASGRAYSSPIVGWFR |
| | | QAPGKEREFVATLFWHSGSTDYGDPVKGRFTVSRATA |
| | | DNMVYLQMNSLKAEDTAIYYCAAATIFRGRLGYRLPT |
| | | AYDYWGQGTQVTVSS |
| 12 | CDR1 | RAYSSPIVG |
| 13 | CDR2 | TLFWHSGSTDY |
| 14 | CDR3 | AATIFRGRLGYRLPTAYDY |
| 54 | H02 | QVQLVESGGGLVQPGGSLTLSCVVSGIRFSVHTVGWYR |
| | | QGPGSQREFVASAGPHSRTDYAHFVRGRFTISRDEAKN |
| | | TAYLQMANLQPEDTAVYYCARDGTSPIPEFDCWGQGT |
| | | QVTVSS |
| 55 | CDR1 | IRFSVHTVG |
| 56 | CDR2 | SAGPHSRTDY |
| 57 | CDR3 | RDGTSPIPEFDC |

### Pumps

The pumps of the inventive system or device can be peristaltic or positive displacement. Pump heads can be single-use and product-contacting pump heads that can be replaced from run to run or traditional, non-product-contacting pump heads, such as but not limited to, typical peristaltic pumps, which are reusable. For example, in some embodiments the pump (for high flow rate operation) is a Quattroflow 1200SU HT, single-use quaternary diaphragm pump with a flow rate range of about 6 to 1200 L/hour; a pump (for low flow rate operation) is a Quattroflow™ 150SU pump motor retrofitted with Quattroflow™ 30SU pump head with flow rate range of about 0.06 to about 30 L/hour.

### Valves

The automated valves (when present) of the inventive system or device can be of any suitable type to regulate fluid flow through the connecting lines. In some embodiments the automated valves are robotically regulated mechanical valves, pneumatic valves, hydraulic valves, diaphragm valves, ball valve, gate valves, needle valves, bellows valves, or globe valves. If a source of compressed air is available, pneumatic valves are particularly useful. A pinch valve type, which has a block that drops down onto the tubing to pinch the tubing of a connecting line shut is a useful embodiment (e.g., pinch valves manufactured by Aquasyn LLC or Acro Associates, a wholly-owned subsidiary of Bimba Manufacturing Corporation). A variety of pinch valves are useful, including, but not limited to, overmolded tubing assemblies to reduce dead volume in the tubing. There are other useful options that include multiplexing valves together, e.g., in 3-way or 4-way configurations. The skilled person knows how to select the optimal valves for a given embodiment, in view of the desired level of simplicity, cost effectiveness, and the nature of the tubing assembly chosen, with a view to minimizing valve footprint and dead volumes.

### Controllers.

In some embodiments the addition to the reaction vessel for mixing, of a fluid aqueous suspension of host cells that display on their surfaces a plurality of recombinant antigen-binding proteins, and/or a fluid aqueous sample, and/or an agglutinating agent comprising the analyte of interest is accomplished manually, e.g., with appropriately sized pipettes, micropipettes, or other liquid handling apparati. Between runs, vessels and tubing of the connecting lines can be cleaned or replaced manually, as well. However, in other embodiments, the microcontroller of the inventive system or device of the present invention can be configured and programmable to automatically control any, or all, of these operations (e.g., sampling, mixing of reaction mixture reagent(s), system cleaning, etc.), switching the flow or path of aqueous fluid(s) through the system or device independently, based on pre-programmed instructions, set-points, and/or measurement signals transmitted by optional flowmeters and/or other analytical sensors monitoring, e.g., absorbance, UV, fluorescence, conductivity, temperature, and/or pH. In some embodiments, the microcontroller is configured and programmable to automatically switch the flow of aqueous fluid from one vessel to a different vessel, or to an outlet, in response to a predetermined set-point or measurement signal received from a sensor(s), and the microcontroller is further configured and programmable to issue instruction signals to other automated components of the system. In some embodiments of the inventive system or device, microcontroller can be configured and programmable to analyze the data from the analytical sensors, or, optionally, the data analysis can be by a separate programmable computer.

By way of further illustration, the following numbered embodiments are encompassed by the present invention:

Embodiment 1: An isolated recombinant antigen-binding protein that specifically binds fentanyl or carfentanil, comprising a V_{HH} domain comprising a set of three complementarity determining regions (CDR): CDR1, CDR2, and CDR3, wherein each CDR comprises an amino acid sequence, wherein the set of three CDRs is selected from the group consisting of:

or wherein one or more of the CDR amino acid sequences of the set of three CDR amino acid sequences comprises a conservative amino acid substitution of one or two, preferably one, amino acid residues; and wherein the antigen binding protein does not specifically bind norfentanyl; and, optionally, wherein the isolated recombinant antigen-binding protein specifically binds fentanyl or carfentanil with an equilibrium dissociation constant (K_{d} or K_{D}) of ≤10⁻⁸ M, more preferably with a K_{d} or K_{D} of ≤5×10⁻⁹ M, and even more preferably with a K_{d} or K_{D} of ≤5×10⁻¹⁰ M, or even ≤10⁻¹ M, as measured, e.g., by ELISA or surface plasmon resonance.

Embodiment 2: The isolated recombinant antigen-binding protein of Embodiment 1, comprising a V_{HH} domain comprising an amino acid sequence selected from the group consisting of SEQ ID NO: 1, SEQ ID NO: 5, SEQ ID NO: 9, SEQ ID NO: 10, SEQ ID NO: 11, SEQ ID NO: 15, SEQ ID NO: 16, SEQ ID NO: 20, SEQ ID NO: 21, SEQ ID NO: 22, SEQ ID NO: 23, SEQ ID NO: 24, SEQ ID NO: 28, SEQ ID NO: 29, SEQ ID NO: 33, SEQ ID NO: 37, SEQ ID NO: 38, SEQ ID NO: 39, SEQ ID NO: 43, SEQ ID NO: 45, SEQ ID NO: 46, SEQ ID NO: 47, SEQ ID NO: 48, SEQ ID NO: 49, SEQ ID NO: 50, SEQ ID NO: 52, SEQ ID NO: 53, and SEQ ID NO: 54; or comprising any one of the foregoing sequences from which one, two, three, four or five amino acid residues are lacking from the N-terminal or C-terminal, or from both.

Embodiment 3: An isolated nucleic acid, comprising a nucleotide sequence encoding the recombinant antigen-binding protein according to any of Embodiments 1-2.

Embodiment 4: An expression vector, comprising the isolated nucleic acid of Embodiment 3.

Embodiment 5: A host cell, in culture, comprising the expression vector of Embodiment 4.

Embodiment 6: The host cell, in culture, of Embodiment 5, wherein the host cell is a microbial cell.

Embodiment 7: The host cell, in culture, of Embodiment 6, wherein the microbial cell is a bacterium, a cyanobacterium, a fungus, a microalga, or an alga.

Embodiment 8: The host cell, in culture, according to any of Embodiments 5-7, wherein the host cell is selected from the group consisting of Escherichia coli, Bacillus subtilis, Salmonella sp., Aliivibrio fischeri, Pseudomonas fluorescens, Bacillus sp., Cupriavidus metallidurans, Deinococcus radiodurans, and Staphylococcus aureus.

Embodiment 9: The host cell, in culture, according to any of Embodiments 5-7, wherein the host cell is selected from the group consisting of Saccharomyces cerevisiae and Trichosporon cutaneum.

Embodiment 10: The host cell, in culture, of Embodiment 5, wherein the host cell is a mammalian cell, an insect cell, or a plant cell.

Embodiment 11: The host cell, in culture, of Embodiment 10, wherein the mammalian cell is derived from a Chinese Hamster Ovary (CHO) cell.

Embodiment 12: The host cell, in culture, according to any of Embodiments 10-11, selected from the group consisting of a CHO-K1 cell, a DXB11 cell, and a DG44 cell.

Embodiment 13: The isolated recombinant antigen-binding protein according to any of Embodiments 1-2, being expressed on the surface of the host cell, in culture, according to any of Embodiments 5-12.

Embodiment 14: A device for detecting the presence of fentanyl and/or carfentanil, comprising the isolated recombinant antigen-binding protein according to any of Embodiments 1-2 and 13.

Embodiment 15: A device for detecting the presence of fentanyl and/or carfentanil, comprising the host cell according to any of Embodiments 5-12.

Embodiment 16: The device according to any of Embodiments 14-15, comprising a lateral flow immunodetection test strip, or an ELISA system or array, a radioimmunoassay (RIA) system, or an agglutination reaction-based assay system (such as but not limited to latex agglutination).

Embodiment 17: The device according to any of Embodiments 14-15, comprising a piezoelectric detector or a surface plasmon resonance sensor.

Embodiment 18: A pharmaceutical composition, comprising the antigen-binding protein according to any of Embodiments 1-2, and a pharmaceutically acceptable excipient.

Embodiment 19: A method of treating a patient addicted to opiates, comprising administering a therapeutically effective dose of the pharmaceutical composition of Embodiment 18.

Embodiment 20: A formulation comprising the pharmaceutical composition of Embodiment 18, for use in treating opiate addiction.

Embodiment 21: A method of producing an antigen-binding protein that specifically binds to fentanyl and/or carfentanil, comprising:
(a) culturing the host cell according to any of Embodiments 5-12, in an aqueous medium under physiological conditions permitting expression of the antigen-binding protein; and
(b) recovering the antigen-binding protein from the medium.

Embodiment 22: A continuous agglutination assay method for detecting an analyte of interest, comprising:
(a) mixing in a reaction vessel, a fluid aqueous suspension of host cells that display on their surfaces a plurality of recombinant antigen-binding proteins that specifically bind an analyte of interest, with a fluid aqueous sample, in a reaction mixture with an agglutinating agent comprising the analyte of interest, or an analyte conjugate, under conditions of temperature and pH that permit binding of the analyte or the analyte conjugate, by the antigen-binding protein and agglutination of the cells, in the presence of the analyte of interest;
(b) measuring a parameter in a preselected portion of the reaction mixture over a continuous time course;
(c) correlating the change in the measured parameter over the time course with the level of agglutination; and
(d) normalizing the level of agglutination relative to maximum and minimum control levels of agglutination to obtain a normalized value;
wherein the normalized value reflects the concentration of the analyte of interest in the aqueous sample over the continuous time course.

Embodiment 23: The continuous agglutination assay method according to Embodiment 22, wherein the step of measuring the level of agglutination in the reaction mixture is over a continuous time course of at least about 1 to 45 minutes.

Embodiment 24: The continuous agglutination assay method according to any of Embodiments 22-23, wherein the step of measuring the level of agglutination in the reaction mixture is through a fixed distance in the preselected portion of the reaction mixture in the reaction vessel.

Embodiment 25: The continuous agglutination assay method according to any of Embodiments 22-24, wherein the step of measuring the level of agglutination in the reaction mixture comprises measuring the turbidity of the reaction mixture.

Embodiment 26: The continuous agglutination assay method according to any of Embodiments 22-25, wherein the step of measuring the level of agglutination in the reaction mixture comprises detecting the transmission, scattering, or absorbance of light.

Embodiment 27: The continuous agglutination assay method according to any of Embodiments 22-26, wherein the preselected portion of the reaction mixture is in the upper half- to upper third-portion.

Embodiment 28: The continuous agglutination assay method according to any of Embodiments 22-27, further comprising adding a series of known concentrations of the analyte, or an analyte conjugate, to the reaction mixture to compete with the agglutinating agent, during a series of one or more further continuous time courses.

Embodiment 29: The continuous agglutination assay method according to any of Embodiments 22-28, wherein the host cells are fixed prior to use in the method.

Embodiment 30: The continuous agglutination assay method according to any of Embodiments 22-29, wherein the analyte of interest is fentanyl or carfentanil, and wherein the agglutinating agent comprises fentanyl, carfentanil, and/or a conjugate of either.

Embodiment 31: The continuous agglutination assay method according to any of Embodiments 22-30, wherein the plurality of recombinant antigen-binding proteins comprises one or more antigen-binding proteins according to any of Embodiments 1-2.

Embodiment 32: An automated system for continuous agglutination assay, comprising:
(a) a first vessel configured to receive and to contain, a fluid aqueous suspension comprising host cells that display on their surfaces a plurality of recombinant antigen-binding proteins that specifically bind an analyte of interest, and, wherein the fluid aqueous suspension optionally comprises an antibiotic or bacteriostatic compound, a coagulant and/or an agglutination-enhancing additive;
(b) an optional second vessel configured to receive and to contain a fluid aqueous sample to be analyzed;
(c) a reaction vessel fluidly connected to the first vessel and to the optional second vessel by connecting lines, wherein the reaction vessel is configured to automatically receive via the connecting lines, a predetermined volume of the fluid aqueous suspension from the first vessel and a predetermined volume of the fluid aqueous sample, optionally from the second vessel, or directly from a sample inlet, and to contain a reaction mixture comprising the host cells and the aqueous sample;
(d) a sensor configured to measure a parameter correlatable to the level of agglutination in the reaction mixture in the reaction vessel, in response to a measurement signal, and to transmit the thusly acquired measurement data;
(e) a waste outlet, or optionally a waste vessel, fluidly connected to the reaction vessel by a connecting line;
(f) a plurality of fluid propellers each configured to impel fluid flow through the connecting lines in response to a signal, wherein at least one fluid propeller is upstream from the reaction vessel, and at least one fluid head is downstream from the reaction vessel;
(g) a microcontroller configured and programmable to signal the plurality of fluid propellers to initiate fluid flow, impel fluid flow, or cease fluid flow, through the connecting lines:
   (iii) from the first vessel, and from the optional second vessel or the sample inlet, to the reaction vessel;
   (iv) from the reaction vessel to the waste outlet, or optionally to the waste vessel; and
      wherein the microcontroller, or optionally, a separate microcontroller, is further configured and programmable to transmit the measurement signal to the sensor, and to collect the measurement data from the sensor.

Embodiment 33: The automated system for continuous agglutination assay of Embodiment 32, comprising the optional second vessel, configured to receive and to contain the fluid aqueous sample to be analyzed.

Embodiment 34: The automated system for continuous agglutination assay according to any of Embodiments 32-33, wherein the analyte of interest is fentanyl or carfentanil, and the plurality of recombinant antigen-binding proteins comprises one or more antigen-binding proteins according to any of Embodiments 1-2.

Embodiment 35: The automated system for continuous agglutination assay according to any of Embodiments 32-34, wherein the reaction vessel, the first vessel, and/or the optional second vessel, further comprise a stirrer.

Embodiment 36: The automated system for continuous agglutination assay according to any of Embodiments 32-35, wherein the plurality of fluid propellers comprise one or more pump heads.

Embodiment 37: The automated system for continuous agglutination assay according to any of Embodiments 32-36, wherein the system is airtight, and the plurality of fluid propellers comprises one or more gas regulator(s) for regulating the pressure of an inert gas in the headspace of the first vessel, of the optional second vessel, and of the reaction vessel.

The following working examples are illustrative and not to be construed in any way as limiting the scope of the invention.

### EXAMPLES

### Example 1. Isolation of Single Domain Antibodies (sdAbs) Specific to Fentanyl

Immunizations. Conjugates of fentanyl to bovine serum albumin (BSA) and keyhole limpet hemocyanin (KLH) were prepared by AAT Bioquest (Sunnyvale, CA). A single llama was immunized with the fentanyl-KLH conjugate over a 14-week period. The primary immunization consisted of 0.5 mg of fentanyl-KLH administered in complete Freund's adjuvant. Booster injections of 0.5 mg of fentanyl-KLH in incomplete Freund's adjuvant were administered at 2, 4, 8, and 12 weeks. Blood samples (10 mL) were collected from the llama with the primary immunization and at week 6 to monitor total IgG titer. Two 500-mL production bleeds were performed, one at week 10 and the other at week 14.

Phage-display library. A phage-display library was created from the V_{HH} sequences obtained in the two production bleeds. Peripheral blood mononuclear cells (PBMCs) were isolated from the production bleeds within 4 hours of drawing. RNA was isolated from the PBMCs, cDNA was transcribed from the isolated RNA, and the V_{HH} sequences were inserted into phagemid DNA for expression in E. coli.

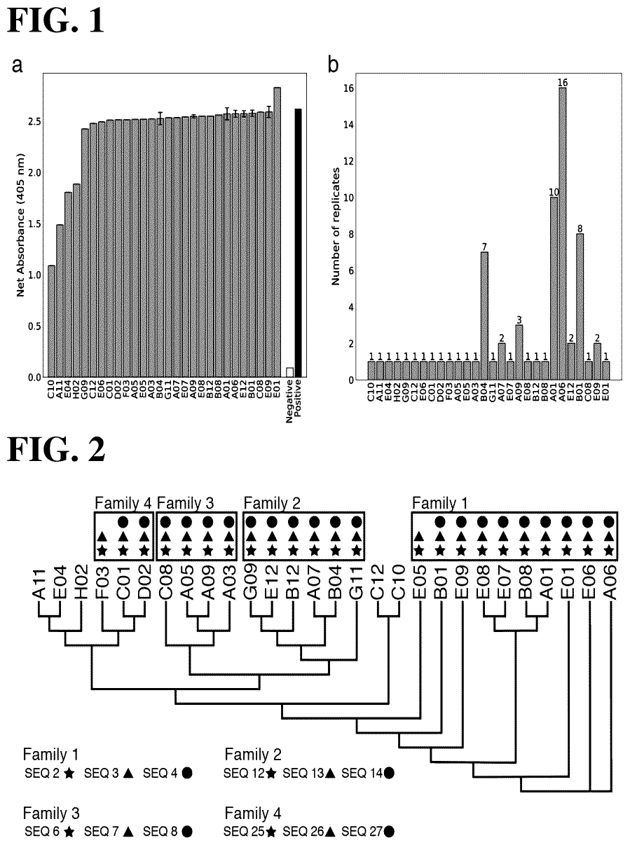

Three rounds of library panning were performed with fentanyl-BSA immobilized on high-bind plastic ELISA plates. After panning, the enriched phagemid pool was transformed into E. coli cells, from which 94 random colonies were selected for further screening. Histidine-tagged V_{HH} domains were recovered from the periplasmic fraction of E. coli by osmotic shock, and evaluated by two parallel ELISA assays: one against fentanyl-BSA conjugate and the other against unconjugated BSA to test for non-specific binding (FIG. 1). Sequencing of these 94 colonies uncovered 28 unique sequences. The 28 sequences were aligned with Clustal Omega software, with agglomerative clustering showing the relationship between each sequence (FIG. 2). (Sievers, Fabian et al., “Fast, scalable generation of high-quality protein multiple sequence alignments using Clustal Omega,” Molecular Systems Biology, 7. 539. doi: accession:10.1038/msb.2011.75 (2011)).

Of the 28 unique sequences, 23 sdAbs fell into one of four distinct families, of which each member shared sequences for at least two of the three complementarity determining regions (CDR). This clustering was used to select a representative subset of V_{HH} sequences to more closely investigate.

### Example 2. Surface Display of V_{HH} Domains in E. coli Towards Fentanyl Detection

Six V_{HH} sequences: A06 (SEQ ID NO: 10), A09 (SEQ ID NO: 15), B04 (SEQ ID NO: 21), CO₁ (SEQ ID NO: 24), C12 (SEQ ID NO: 33), and E01 (SEQ ID NO: 38), were cloned into an Intimin-based outer membrane expression vector in the EcM1 strain of E. coli, as described in Salema et al. (2013). (Salema, V. et al., “Selection of Single Domain Antibodies from Immune Libraries Displayed on the Surface of E. coli Cells with Two (3-Domains of Opposite Topologies,” PLoS ONE, 8(9):1-18 (2013). doi.org/10.1371/journal.pone.0075126).

This expression system enables the extracellular display of each V_{HH} linked to an outer membrane anchored β-barrel domain and allows for the V_{HH}s to be evaluated without purification. The function of the six V_{HH} sequences was first validated by an agglutination assay in which E. coli displaying a V_{HH} domain were incubated with a multivalent antigen, leading to extensive E. coli-antigen linking and the formation of large complexes that can be detected by eye. (See, Kylilis, N. et al., “Whole-cell biosensor with tunable limit of detection enables low-cost agglutination assays for medical diagnostic applications,” ACS Sensors, 4(2):370-378 (2019). doi.org/10.1021/acssensors.8b01163).

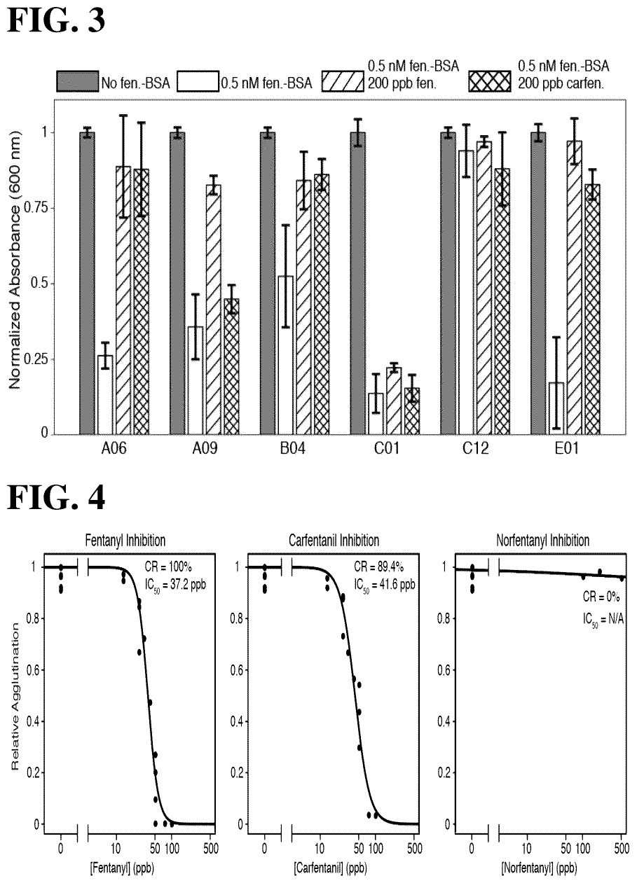

Cells of E. coli transformed with the surface display expression vector for each V_{HH} sequence were grown to an optical density (“OD600”) of 1.5 and incubated with 0.5 nM of fentanyl-BSA as the multivalent, agglutination inducing antigen. Absorbance at 600 nm was measured with a Hach DR3900 laboratory spectrophotometer, for each strain, after 30 minutes of incubation at room temperature (25 °C). Significant agglutination was observed in five of the six tested V_{HH} sequences, with the optical density of each agglutinating cell culture falling below half its initial value (FIG. 3).

Additionally, the ability of this agglutination assay reaction to be inhibited by opioids was determined by incubating cell cultures with 0.5 nM of fentanyl-BSA and 200 ppb of either unbound fentanyl or unbound carfentanil. For multiple single domain antibody sequences: A06 (SEQ ID NO: 10), B04 (SEQ ID NO: 21), and E01 (SEQ ID NO: 38), we observe noticeable cross-reactivity between fentanyl and carfentanil, as indicated by the ability of both compounds to inhibit agglutination by fentanyl-BSA. Furthermore, we observed more specific fentanyl binding for the A09 (SEQ ID NO: 15) variant, for which carfentanil did not significantly inhibit the agglutination reaction. Finally, while the C01 (SEQ ID NO: 24) variant exhibited strong agglutination in the presence of fentanyl-BSA, this reaction was not inhibited by either fentanyl or carfentanil at the tested concentrations. These results imply that the inhibition assay described here can be used as a test for the presence of fentanyl or carfentanil to concentrations of at least 200 ppb. Furthermore, the unique binding properties among these variants implies that tests with differing sensitivities or specificities to fentanyl can be developed by selecting a particular single domain antibody sequence.

To further probe the sensitivity and cross-reactivity of this competitive-binding-based fentanyl-detection assay, we measured the agglutination of the A06 strain, expressing (SEQ ID NO: 10) on its surface, at multiple concentrations of fentanyl, carfentanil, or norfentanyl (FIG. 4). Cells were concentrated three-fold and incubated with 1.5 nM of fentanyl-BSA conjugate, supplemented with the specified concentration of unbound opioid, and subsequently measured after a 30-minute incubation at room temperature. Strong inhibition of the agglutination reaction was observed for both fentanyl or carfentanil, with a similar IC₅₀, and high cross-reactivity (89.4%). However, no inhibition of agglutination was observed when incubating with norfentanyl. This result further demonstrated the utility of these E. coli-displayed sdAb sequences for the development of a fentanyl detection assay at low ppb concentrations.

### Example 3. Affinity Measurement of E. coli Surface-Displayed sdAbs

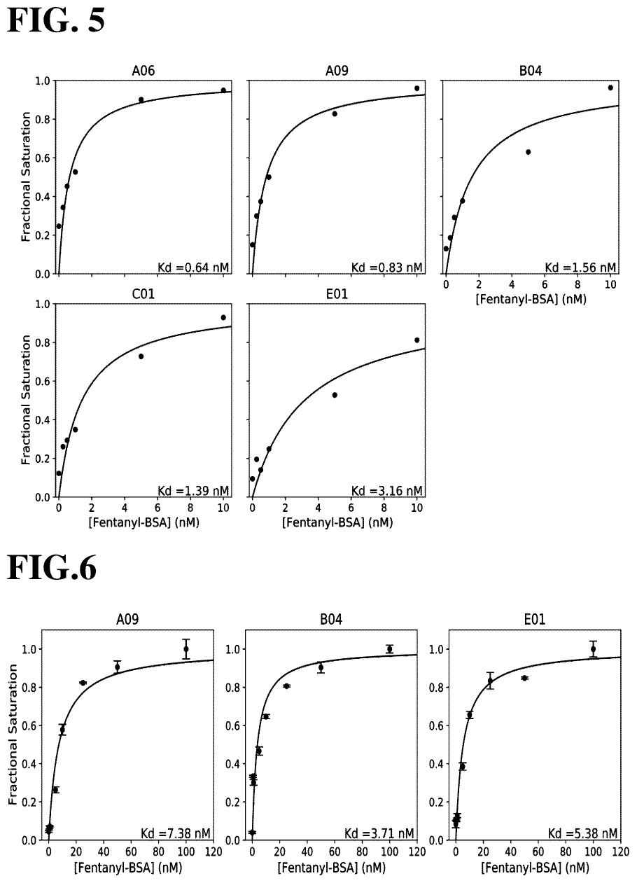

The binding affinity of membrane-anchored nanobodies was further quantified by whole-cell ELISA. Cells of E. coli displaying each V_{HH} were grown overnight and had their OD600 adjusted to 1 the next day. Cells were centrifuged, washed with phosphate-buffered saline (PBS), and resuspended with varying concentrations of fentanyl-BSA (spanning 0-10 nM) for 2 hours. Cells were again centrifuged, washed with PBS, then incubated with anti-BSA-FITC antibody for 2 hours (Thermo Fisher Scientific, Cat. No. PA1-29252). Cells were centrifuged and washed and resuspended in PBS a final time, and fluorescence was measured using a Tecan Infinite M200 plate reader. Binding affinity, K_{d}, was reported for each strain as the fentanyl-BSA concentration necessary for to reach half of the saturated signal (FIG. 5).

### Example 4. Affinity Measurement of V_{HH} Protein in Cell Free Extract of Cells Expressing V_{HH} Protein

Hexa-histidine-tagged nanobodies were expressed from the pADL-20c phagemid vector (Antibody Design Laboratories, Cat. No. PD0109) in an amber non-suppressive E. coli strain (MG1655). Cells were grown overnight, pelleted by centrifugation, resuspended in Working Solution from the CelLytic B Plus Kit for bacterial lysis (Sigma-Aldrich, Cat. No. CB0050), and vortexed. After a 15-minute lysis reaction, the lysate was centrifuged at 1,900×g for 15 minutes and the soluble protein was removed from the pelleted cell debris by pipetting and kept for affinity measurement. Microtiter plates were coated with varying concentrations of fentanyl-BSA conjugate at concentrations spanning 0-100 nM, at 4 °C overnight; the microtiter plates were then washed with PBS, and blocked with 1% casein in PBS (ThermoFisher Scientific, Cat. No. 37582) for two hours at room temperature. Wells were washed with PBS and then incubated with cell lysate for at 37 °C for 2 hours. Wells were again washed then incubated with an anti-6-His HRP antibody (BioLegend, Cat. No. 906109) for 2 hours at room temperature. After a final set of wash steps, antibody was quantified using TMB substrate (BioLegend. Cat. No. 421101) and signal was measured at 450 nm wavelength on a Tecan plate reader. Binding affinity (K_{d}) was reported for each strain as the fentanyl-BSA concentration necessary to reach half of the saturated signal (FIG. 6).

### Example 5. Real-Time Fentanyl Monitoring Through Continuous Agglutination Surveillance with Anti-Fentanyl sdAbs

We further developed the competitive-binding fentanyl assay into a semi-continuous testing method. Past cell-based agglutination assays have looked at the complete or incomplete formation of a cell pellet after 16 hours of incubation, typically in a microtiter plate. (See, e.g., Kylilis, N. et al., “Whole-cell biosensor with tunable limit of detection enables low-cost agglutination assays for medical diagnostic applications,” ACS Sensors, 4(2):370-378 (2019). doi.org/10.1021/acssensors.8b01163; and Riangrungroj, P. et al., Bever, C. S., Hammock, B. D., & Polizzi, K. M. (2019). “A label-free optical whole-cell Escherichia coli biosensor for the detection of pyrethroid insecticide exposure,” Scientific Reports, 9(1):1-9 (2019). doi.org/10.1038/s41598-019-48907-6).

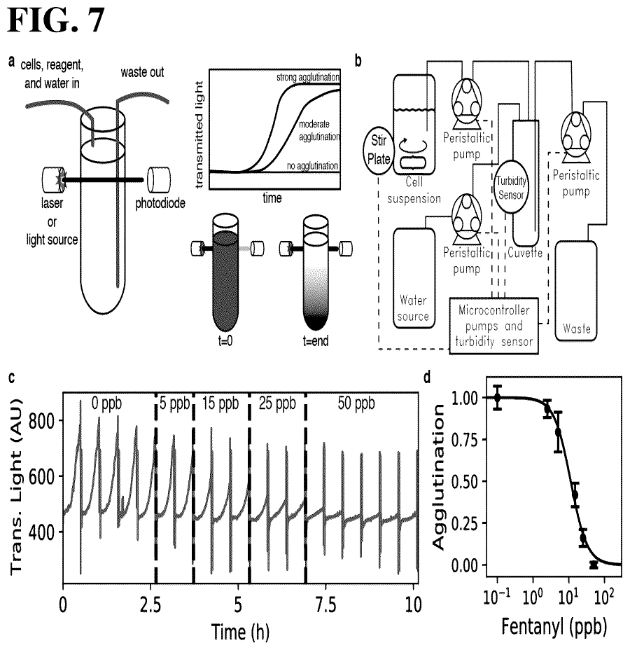

Here, however, we developed a system by which agglutination is instead monitored temporally. In this format, agglutination can be observed and quantified on the order of 1-45 minutes, e.g., a period of 10-30 minutes. In a reaction vessel, nanobody-displaying cells were mixed with a source to be monitored (e.g. water) and the transmitted light through a fixed point in the cell suspension was measured, as illustrated schematically in FIG. 7a. Non-agglutinating cells did not produce significant changes in transmitted light over short time scales, whereas agglutinating cells exhibited varying rates of agglutination dependent on how much of the binding or competing antigen was present.

A prototype was developed to adapt the approach to a continuous, real-time system by repeating the agglutination reaction in succession, as shown schematically in FIG. 7b, in an embodiment comprising an optional second vessel configured to receive and to contain the fluid aqueous sample to be analyzed (labeled in FIG. 7b, “Water source”). In such an embodiment the reaction vessel and other vessels (e.g., the cell suspension reservoir(s) and optional aqueous sample reservoir) can be configured to be airtight or open to the ambient atmosphere. Particular embodiments can include one or more inlets and/or outlets in any, or each, of the first vessel, second vessel, and/or reaction vessel.

The process, illustrated schematically in FIG. 7b, includes three simple phases, whereby: (i) fluid propellers, e.g., pumps, such as but not limited to, peristaltic pumps, move a cell suspension and monitored water into a reaction cuvette, (ii) the turbidity, or other parameter correlatable to the level of agglutination, of the cell suspension is measured over a selected a continuous time course of at least about 1 to 45 minutes (e.g., 30-minute period), (iii) fluid propellers, e.g., pumps, such as but not limited to, peristaltic pumps, remove the contents from the reaction cuvette to waste, the cuvette is washed with source water if necessary, and cells are resuspended via stir plate before the process repeats.

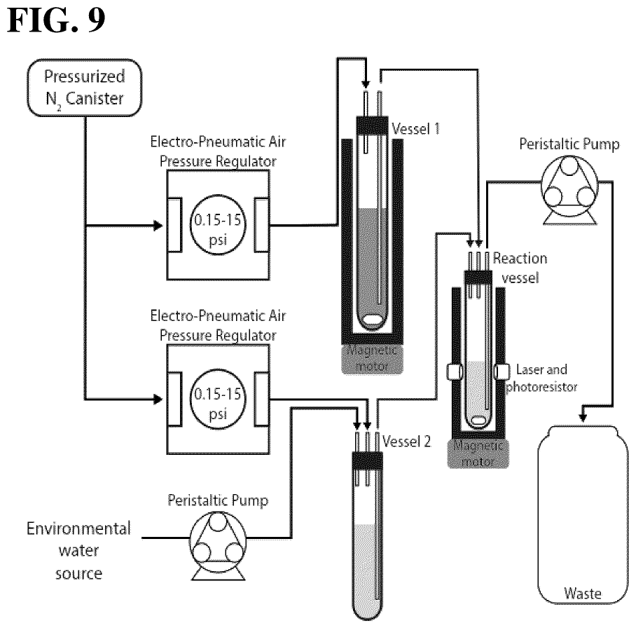

Another embodiment of a continuous, real-time system was also developed with pressurized inert gas directed into the headspace of the respective cell suspension reservoir(s) and the aqueous sample reservoir, to control the flow of agglutinating agent and fluid aqueous sample into the reaction vessel, as shown schematically in FIG. 9. In such an embodiment the reaction vessel and other vessels (e.g., the cell suspension reservoir(s) and optional aqueous sample reservoir) are configured to be airtight. In FIG. 9, this embodiment includes the optional aqueous sample reservoir (i.e., the second vessel; in FIG. 9, labeled “Vessel 2”) configured to receive and to contain the fluid aqueous sample to be analyzed (labeled in FIG. 9, “Environmental water source”). The process consists of three simple phases, whereby: (i) an inert gas (e.g., dinitrogen, helium, argon, neon, xenon, or the like) moves a cell suspension (living cells or fixed cells, e.g., by the process of fixation described herein) contained in the cell suspension reservoir or first vessel (in FIG. 9, labeled “Vessel 1”) and the water to be continuously monitored (in “Vessel 2”), through a dipleg in each of Vessel 1 and Vessel 2, and through connecting lines into the reaction vessel, (ii) a parameter correlatable to the level of agglutination (e.g., turbidity, transmission, scattering, or absorbance of light) is measured in a preselected portion of the reaction mixture over a continuous time course; in FIG. 9, a laser and photoresistors are schematically shown for measuring the selected parameter, but other parameters can be selected, e.g., parameters measurable with a piezoelectric detector or a surface plasmon resonance sensor. The parameter is measured over a selected a continuous time course of at least about 1 to 45 minutes (e.g., 30 min period), (iii) a fluid propeller, e.g., a pump, such as but not limited to, a peristaltic pump, removes the contents from the reaction vessel or cuvette to waste, the reaction vessel is washed with source water if necessary, and cells are resuspended via stir plate before the process repeats. In the embodiment illustrated in FIG. 9, a peristaltic pump is also used to move the aqueous sample or environmental water into the aqueous sample reservoir. In FIG. 9, magnetic stirring motors are shown under the cell suspension reservoir and the aqueous sample reservoir, with magnetic stir bars in the first vessel and the reaction vessel, but any other suitable type of stirrer can be used instead, e.g., a gentle shaker, a propeller, or sparging with inert gas bubbles; stirring is typically at about 5-300 rpm. The microcontroller is present and operates in this embodiment, as in other embodiments, but is not illustrated in FIG. 9. In addition, in this and similar embodiments employing the pressure of an inert gas (e.g., N₂, He, Ar, Ne, or Xe) in the vessel headspace to move fluid around in the system, the microcontroller can be configured and programmed to operate the valves and gas pressure regulators of the gas plumbing system.

A description of the specific components of the embodiments shown in FIG. 7b and FIG. 9, as well as alternative embodiments, are described in more detail below.

In FIG. 7b, the “cell suspension” reservoir (designated “Vessel 1,” in FIG. 9) refers to any combination of one or more cell suspension vessels or reservoirs, containing the cells displaying the nanobody on their surface, and any additional reagents for necessary for agglutination. This cell suspension reservoir(s) can contain living cells or fixed cells (e.g., by the process of fixation described below). For either fixed or living cells, agitation of the culture is necessary to preserve culture homogeneity and prevent cell settling. However, it was observed that constant agitation of fixed cells, for example by a magnetic stir bar, dampens the agglutination phenotype over time, and therefore stirring is better performed intermittently, just before pulling fluid from the reservoir, rather than continuously. While we used a magnetic stir bar to prevent cell settling, any number of alternative agitation methods can be used, including shaking or an impeller. Additionally, if live cells are used, proper culture maintenance, such as media addition, waste removal, and temperature control, need to be used. Fixed cells, on the other hand, are stable at room temperature, though cold storage can improve their longevity. The cell suspension can also contain any necessary reagents for agglutination; this can include agglutinating agents necessary for competitive-binding assays (e.g. fentanyl-BSA in the case of fentanyl detection), chemicals to prevent microbial contamination (such as sodium azide or an antibiotic or a bacteriostatic compound), or coagulants and/or other additives to enhance the agglutination reaction, such as, but not limited to, polyethylene glycol (PEG) or polyamines. However, the additional reagents do not necessarily need to be contained within the cell suspension and can be kept, optionally, in a separate vessel or reservoir and mixed in at a later point in the process.

In FIG. 7b, the “Water source,” or in FIG. 9 “Environmental water source,” schematically represents the sample to be analyzed using the inventive antigen-binding proteins, device, and/or method. This sample can include a batch water sample, or an inline plug-in to a continuously flowing aqueous stream, which can be received and contained by the optional second vessel, or which can be introduced directly into the reaction vessel via a sample inlet, as desired. We have observed the inventive fentanyl assay to be functional on a range of water sources, including tap water, river water, algal pond water, and dairy manure lagoon water. Pretreatment or filtration of the aqueous sample can optionally be employed, depending on the pH, salinity, or solids content of the aqueous sample. However, we have observed good, relatively unchanged agglutination with salinities of up to 2 M NaCl and in a sample pH range of pH 5-9. If pre-treatment of the water is necessary, this can be done in a separate vessel or reservoir prior to the agglutination reaction, or any necessary buffers and additives can be supplemented into the cell suspension reservoir.

In the embodiment illustrated in FIG. 7b, peristaltic pumps are shown for moving fluid throughout the system, however, any of various fluid transfer technologies can be used instead. Other mechanical pumps, such as diaphragm pumps, syringe pumps, etc., can be used in place of peristaltic pumps. Alternatively, simply pipetting/bulk liquid movement can be used in lieu of any pumps, if desired. As previously mentioned, gas pressure in the first vessel and second vessel headspaces can also be used to control fluid flow through the connecting lines of the system into the reaction vessel (see, e.g., FIG. 9).

In FIG. 7b, a current sensing element serves as a turbidity sensor, using a photoresistor to measure changes in the amount of light transmitted by a laser (Digi-Key VLM-650-03 LPA) across a fixed point in the cell suspension. However, the parameter to be measured can be any parameter that can be correlated to the level of agglutination. Alternatively, there are multiple other methods of measuring cell density, or aggregate formation, that can be used, as is more generally illustrated in FIG. 9, including measurement of transmitted light, absorbance of light, scattered light, fluorescence, flow cytometry, microscopy, or electrical impedance. In addition to the embodiment represented schematically in FIG. 7b, we employed an embodiment including fluorescence detection: labeling fixed cells with a green fluorescent protein, exciting the cell suspension with 470-nm wavelength light and using a long-pass filter to only measure the emitted signal. A fluorescence detection approach is particularly useful when analyzing high-turbidity water samples, which can adversely affect a transmitted light measurement. Samples were also tested by flow cytometry, and we found that changes in the forward scatter and side scatter distribution of a cell population were changed when agglutination occurred. These changes were measurable by flow cytometry on the order of less than 1 minute.

In FIG. 7b, the “Cuvette” refers to any container (i.e., a “reaction vessel,” as designated in FIG. 9) within which the agglutination reaction occurs and is measured. This container can be a culture tube, a cuvette (glass or plastic), or a centrifuge tube. Depending on the liquid transfer method and number of cell(s), reagent(s), and water sources, the “cuvette” may have inlet and outlet fluidics. It is important that these fluidics do not impede the sensing element, for example, by impeding the path of a light source. Lastly, it is important that the cell suspension, reagents, and water source are adequately mixed in the “Cuvette.” If this cannot be achieved effectively through the liquid transfer step, an in-cuvette mixer can be implemented.

Finally, the “Microcontroller” represented schematically in FIG. 7b, refers to any digital computational device used to control the electronic and/or automated components of the device, including pumps and the sensing element. In one exemplary embodiment of the microcontroller, an Arduino Uno REV3 (Arduino A000066) and Raspberry Pi 4 (CanaKit P14-1 GB-STR32F-C4-BLK) were employed, from which modbus commands were sent to control the pumps and the voltages across the photoresistor that were measured to determine turbidity. Measured voltages were stored in an on-device SQLite database and were periodically synced to a remote server. Other embodiments of microcontrollers, communications protocols, and data storage can vary depending on the hardware used throughout the device or system. While the embodiment illustrated schematically in FIG. 7a-b does not include switching of flow paths, in some other embodiments, path switching can be desirable. For example, in one such embodiment, a multi-parameter test can contain multiple cell suspension reservoirs (i.e., a plurality of the first vessel), each with a sensor strain sensitive to a different specific analyte, and this embodiment of the system is capable of drawing from each of different cell suspension reservoirs. In some such embodiments the path switching can be automated, including the use of automated valves directed by the microcontroller. As mentioned above, in embodiments employing an inert gas in the vessel headspace to move fluid around in the system, the microcontroller can be configured and programmed to also operate the valves and gas pressure regulators of the gas plumbing system.

In the embodiments illustrated schematically in FIG. 7a-b and FIG. 9, all the vessels and the tubing of the connecting lines must be cleaned or replaced between runs. When cleaning tubing of the connecting lines, the cell suspension vessel (“first vessel”), water source or sample vessel (i.e., “second vessel”), and/or the reaction vessel, are replaced with vessels containing a cleaning agent. If no contamination or clogging of the tubing was observed, simply running a 70% ethanol solution through the system for three cycles is sufficient to clean the system for the next experimental or analytical run. Each cleaning cycle consists of filling the vessel (e.g., cuvette or reaction vessel), allowing this vessel containing the cleaning solution to sit for about 1 minute, and then removing the contents into the waste receptacle. If bacterial or fungal contamination was observed upstream of any vessel (e.g., in the first vessel, in the second vessel, or in the tubing of the connecting lines), then a bleach solution (10%) or detergent solution (e.g., Alconox, Millipore Sigma Z742914, 1% w/v) should be used before the ethanol rinse. A single bleach cycle is sufficient if no clogging or biofilm formation was observed with contamination, however, if clogging or biofilms are observed, detergent should be cycled through the system until it is removed. After any bleach, detergent, or ethanol rinses are completed, two cycles of sterile water should be run to the system to remove any residual cleaning reagents, before a new experimental or analytical run of the system. In some embodiments this cleaning routine can be automated, e.g., under the control of the microcontroller, without the need for user intervention between runs.

In FIG. 7(c), we have demonstrated an exemplary 30-minute period for the inventive continuous agglutination assay method for detecting an analyte of interest, in this exemplary embodiment, for the detection of fentanyl, however, the incubation period can be increased or decreased based on the agglutination reaction time or desired sampling frequency. In fact, we observed distinguishable transmitted light values between samples with and without 15 ppb fentanyl within 13 minutes of setting up the agglutination reaction, which implied that a 13-minute sampling frequency can be used in this embodiment. In cases where the desired sampling frequency is higher than the minimum response time of the agglutination reaction, the desired frequency can be achieved by deploying staggered reactions in parallel.

Further testing with a competitive-binding embodiment of the continuous assay method, with known competitive concentrations of fentanyl added, showed that the analyte concentrations can be estimated from the amount of agglutination at 30 minutes (FIG. 7d). In FIG. 7d, the agglutination value presented on the y-axis is normalized such that a value of 1 is equal to the mean transmitted light of three independent agglutinating cultures with no added fentanyl, which is the case wherein we expect the highest degree of agglutination, TLₘₐₓ; and a value of 0 is equal to the mean transmitted light of three independent agglutinating cultures with no addition of agglutinating agent (i.e., fentanyl-BSA conjugate in this embodiment), which is the case wherein we expect no agglutination, TLₘᵢₙ. Therefore, agglutination can be quantified by the formula:

$$Agglutination = \frac{TL - {TL}_{min}}{{TL}_{max} - {TL}_{min}} ,$$

whereby TL is the measured transmitted light of the sample. For different detection modalities, the agglutination formula can be generalized to the measurement taken (X), normalized relative to the maximum and minimum control levels (Xₘₐₓ and Xₘᵢₙ, respectively), such that:

$$Agglutination = \frac{X - {X}_{min}}{{X}_{max} - {X}_{min}} ,$$

whereby X is the measured value. For example, in embodiments in which fluorescence is measured by a fluorescence detector, X can be fluorescence intensity of the reaction mixture, or a portion of the reaction mixture. In an embodiment in which conductivity is measured by a conductivity detector, X can be the conductance of the reaction mixture, or a portion thereof. For embodiments in which flow cytometry or particle-size-based measurement(s) are made by appropriate instruments, X can be the mean forward scatter value for a population of cells in the reaction mixture or a sample portion thereof.

The quantification method presented above in FIG. 7d is based on comparing the measured transmitted light value at a constant time after the agglutination reaction is started, however, other methods of measuring the reaction and analyzing the data can be used instead. One such method can involve looking at the rate of agglutination, whereby an exponential or linear curve is fit to each agglutination reaction, with the slope or other fit parameters being mapped back to the added fentanyl concentration. Ultimately, this approach for measuring agglutination enables the capture of temporal data, enabling faster measurement and more nuanced determination of analyte concentration than a binary, microtiter plate-based assay.

To ensure the stability of the cellular reagent for the continuous agglutination assay method, cells were fixed with paraformaldehyde (PFA) prior to storage and use in the test. Briefly, the protocol of Jose (2005) was adapted for fixing the cells, with sdAb-displaying cells being cultured overnight in LB medium at 37 °C, washed three times with phosphate buffered saline (PBS), incubated with 4% (v/v) PFA for 20 minutes at room temperature, washed three more times in PBS, and resuspended at half the culture volume in PBS for a final OD600 of about 3. Cells fixed in this manner were stable at room temperature for at least one month. (See, Jose, J. et al., “Bacterial surface display library screening by target enzyme labeling: Identification of new human cathepsin G inhibitors,” Analytical Biochemistry, 346:258-267 (2005). doi.org/10.1016/j.ab.2005.08.019).

### Example 6. Real-Time Protein Monitoring Through Continuous Agglutination Surveillance with Anti-IgG sdAbs

To confirm that the inventive continuous sensing approach can be used to monitor not just small molecule analytes, but also protein analytes of interest, we deployed the inventive continuous agglutination assay method to detect a previously published anti-mouse secondary sdAb with a specific affinity to the mouse Kappa (κ) fragment antigen-binding (Fab) isotype (ID TP1170); this anti-mouse Kappa (κ) sdAb was first described by Pleiner et al. (2018), and we cloned it into our E. coli nanobody display vector. (See, Pleiner, T. et al., “A toolbox of anti-mouse and anti-rabbit IgG secondary nanobodies,” Journal of Cell Biology, 217(3):1143-1154 (2018). doi.org/10.1083/jcb.201709115).

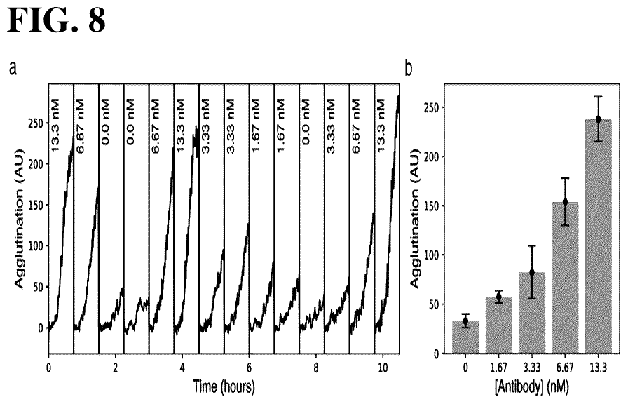

Fixed E. coli cells displaying this nanobody were then integrated into the continuous agglutination assay and incubated with samples containing varying concentrations of a purified mouse IgG1, κ isotype control antibody (BioLegend, Cat. No. 400101). We observed increased agglutination in the presence of higher concentrations of the control antibody, with concentrations down to 1.67 nM detectable within 45 minutes of introduction to the fixed cells (FIG. 8a). Likewise, we observe some ability to quantify the concentration of the introduced antibody between the 1.67-13.3 nM range (FIG. 8b). We foresee this type of agglutination assay being useful and expandable for any protein target for which sdAbs can be developed. For protein targets with multiple binding sites (e.g., the Kappa isotype on an antibody), a direct agglutination approach can be deployed. And for targets with single binding sites (e.g., fentanyl), a competitive-binding assay can be developed.

1-20. (canceled)

21. A continuous agglutination assay method for detecting an analyte of interest, comprising:
(a) mixing in a reaction vessel, a fluid aqueous suspension of host cells that display on their surfaces a plurality of recombinant antigen-binding proteins that specifically bind an analyte of interest, with a fluid aqueous sample, in a reaction mixture with an agglutinating agent comprising the analyte of interest, or an analyte conjugate, under conditions of temperature and pH that permit binding of the analyte or the analyte conjugate, by the antigen-binding protein and agglutination of the cells, in the presence of the analyte of interest;
(b) measuring a parameter in a preselected portion of the reaction mixture over a continuous time course;
(c) correlating the change in the measured parameter over the time course with the level of agglutination; and
(d) normalizing the level of agglutination relative to maximum and minimum control levels of agglutination to obtain a normalized value;
wherein the normalized value reflects the concentration of the analyte of interest in the aqueous sample over the continuous time course.

22. The continuous agglutination assay method according to claim 21, wherein the step of measuring the level of agglutination in the reaction mixture is over a continuous time course of at least about 1 to 45 minutes.

23. The continuous agglutination assay method according to claim 21, wherein the step of measuring the level of agglutination in the reaction mixture is through a fixed distance in the preselected portion of the reaction mixture in the reaction vessel.

24. The continuous agglutination assay method according to claim 21, wherein the step of measuring the level of agglutination in the reaction mixture comprises measuring the turbidity of the reaction mixture.

25. The continuous agglutination assay method according to claim 21, wherein the step of measuring the level of agglutination in the reaction mixture comprises detecting the transmission, scattering, or absorbance of light.

26. The continuous agglutination assay method according to claim 21, wherein the preselected portion of the reaction mixture is in the upper half- to upper third-portion.

27. The continuous agglutination assay method according to claim 21, further comprising adding a series of known concentrations of the analyte, or an analyte conjugate, to the reaction mixture to compete with the agglutinating agent, during a series of one or more further continuous time courses.

28. The continuous agglutination assay method according to claim 21, wherein the host cells are fixed prior to use in the method.

29. The continuous agglutination assay method according to claim 21, wherein the analyte of interest is fentanyl or carfentanil, and wherein the agglutinating agent comprises fentanyl, carfentanil, and/or a conjugate of either.

30. The continuous agglutination assay method according to claim 29, wherein the plurality of recombinant antigen-binding proteins comprises one or more antigen-binding proteins that specifically bind fentanyl or carfentanil.

31. An automated system for continuous agglutination assay, comprising:
(a) a first vessel configured to receive and to contain, a fluid aqueous suspension comprising host cells that display on their surfaces a plurality of recombinant antigen-binding proteins that specifically bind an analyte of interest, and, wherein the fluid aqueous suspension optionally comprises an antibiotic or bacteriostatic compound, a coagulant and/or an agglutination-enhancing additive;
(b) an optional second vessel configured to receive and to contain a fluid aqueous sample to be analyzed;
(c) a reaction vessel fluidly connected to the first vessel and to the optional second vessel by connecting lines, wherein the reaction vessel is configured to automatically receive via the connecting lines, a predetermined volume of the fluid aqueous suspension from the first vessel and a predetermined volume of the fluid aqueous sample, optionally from the second vessel, or directly from a sample inlet, and to contain a reaction mixture comprising the host cells and the aqueous sample;
(d) a sensor configured to measure a parameter correlatable to the level of agglutination in the reaction mixture in the reaction vessel, in response to a measurement signal, and to transmit the thusly acquired measurement data;
(e) a waste outlet, or optionally a waste vessel, fluidly connected to the reaction vessel by a connecting line;
(f) a plurality of fluid propellers each configured to impel fluid flow through the connecting lines in response to a signal, wherein at least one fluid propeller is upstream from the reaction vessel, and at least one fluid propeller is downstream from the reaction vessel;
(g) a microcontroller configured and programmable to signal the plurality of fluid propellers to initiate fluid flow, impel fluid flow, or cease fluid flow, through the connecting lines:
(i) from the first vessel, and from the optional second vessel or the sample inlet, to the reaction vessel;
(ii) from the reaction vessel to the waste outlet, or optionally to the waste vessel; and
wherein the microcontroller, or optionally, a separate microcontroller, is further configured and programmable to transmit the measurement signal to the sensor, and to collect the measurement data from the sensor.

32. The automated system for continuous agglutination assay of claim 31, comprising the optional second vessel, configured to receive and to contain the fluid aqueous sample to be analyzed.

33. The automated system for continuous agglutination assay according to claim 31, wherein the analyte of interest is fentanyl or carfentanil, and the plurality of recombinant antigen-binding proteins comprises one or more antigen-binding proteins that specifically bind fentanyl or carfentanil.

34. The automated system for continuous agglutination assay according to claim 31, wherein the reaction vessel, the first vessel, and/or the optional second vessel, further comprise a stirrer.

35. The automated system for continuous agglutination assay according to claim 31, wherein the plurality of fluid propellers comprise one or more pump heads.

36. The automated system for continuous agglutination assay according to claim 31, wherein the system is airtight, and the plurality of fluid propellers comprises one or more gas regulator(s) for regulating the pressure of an inert gas in the headspace of the first vessel, of the optional second vessel, and of the reaction vessel.

37. (canceled)

38. The continuous agglutination assay method according to claim 21, wherein the analyte of interest is a cell membrane-surface antigen.

39. The continuous agglutination assay method according to claim 21, wherein the analyte of interest is a protein target.

40. The continuous agglutination assay method according to claim 21, wherein the analyte of interest comprises a small molecule analyte.

41. The continuous agglutination assay method according to claim 21, wherein the analyte of interest is a glycoprotein.

42. The automated system for continuous agglutination assay according to claim 31, wherein the analyte of interest is a cell membrane-surface antigen.

43. The automated system for continuous agglutination assay according to claim 31, wherein the analyte of interest is a protein target.

44. The automated system for continuous agglutination assay according to claim 31, wherein the analyte of interest comprises a small molecule analyte.

45. The automated system for continuous agglutination assay according to claim 31, wherein the analyte of interest is a glycoprotein.


SEQUENCE LISTING (64 sequences)

SEQ ID NO: 1 - protein, 135 aa, synthetic construct
QVQLVESGGGLAQAGGSLRLSCTGSGRTSNVNIVGWFRQPPGKEREFVAAIRWRDLRTWYPYYADSVKGRFALSTNNAANTVFLQMNSLKPEDTAVYYCACAARARPRTGEWDTSVTGARDYDYWGQGTQVTVSS

SEQ ID NO: 2 - protein, 9 aa, synthetic construct
RTSNVNIVG

SEQ ID NO: 3 - protein, 11 aa, synthetic construct
AIRWRDLRTWY

SEQ ID NO: 4 - protein, 24 aa, synthetic construct
CAARARPRTGEWDTSVTGARDYDY

SEQ ID NO: 5 - protein, 127 aa, synthetic construct
EVQLVESGGGLVQVGGSLRLSCTASGRAYESPIAGWFRQAPGKEREFVATLLWSSGSTDYADPVKGRFTISKGTTDNMVYLQMNNLKADDTAIYYCAAATVFRGRLASRLPTSYDYWGQGTQVTVSS

SEQ ID NO: 6 - protein, 9 aa, synthetic construct
RAYESPIAG

SEQ ID NO: 7 - protein, 11 aa, synthetic construct
TLLWSSGSTDY

SEQ ID NO: 8 - protein, 19 aa, synthetic construct
AATVFRGRLASRLPTSYDY

SEQ ID NO: 9 - protein, 127 aa, synthetic construct
QVQLVESVGGLVQVGGSLRLSCTASGRAYESPIAGWFRQAPGKEREFVATLLWSSGSTDYADPVKGRFTISKGTTDNMVYLQMNNLKADDTAIYYCAAATVFRGRLASRLPTSYDYWGQGTQVTVSS

SEQ ID NO: 10 - protein, 135 aa, synthetic construct
EVQLVESGGGLAQAGGSLRLSCTGSGRTSNVNIVGWFRQPPGKEREFVAAIRWRDLRTWYPYYADSVKGRFALSTNNAANTVFLQMNSLKPEDTAVYYCACAARARPRTGEWDTSVTGARDYDYWGQGTQVTVSS

SEQ ID NO: 11 - protein, 127 aa, synthetic construct
QVQLVESVGGLVQVGGSLRLSCTASGRAYSSPIVGWFRQAPGKEREFVATLFWHSGSTDYGDPVKGRFTVSRATADNMVYLQMNSLKAEDTAIYYCAAATIFRGRLGYRLPTAYDYWGQGTQVTVSS

SEQ ID NO: 12 - protein, 9 aa, synthetic construct
RAYSSPIVG

SEQ ID NO: 13 - protein, 11 aa, synthetic construct
TLFWHSGSTDY

SEQ ID NO: 14 - protein, 19 aa, synthetic construct
AATIFRGRLGYRLPTAYDY

SEQ ID NO: 15 - protein, 127 aa, synthetic construct
QVQLVESGGGLVQVGGSLRLSCTASGRAYESPIAGWFRQAPGKEREFVATLLWSSGSTDYADPVKGRFTISKGTTDNMVYLQMNNLKADDTAIYYCAAATVFRGRLASRLPTSYDYWGQGTQVTVSS

SEQ ID NO: 16 - protein, 119 aa, synthetic construct
QVQLVESVGGWVQAGGSLRLSCTASGSIFSINDMAWYRQATGKQRERVAVLVRNYTHYLDSVKGRFTISRDNANAKIALNLQMNSLKPEDIAVYFCNANTFSRAGPEYWGQGTQVTVSS

SEQ ID NO: 17 - protein, 9 aa, synthetic construct
SIFSINDMA

SEQ ID NO: 18 - protein, 9 aa, synthetic construct
VLVRNYTHY

SEQ ID NO: 19 - protein, 11 aa, synthetic construct
ANTFSRAGPEY

SEQ ID NO: 20 - protein, 135 aa, synthetic construct
QVQLVESGGGLAQAGGSLRLSCTGSGRTSNVNIVGWFRQPPGKEREFVAAIRWRDLRTWYPYYADSVKGRFALSTNNAANTVFLQMNSLKPEDTAVYYCACAARARPRTGEWDTSVTGARDYDYWGQGTLVTVSS

SEQ ID NO: 21 - protein, 127 aa, synthetic construct
QVQLVESGGGLVQVGGSLRLSCTASGRAYSSPIVGWFRQAPGKEREFVATLFWHSGSTDYGDPVKGRFTVSRATADNMVYLQMNSLKAEDTAIYYCAAATIFRGRLGYRLPTAYDYWGQGTQVTVSS

SEQ ID NO: 22 - protein, 135 aa, synthetic construct
QVQLVESGGGLAQAGGSLRLSCTGSGRTSNVNIVGWFRQPPGKEREFVAAIRWRDLRTWYPYYADSVKGRFALSTNNAANTVFLQMNSLKPVDTAVYYCACAARARPRTGEWDTSVTGARDYDYWGQGTQVTVSS

SEQ ID NO: 23 - protein, 127 aa, synthetic construct
EVQLVESGGGLVQVGGSLRLSCTASGRAYSSPIVGWFRQAPGKEREFVATLFWHSGSTDYGDPVKGRFTVSRATADNMVYLQMNSLKAEDTAIYYCAAATIFRGRLGYRLPTAYDYWGQGTLVTVSS

SEQ ID NO: 24 - protein, 128 aa, synthetic construct
QVQLVESGGGLVQAGGSLSLSCSASGRAFRSYVVGWFRQAPGKERYFVASINWGSLRTTYGDGVKGRFTISRDAAKNTVNLQMNSLKPEDTAVYYCAAMSGSSYATTPRSSADEYDYWGQGTLVTVSS

SEQ ID NO: 25 - protein, 9 aa, synthetic construct
RAFRSYVVG

SEQ ID NO: 26 - protein, 11 aa, synthetic construct
SINWGSLRTTY

SEQ ID NO: 27 - protein, 20 aa, synthetic construct
AMSGSSYATTPRSSADEYDY

SEQ ID NO: 28 - protein, 127 aa, synthetic construct
QVQLVESGGGLVQVGGSLRLSCTASGRAYESPIAGWFRQAPGKEREFVATLLWSSGSTDYADPVKGRFTISKGATDNMVYLQMNNLKADDTAIYYCAAATVFRGRLASRLPTSYDYWGQGTLVTVSS

SEQ ID NO: 29 - protein, 127 aa, synthetic construct
EVQLVESGGGLVQAGGSLRLSCAASGRTFSNYVMGWFRLAPGKEREFVAAIHWNIGKTIYADSAKGRFTISRDNAKNTVYLQMNSLKPEDTALYYCVATWRGASIDMPTTANEYRYRGQGTLVTVSS

SEQ ID NO: 30 - protein, 9 aa, synthetic construct
RTFSNYVMG

SEQ ID NO: 31 - protein, 11 aa, synthetic construct
AIHWNIGKTIY

SEQ ID NO: 32 - protein, 19 aa, synthetic construct
ATWRGASIDMPTTANEYRY

SEQ ID NO: 33 - protein, 117 aa, synthetic construct
QVQLVESGGGLVQPGGSLRLSCAASGFTLGDYDIGWFRQAPGKERVGVSCATRREGNTWYADSVKGRFVISRDNAKNTVYLQMRSLKFEDTAVYYCAVPCWGRGRYWGQGTQVTVSS

SEQ ID NO: 34 - protein, 9 aa, synthetic construct
FTLGDYDIG

SEQ ID NO: 35 - protein, 11 aa, synthetic construct
CATRREGNTWY

SEQ ID NO: 36 - protein, 9 aa, synthetic construct
VPCWGRGRY

SEQ ID NO: 37 - protein, 128 aa, synthetic construct
QVQLVESGGGLVQAGGSLSLTCSASGRAFRSYVVGWFRQAPGKERYFVASINWGSLRTTYGDGVKGRFTISRDVAKNTVNLQMNSLKPEDTAVYYCAAMSGSSYATTPRSSADEYDYWGQGTQVTVSS

SEQ ID NO: 38 - protein, 135 aa, synthetic construct
EVQLVESGGGLAQAGGSLRLSCAGSGRTSNVNIVGWFRQPPGKEREFVAAIRWRDLRTWYPYYADSVKGRFALSTNNAANTVFLQMNSLKPEDTAVYYCACAARARPRTGEWDTSVTGARDYDYWGQGTQVTVSS

SEQ ID NO: 39 - protein, 124 aa, synthetic construct
QVQLVESGGGLVQPGGSLRLSCVASGNTYGIDVMGWYRQAPGEQRELVAIITSGGSTNYADSVKGRFTISAEYAKNTVYLQMNSLTSEDTAVYTCHAHESRWSMRGKTREYDYWGQGTQVTVSS

SEQ ID NO: 40 - protein, 9 aa, synthetic construct
NTYGIDVMG

SEQ ID NO: 41 - protein, 10 aa, synthetic construct
IITSGGSTNY

SEQ ID NO: 42 - protein, 17 aa, synthetic construct
AHESRWSMRGKTREYDY

SEQ ID NO: 43 - protein, 124 aa, synthetic construct
QVQLVESGGGLAQAGGSLRLSCTGSGRTSNVNIVGWFRQPPGKEREFVAAIRWRDLRTWYPYYADSVKGRFALSTNNAANTVFLQMNSLKPEDTAVYYCAARQGSNWYLHPDYWGQGTLVTVSS

SEQ ID NO: 44 - protein, 13 aa, synthetic construct
ARQGSNWYLHPDY

SEQ ID NO: 45 - protein, 135 aa, synthetic construct
EVQLVESGGGLARAGGSLRLSCTGSGRTSNVNIVGWFRRPPGKEREFVAAIRWRDLRTWYPYYADSVKGRFALSTNNAANTVFLQMNSLKPEDTAVYYCACAARARPRTGEWDTSVTGARDYDYWGQGTQVTVSS

SEQ ID NO: 46 - protein, 135 aa, synthetic construct
EVQLVESGGRLVQAGGSLRLSCTGSGRTSNVNIVGWFRQPPGKEREFVAAIRWRDLRTWYPYYADSVKGRFALSTNNAANTVFLQMNSLKPEDTAVYYCACAARARPRTGEWDTSVTGARDYDYWGQGTQVTVSS

SEQ ID NO: 47 - protein, 135 aa, synthetic construct
QVQLVESVGGLVQAGGSLRLSCTGSGRTSNVNIVGWFRQPPGKEREFVAAIRWRDLRTWYPYYADSVKGRFALSTNNAANTVFLQMNSLKPEDTAVYYCACAARARPRTGEWDTSVTGARDYDYWGQGTQVTVSS

SEQ ID NO: 48 - protein, 135 aa, synthetic construct
EVQLVESGGGLAQAGGSLRLSCTGSGRTSNVNIVGWFRQPPGKEREFVAAIRWRDLRTWYPYYADSVKGRFALSTNNAANTVFLQMNSLKPEDTAVYYCACAARARPRTGEWDTSVTGARDYDYWGQGTLVTVSS

SEQ ID NO: 49 - protein, 127 aa, synthetic construct
EVQLVESGGGLVQVGGSLRLSCTASGRAYSSPIVGWFRQAPGKEREFVATLFWHSGSTDYGDPVKGRFTVSRATADNMVYLQMNSLKAEDTAIYYCAAATIFRGRLGYRLPTAYDYWGQGTQVTVSS

SEQ ID NO: 50 - protein, 128 aa, synthetic construct
EVQLVESGGGLVQAGGSLSLSCSASGRAFRSYVVGWFRQAPGKERYFVASINWGSLRTTYGDGVKGRFTISRDVAKNTVNLQMNSLKPEDTAVYYCAAMSGSSYATTPRSSANEYDYWGQGTLVTVSS

SEQ ID NO: 51 - protein, 20 aa, synthetic construct
AMSGSSYATTPRSSANEYDY

SEQ ID NO: 52 - protein, 127 aa, synthetic construct
EVQLVESGGGLVQVGGSLRLSCTASGRAYSSPIVGWFRQAPGKEREFVATLFWHSGSTDYGDPVKGRFTVSRATADNMVYLQMNSLKAEDTAIYYCAAATIFRGRLGYRLPTAYDYWGRGTQVTVSS

SEQ ID NO: 53 - protein, 127 aa, synthetic construct
QVQLVESGGGLVQAGGSLRLSCTASGRAYSSPIVGWFRQAPGKEREFVATLFWHSGSTDYGDPVKGRFTVSRATADNMVYLQMNSLKAEDTAIYYCAAATIFRGRLGYRLPTAYDYWGQGTQVTVSS

SEQ ID NO: 54 - protein, 119 aa, synthetic construct
QVQLVESGGGLVQPGGSLTLSCVVSGIRFSVHTVGWYRQGPGSQREFVASAGPHSRTDYAHFVRGRFTISRDEAKNTAYLQMANLQPEDTAVYYCARDGTSPIPEFDCWGQGTQVTVSS

SEQ ID NO: 55 - protein, 9 aa, synthetic construct
IRFSVHTVG

SEQ ID NO: 56 - protein, 10 aa, synthetic construct
SAGPHSRTDY

SEQ ID NO: 57 - protein, 12 aa, synthetic construct
RDGTSPIPEFDC

SEQ ID NO: 58 - protein, 22 aa, synthetic construct
MDMRVPAQLLGLLLLWLRGARC

SEQ ID NO: 59 - protein, 20 aa, synthetic construct
MEAPAQLLFLLLLWLPDTTG

SEQ ID NO: 60 - protein, 19 aa, synthetic construct
MEWTWRVLFLVAAATGAHS

SEQ ID NO: 61 - protein, 20 aa, synthetic construct
METPAQLLFLLLLWLPDTTG

SEQ ID NO: 62 - protein, 19 aa, synthetic construct
MKHLWFFLLLVAAPRWVLS

SEQ ID NO: 63 - protein, 19 aa, synthetic construct
MEWSWVFLFFLSVTTGVHS

SEQ ID NO: 64 - protein, 6 aa, synthetic construct
HHHHHH